SEQUENCE LISTING (303 sequences)

SEQ ID NO: 1 - DNA, 401 bp, homo sapiens
tgttgctcttcccatagagttcccgacacaccaataacctcctacctctctacccatgggatttcggactgtttccagtactccccatcattgtagagtttatttctaaaaagcccacccacgctatagatgaaagctcaaacacagaggctgagaagtccagggacctacagctcataaatgacagggtcaggacctcagcctggtcagttgattcctggccaaagttgtcgttggcccttttttgagaagctaaatataactttgcttacaagttcaaaggaaagtgattcaaaacttgctccgtcaaatgtcttgattttaggattcacaggtagaaataaaatgagtgaaaaagcactgaattcatgtgagaatgggttccaaaaaatatagcagat

SEQ ID NO: 2 - DNA, 401 bp, homo sapiens
tgaatattggataggcagttagcatggtttgtcatgttccgcaatttcttttgctttgggagtcaaatttctgaacacagaagatatttctagcaatgttaatagtgtttcaggaacttatcatacttcattttttaaaatgaaaatctcactttgacagtggttttgagaattggatgaggcacataggtgaaggacctgtaagacttccctgacgtagtgactcaaatgatgtcaaacattcatcatcatgacttctgaaaaactgtaccactttccagcttttgtttccacttttcccctttaaaaaatttgtagaagtttaagaggtacaagtacagttttgttccatggctgtactgcatagtggtgaagtctggactgttagtgtaccccccact

SEQ ID NO: 3 - DNA, 401 bp, homo sapiens
gtgaaggacctggccctggctcagcaaggttattgcatttaatcttcacaacaggtattattatcattttataggtaaggcagctctatttttcttagggctagctctacaaataattttttttgtcttagaacaagctgatgtccaaaatttgctttttatggaaaatctgtcgttgtaaaaatatgactctggagacacattttctgagagcagtaaataccagaagcccagccaaaaaacttcagcaccctgttgagattgggaaaaggcaacattgacttattttcacttaagtttgtgacttagaaaaaaactaacagtgatggttttcttcttctctgttaggcttgtggctgcaaattctttgcatggccgtttatttcactgaagcgaatgat

SEQ ID NO: 4 - DNA, 401 bp, homo sapiens
  variation 31: /replace=“t”
tcttccagaaaactgcctgcaagcaatttccttaatcgggttgctaagggcaaccccaaagaaacaacataccaagctatctgtctgtccatccgaatcccaaaagctttccttgtgcttcctgctctgaccaaagaggcatttgaggcacatgtgagagaacagctttccagaaaccacacttgagtccacagcagagcggaaggggcaggaggcatcaggaccatttccagatttctctctggccctgattgtctagatttgcatccaatcactgcaagagtgtatacaaaagttgttagaaagatttgggtttcacacagagttatgaaaacaaggtagaagcctcatgtcttcatttcccatgaaatcttgtgggtgtaaatgaaacatgttttagt

SEQ ID NO: 5 - DNA, 401 bp, homo sapiens
cagaactgggaaaaagcaaacaataaatgtccattgtttaagccctccagtctgtggtattttgttacggcagcctgcgctaagacaccagccaattttgtttctgttttccctccctgaagtcagtgaggatgggagctctctaggtagcagtgtgagggtctgagggagaccaccgagggacatgggtggagagggacggtcatgggtctgagaggccagcaggggccagatcatgcctgctttgtaggacacagtgaggggctgatacttcaagatctgtgggaatgggagcgggagcccagggagggctgcagggaaggacttggctctggctgctgtggagggagagcaggtggtgggaaggcagggcaaaaagtgaggagtccacaagagacact

SEQ ID NO: 6 - DNA, 401 bp, homo sapiens
  variation 145: /replace=“t”
ttaggggagttttgtgaacttacttcatgtgccattgaatttctaataggtgatatctaattttaaattgtagtttagagaaaaaatgttgctattctattgctgaaacataattgatatataaggagagtttggctttgaatacatttctttctctgtatatatttattttagatttcatgagcgagtatttgattctttaaatattgaggacattgcaaaacatgattttactggatttttcagaatcgtaaagaagaataattcttttatagtttagtaactatctaaaataatttagacacttttgaccttattttcttgatgtttctttgagatgaggaggtagtaattgcaatttcactttttctattgaataaaaaactaaatgaagtgtatga

SEQ ID NO: 7 - DNA, 401 bp, homo sapiens
  variation 340: /replace=“c”
tttttcttgagcaaggtatgtataagtaagtaaataagggcataatgtcaggtaaactgaatggctggaaagtggcaaccaggatttagttatttggcattatcacaggtttttcagaaacattcaatttttagcaaaaaacaagtcccctccaaaattcaatctactgcaattgatttgcacgtggtttaatgctgttactagtctataaataatggactcaaatatttgaagttgtagatgctttcatactgttttaaacaaacataacttatatgcaaatggttaagaataccagaaacattgcaagaatgctctttttaaatcctgaagatattggtttattttaaagcatcaaggatgatttagatttttggaaaaacagaggtggcatgaagatt

SEQ ID NO: 8 - DNA, 401 bp, homo sapiens
taggcgctgatttttgaaaatgcaatctttatccagagtatttgacaaatgctcagaatgctcagaaatgtattaatatacagccagacagctgtcaaagcagcaggcttctcccctggagagttcattaacctcatttccacttgctgagtaaggagctctgtcttgcctgagagggaccacggtaccaaaatgtacgggtttcaagtgaggagtgagaaaaataactgaagtatgagaaaataaagatttcttgataaggcccaaagactagctcatttcctagtatgtgttttaaatgcgcgttttctgtttattttacttacctatgcatttatctattcatctcttttcaaaatgctttgaaggtgcaatgatgatatgctattttatgaactaat

SEQ ID NO: 9 - DNA, 401 bp, homo sapiens
  variation 275: /replace=“t”
tgaatgtgtgtgtgtatgtgtaagtgcacgtgcacatgaatgactacgtatgtttatgcacttgtgcatgcatatacatgtacgtgtgcttgtatgcatgggtgtgcattatgtgcgtgtttgcctgtgtgtgcatgcatgcgtgtgtgtgtgtgtgtgttctctgtggagggaggagactggagattctcttccccaacgttcgaggccagtttgccaaaaatcttggtgctaactgtatgtccaggttacagatccacacgcaaatgcagacccaacaacactgaggctcatcttttgggtttctccccatcttcttcaccatgactcagttctccaagaaaaatgttgttgctactttcaggaacagtccaggatgagagcttaaggtagagtccgga

SEQ ID NO: 10 - DNA, 401 bp, homo sapiens
  variation 266: /replace=“t”
gttcacaaggcaaataaataacagatgtttgagattatggatatgctaattacccttacctgatcactatgcattatatgtatcgaaacatcactatgtaccccatgaatatgtcaattaaaaaaatagtttcaccaagacccactaaatcaacagaattttgtggcaacattatgcagaccccactgttgagagtaaacgctatttgatatgatatcaagttccatcagtttgcatcagatcatcagatgagagggccataagcgtcttctgttctcataattatgcagatttattgttttgaaacaaaatcaacttctctctaaaataagcaaggacactggggcaaccaactattctcagagaaggagtgtttttaatttttttcaggattcttagga

SEQ ID NO: 11 - DNA, 401 bp, homo sapiens
  variation 50: /replace=“t”
taatacacacacactagaaacctaattggttttgaatcttgagcaattgcttagaaatgttcatatgatgaaataaaatgctttgtattgcaaacttttatttaagaatgagagcttcttaattccgggtcgttggacaatatccaaagtcaaatggtagagacatatgaaagatggttagtaattaatcacgcctcaccggtgtgccaggaaagtaaactcaccaggagccactctcagtgccttatcaaattactaaaacttgacttgctttcctggattagtaaagtgatacactgttgattctggaaattcgtaacttaagatgattcctctgcatagtattcagcaaaccataagatggtattttgataattttgagtttcatcaaaatacagatt

SEQ ID NO: 12 - DNA, 401 bp, homo sapiens
  variation 18: /replace=“t”
tgaatcttgagcaattgcttagaaatgttcatatgatgaaataaaatgctttgtattgcaaacttttatttaagaatgagagcttcttaattccgggtcgttggacaatatccaaagtcaaatggtagagacatatgaaagatggttagtaattaatcacgcctcaccagtgtgccaggaaagtaaactcaccaggagcctctctcagtgccttatcaaattactaaaacttgacttgctttcctggattagtaaagtgatacactgttgattctggaaattcgtaacttaagatgattcctctgcatagtattcagcaaaccataagatggtattttgataattttgagtttcatcaaaatacagattattatcctgcgtgagtccactgtaatcttagc

SEQ ID NO: 13 - DNA, 401 bp, homo sapiens
aggaatgagttcttgatgactccatgtgtgtcaacaagcagtttacgacttctcctttaaaacagaaaacgaacaagagtgtagttttgaaaagtaaatttatttatttatttatttttacttaggcttatataattttctgttctgtaagagagatcttttagggcccagggaattattccataccttggggacccggggaaagactgcgtacctggccaagactgatgatcaaccaagtgggatgctttatttcctctttagttagaagtttcaggatcaaagtgagcctttgagctatttgtcattttccctctaaatatttttgtcacacacacacaatttgtatagtggcttcttcagaagcctgtggcaatccttcctggctgtgaaaacagatg

SEQ ID NO: 14 - DNA, 401 bp, homo sapiens
  variation 348: /replace=“g”
aacaacttctggatatggataatgttaattcttgtccatgatacaattggagaaattgaggcttaaagagatgaagtcacttttcaagtgctacatagatgctaagtgataaagaagaaattcaaacccaggactcttgattgcaggcccccagctcttatcacagacactatacaaacaggaatactttgtgttaccctgtgttaatggttctcttattttgctgtgcatcagaataccctggaaggatgactaacatacagattgccaggctcagcacctgctgtgtctaagccaggatatctaggggcatttctagtaagttccaggtaatgctgatgctgctgatttagggacaacactttgagaaatatggctgtgtattatgggtctatttgaac

SEQ ID NO: 15 - DNA, 401 bp, homo sapiens
ccctgaggccctcagagtcatacacagaggaagagcttgggtcagactttggccttttctgcttgtttcatcaaaacagaaaccacaagctttatcacagcctcaaaatgcattttctctcttctttggggttaaaaacaaatttttcttcaaagaaagcatattgagatttttaagtcaagttcacaattaaaaaaaaattttcttgaaagatgtgtgtattgtcttaacatgggttccctttgttaacacctcccagtggatttatttggcatgagggatctaggggcaggaccctggacttcttcttagacaacctaacctctttggtccagaactatctcaaggttctgctaccaacatgttagaagcaaaaggcccaggaaacaacccagtcactt

SEQ ID NO: 16 - DNA, 401 bp, homo sapiens
  variation 378: /replace=“c”
gaattgaataaccatcataaggaaacaataaaacatgaagtgactgagaccagatgatcaccactgggcacacgtggtctctgtagcaggctcagggagcccagggttggctttcagtttcctgcaacacagagcagtcccttcagcactctgccaataaggatcccatcggttaatgagacgagcttcaactaactggcggaggctcggagggtggggcacccacgttacattttctccttgcccacaactctttgaattttagtctggttgtcaattgttctagcatctcttccccctagtgtgctgtttctcccctcataaaaacagctacagagctgaggtctgaaatatctggagtatataaagatggtgaggattggaggggttcatggtgctct

SEQ ID NO: 17 - DNA, 401 bp, homo sapiens
  variation 319: /replace=“g”
cttgagttttaaaagttgtcagaagaattctgagaagatgatagagtaggaaataccagaaatccctctccccatctagacaataatagcattggcaatatctgtctgacatgattattttgggattctggagtctattgaatatttgcaacttctaggggaaggcttggatggtaaattgtttaattttgatcattttctgctcctagcacagtagctgctacctatctttcacctctggaccctcctctcttgagcaggcagcatgcatgtgttcctggagaagtttgcacacagcttgttggaggcagggtgggcaaaaggatcctgccctccaaatatcagggatttgtactctgactgctgatggctgcttctgatcacaggggtgctggcaatga

SEQ ID NO: 18 - DNA, 401 bp, homo sapiens
  variation 265: /replace=“t”
acagattaaatccctatttgaaataaaagtagctgtttactcatgtaaatgcattgtttttccttgactacttaaaggcaaacagattaaaggacatggattgtctaaagaggctttggtgaagagtatgttgaaattcctttttttttccctactgcaatccttttcaccaattcatttaggctttctatggcttttaagagatgagagagaaaagtttttgtttctaattaacaaggtttcacaagcaggcagaggcattacagtttccatttaaaaagaaagaaaacctagaaaaatggtgacatgttcctccaccggcaggatgggggtaagggacaactttgggcatgccaagatactttcttcactgagagatttattttaaaatattactttat

SEQ ID NO: 19 - DNA, 401 bp, homo sapiens
  variation 137: /replace=“g”
tgactacttaaaggcaaacagattaaaggacatggattgtctaaagaggctttggtgaagagtatgttgaaattcctttttttttccctactgcaatccttttcaccaattcatttaggctttctatggcttttaaaagatgagagagaaaagtttttgtttctaattaacaaggtttcacaagcaggcagaggcattactgtttccatttaaaaagaaagaaaacctagaaaaatggtgacatgttcctccaccggcaggatgggggtaagggacaactttgggcatgccaagatactttcttcactgagagatttattttaaaatattactttataagcaaaagggcaatctatcattggattttccaacaccatgttggtgttttaattaaaatagca

SEQ ID NO: 20 - DNA, 401 bp, homo sapiens
  variation 378: /replace=“g”
ttgcctccaagttctggcaattatgagtaaagctcctgtaaacattaatgtgcaggtttttgtgtggacttaagttttcaactcctctgggtaaataccaggagcaccactgctagattgtatggtaagactatgtttacctttgtaagaaactgccaaactctcttgcaaagtgatggtattattttacatttctataagcaatagatgagggttcttgttcctcttcatcttgccagcatttggtgtagccagtgtcttggatttttttccacagaatgatatttttacatatccctgaacaagggatgatgtttaaaattcttagcaatgagtatcaccagggcaggagctcacagaacagatgtggatattcaagtgaagattggctgatcactggc

SEQ ID NO: 21 - DNA, 401 bp, homo sapiens
  variation 23: /replace=“t”
agtgctgcttgatgcctattacctttatgtaaagacacagccagctcacgtgagctcttaggaagggggatagagaaataaataccctgacctcatcttcctgccctctgggctgctggcctccaaaccagaagcagggcactggagcttaccaacatggtacacaaaggctggcatcctggagctcagagcagaggagaggaggtcagacagtggatctagaggggcagacaaaaatatccagaacaagtttcactctccatgggcctgaactagtggttacaattgctggaaggtagaaagtgttagggggtgttcaggtatgggatccagatgaagtgggagagaattttagtatgctttgtgaagacttcccatgatttgaaattgtgtaaggactg

SEQ ID NO: 22 - DNA, 401 bp, homo sapiens
ccagagttcccatgattctgcacagaggaaggttctcatagtaaggatctgatgctgtgtctgcaggcttggcttggccatgggagtatgctctgtccatatgcagaagtcactcctggagcagctctcaaccagtgactgagagataaataacctggcttcctcacccctcaggtgagctgtgttccacacagtctaccggattcctccagggggatcgagcctcagctgctcaattattacctcagtaataacttactaattaatgcacctggtgttcactcctcttctccatttcacctctccgtttacctgcgtgcttcctgggattgcatctcaaaatgaactaaggaagtgaaatccttaccttagagcggaattctgagggaaacccaaactaa

SEQ ID NO: 23 - DNA, 401 bp, homo sapiens
  variation 173: /replace=“t”
tcctttgtaagtcttttgctcattcatcaccaaggttagaagtcatttctctgtgaacccttccctatcagagcaggtctgagcatgtggtgtccgttcagtgggggtaatgtgtgggcagctgaagtctgattcgtaccttccagaccaaaagaaaggcactggttccagcgtagtgtcccatagcagaaacatggctcggagctcagtttaaaggtgttccttgaatgaggccaatcctttttgggggaggggaaggggaaggagggtctcccaacccccgccttttttatttgagaaagaatctcattctgtcacccaggctggagtgaagtggtgcgatctcaactcactgcaacctccacctcctgggttcaagagattctcctgcctcagcctcc

SEQ ID NO: 24 - DNA, 401 bp, homo sapiens
gtaatcactgtgatctgggagatgcgtttggactggaccggccagtgaggggcctacctacctgcctagatgaaaactatcacttgcacagaaatacatggaaagatgaagaggcataaagttaatggagattaattgtaccagctttgtaggagcacaggaataataaccatttatggaacaaatatttctatgtattttttttcaaactgtaattaagggtaccattgtccccaattctttacccctccctttatctctatcctttgccaagtgacttgcagtcccccacctctatgggaggagtatatgtcctttatccttgactctgggcgtgaccttgtgatttgctttggccaatgacaccaaggggacagtgacagtgttccagttctgatcct

SEQ ID NO: 25 - DNA, 401 bp, homo sapiens
  variation 74: /replace=“t”
ttttatgtatcaacaatttaagttctactgctttaagattatatggacattaaatgtacagaaaaatagaaaccaacaaaagataaataaattaaatacatttttaaaaaagtctcgcgagttgtggtggcttaatattgccgtgagctaatgccttgagggctactctctctggagtgggaatggagctctcagtaattcgacagttttggccagtggcttgtcacacggattgtgcattcacctggctttaggaggaagaggaggcatggcagccctgccttcatcttcctctttactttgccctcactatcactgttaacttcgattcatggatgcttttcagggatttttttttctaagccacctagccaattcgaggggtttagtgtacttaaaac

SEQ ID NO: 26 - DNA, 401 bp, homo sapiens
  variation 237: /replace=“c”
ggatttttttttctaagccacctagccaattcgaggggtttagtgtacttaaaaccagatagtataatccataaacccactaaattgggcacatgtgagctgcaatatgtcacaagatgcagaaaaccagccctgagtgagggcactgccttccgcagctgtcctctggatctttcctgtaatgtcagtgacacatgaccgctggcatgcagcctgctaagggacggtgtccatctgcatatcaaccaagacaaaagcctggctcatcatttatttagagaaagcaaacataaatggaagaaattatattttcctcctattccatagtttatcatgtcttgccccacctgggtagtgtcacttgtcctgaagatggtaggagtgagggaaaagaggtgagc

SEQ ID NO: 27 - DNA, 401 bp, homo sapiens
  variation 89: /replace=“g”
aatggatgtatggagatgtttctctccagcaaagataaggaaatggctggaagactgcgaaagaatagatgtgccactttggaagttgacataaaaatgaatgatatttttactttttggaaagccaagcaggtcccagagtcgtggggcagtacttggcctcatctctgaggccacccattctgctgctggcagccacagtgatgtacccttggcactatgtcgctccccctacttggcatcctcagtttttctaaactcatgggtgagagacgaatgacgtcagaggcccaaacctttggatgctgatctaaaagcatcaaccaaaacccaacacccgagcttcgtaagttcccatttgaagcaggagcagtttttatagcaacagacaaaggattgct

SEQ ID NO: 28 - DNA, 401 bp, homo sapiens
  variation 24: /replace=“c” /replace=“g” /replace=“t”
ctaagacagtcttttacaatctaacaaaaacctttctgctgtggaagttctgtggaaagggtcacagatcatgtgcagtcacacaacactctttggattatcactgtcatgaaggagcaatgggggagaccaactcctccacgcagggctcaagatgtgtgcatggtaggccaagagggcaagtgggtggggaggtggaaggggtcaggaaggaggcacagacaccgtcaaagcttaaggagaacggaccgttgacacatggaaatggatactgtgacacttagatttatcgtcaggcaaactgttcaccacaaacccaaagttcctctaaacaatgcctaggaaaatcttccagaagttttgaggatctggtgaccatattttcagaatcaaaagttggg

SEQ ID NO: 29 - DNA, 401 bp, homo sapiens
  variation 137: /replace=“c”
taggtaaatatttacaaactgaaggtcaaacacaaatgtctactttacaactactatactgcattcataatttctctcctcattttatactctgatgaaataccaacttggtcatttctgtgcattgtcaagagggtcgaagtcgaagcagaagcaagcagctggctggcttagcttatggttcctctggcacaggaaagtggtgagagtcattattcttattatccaggagttaacggatttttgcatgactgggctgagtgtatgtttattttctggcccttgataattactgtattcccattttcaatatgaatttgtttctatccaatggatggtcaacagtagccactgtaattctttttgaggggctatatatgcaccattatttttcaaacttg

SEQ ID NO: 30 - DNA, 401 bp, homo sapiens
  variation 93: /replace=“t”
ttacataaagtcaggctgacatgggttcaaattctggctcttcacaataaccctataaggcaaatactattcccgttttacagctgaaaaacctgaagtcaggaggtcaggctaagttaatatgtctatggtcttatagctagtgggtgggagatgagaatgaatccagtctggctgaagagtccatacttctggcctccgtcatagcctgggcttcccaaagcttgtgcttttgttccttattggcaacaggccatcaactgcattcgttacgtaaggttattatgaggagtaagggagttattaaaagtgtctagcatggttcctaggacatagtaggtactcaaatatttttccctttctgttactattaacactttctttataaaataaaaattggc

SEQ ID NO: 31 - DNA, 401 bp, homo sapiens
ctaacactgagaaactccagacagatcctgaagttaacaaaacatgaatctattatctacttaaaatgactgcaataaaggcttcttgatctataactctagttttgcatgaagatcaacaagctttcacatggtaggtggagtaaatgtgaaggtgaacagtgtcagatgtgaaagatacagtgggattcttgccatacgctcttcagtcttaaaccttctgcaggtgatccaactacaccccaggaattaagaagcagattataaacctcaggcctttaaagggcaaaaaggtctgaattagtccaaaagttcagggcaaaactctaaatgacttttagttacaacggaaacccacttggaaagttgcttttcttctccaatagcttcccaagaaaggg

SEQ ID NO: 32 - DNA, 401 bp, homo sapiens
  variation 19: /replace=“t”
taataataatagtgaaggcctggactgggtagaggaagaaactgatccaaaacaaacgtgcaatgaggcctcagccaattcctggggggcagatctggctgtgtacagcagttcctgaataatgtcggttcattcgatgttctttccttataacgttgatgagaaaacaattgatttgcacctggggccaccatctgtgtgtatttgcacaacctccccataactgcatgggttttctctgagtactctagtttgctctctcattccaaagtcatgcacattaggttaattggcatgtctacatggactcagtgtgagtgaatgtgagcatgggtgtgaatgtgctctgccatgagagactgtcctgttcagggctggttcccacctcactccgtgagctg

SEQ ID NO: 33 - DNA, 401 bp, homo sapiens
  variation 8: /replace=“t”
ttctttccttataacgttgatgagaaaacaattgatttgcacctggggccaccatctgtgtatatttgcacaacctccccataactgcatgggttttctctgagtactctagtttgctctctcattccaaagtcatgcacattaggttaattggcatgtctacatggactcagtgtgagtgaatgtgagcatgggtgtgagtgtgctctgccatgagagactgtcctgttcagggctggttcccacctcactccgtgagctgctgggatgggctctggccacccatgaccatgaactggaaataagcaggtaaagaattatcttacttggttttattaatctttcttaaatgtatgtatagttcacatttatttcaatgtttaatattagaaatgttttgc

SEQ ID NO: 34 - DNA, 401 bp, homo sapiens
  variation 45: /replace=“t”
gtgtcattagaggtacagaaacacactttttttttctatctgtggtgtctcaaattgttatggtggtgttattttcttctaaatgtctttttaaggaaagaaaaataaaaatttaaattattagcaacgatttttgccattcatctttatattatgcattgctaattttaaacacaagtgtgtttaattaacttgtatggcgactcaccaaaatatacattttgtattttattactcatgcgtgtataggtttttgttctaacaagaagagtaaaaacactgcacaaaactaactcaaatgattttatttcacttattgatatgtgcatattctgccaatgttctctacctttggcatactggtgactaacagagaactgaaaggaaatgtaactatctga

SEQ ID NO: 35 - DNA, 401 bp, homo sapiens
  variation 50: /replace=“t”
aaaaaagtttcaaaggcagtttttaaaatgcttagttaaaggctaacatcagtatagtttaaaaggtaattgatctatagatgaactaacaactaccaacagcatctcttttgtcagtctatagctgacagatccctccttcttatgcctacaagtctgacatctactgaagtttcatagattggctgaaagcagtagtccggaaaactttagtcccagttccagagattttcattctctataccatcagctctttggaaactggtgccttttaggctctgaattgacatgggttcaagggcatctcactctgttgaccttgtaccttgaatttcctcaacattttctttgtgatttgatatcaatattagttctctagctacctgcattacaatagtcct

SEQ ID NO: 36 - DNA, 401 bp, homo sapiens
atatagggtcctccatagtatttgcaaataattttaaaatttccatatttgtaatttgccattttaatttttctaatggtgttaacttatatcttctctttttattttaaaatttgtattgatacataatatttatacatatttatggtattcatgtgatattttgttacatgcttaaaatgtgtaatgatcaagtcatggtttttagggtatccattacctcatgtatttatcatttttatgtattgtgaacatttcaagtcctcttttctggtattttgaagtatgaatatagtgttgttaactatagttaccctactctgatatcaaacattagaacttatttcttctatctaactgtatgttcgtgcccattaaccaacctcccaccacacaccctt

SEQ ID NO: 37 - DNA, 401 bp, homo sapiens
  variation 28: /replace=“t”
cctggatgtctaagtctcttgctggacctgagaagttttcagctattattttgttaagtagattttctatgcctttgaccttctcttcattttctgtaacattcaaaaatcaaatattggtcactttatggtatcccatatgctatctaggcattattcgttcttttttatttagtttttaattcatttttgtctgatttgatgatttcaaaagacctgtcttcaagttgtatgattttttcttctgttcgatcttatctcttgttgaagactttgtatttttaatttgattcaagaaattccttagtttcaggatttttctctggttcttttttataatacctatctctttggtaaattttcattaatatcttgtgttgctttttctgacttctttgtat

SEQ ID NO: 38 - DNA, 401 bp, homo sapiens
  variation 160: /replace=“t”
atggtgtgctgtagctgcttaaagcccacagcttgtgtgggactcagcaggaacttctgttctgatgtaatgctatcgtgtgatgcccagacatcttcttctgttactctcagggcttgagagggtagatgggctctctcatggctaggattgctagagctgcagtggaaatgtggaccactggggctctctcacttatactttctccacttggggagctcctctgggatcccagcttatcccagctgagcaggctacttcactttgctcctctaccttgccttaggtgttccctgtcacttctctgttgaattatagtgttctttcttagatgatctattcaaagtgtggttatctactcactatttttgtttttctctgtgcaggaggtaccagatgcc

SEQ ID NO: 39 - DNA, 401 bp, homo sapiens
  variation 274: /replace=“c”
agcaggctacttcactttgctcctctaccttgccttaggtgttccctgtcacttctctgttgaattatagtgttctttcttagatgatctattcaaagtgtggttatctactcactatttttgtttttctctgtgcaggaggtaccagatgcctctagtcagccgtcttatagcccctcctatatcgttttaatttttgtcttggtcaaacttgcaaaaggttgtctactttgttgatgttaaaattttttcgttttatcgaattttgattttgtaattcacacttcaggcttttatatttatccattctgtccttgtagcttagttggatttattttgttgttacttcctgtgcttctttgtttgacgtattagtttacttatttacatccccaccagca

SEQ ID NO: 40 - DNA, 401 bp, homo sapiens
  variation 247: /replace=“g”
ttgagttgttgggtaccagaactgataggtgtcatgtatttaagcccatttgaaaaatacatttcaataatggagtgagactctgtctcaaaaaaatttcaaatgtcttttctcaattgaaaaaacctattatttctagatagataagtaaataacctttgcaaactcgagccacacttttcccctttcttttaaaattctgcttaatataattgaaaactgaagtctgactctccaagcctataaaggataaaagagagacaggatagatagaaataactggcaacgaagagttactgggctttggctccatgggagacaatagtggtttcatagaaagcaatggtggactcttagtcaactgaagcttgggtggaacttagctgcataattaagaagat

SEQ ID NO: 41 - DNA, 401 bp, homo sapiens
  variation 121: /replace=“g”
gtttctggcctgtgcttctacaaaacacacagcacatattttctaaccataattgaaaccattttaaaatagtgtttttggaggagtgaatctatgtgaaacttctgtaagctatttgtaagacagccgtgaaaatcctccatgttctatgcattggattttttttcctccttactcacaaggatcatcttcaggttaatgtcacagaaattgggacaacttagtgttcgagaaatagagaaatagctcaaggagctattagataccaacccataccatgttgttttctgagggtgggagaaactagaagaaagtttgacattactctcattccatgagacaacctcaagaacacaaacccaaactatccaggtaggacttttagtttagggagaccacca

SEQ ID NO: 42 - DNA, 401 bp, homo sapiens
ttaagatgacgtatcctgtaggatggactaaataatgctgcagcaaggaatacttcccaaattgtcatggcttgcgataagtcttatgtttttctcatgttttatgtccattatggatcttgccctggtacccagcctgatgcagcagctgctctctggatcatgcctcatcgctgtggcagagcttattgcaaatgagtgactctacctgggagtgacacataactccactcacaattcattgtccaaaataagtcaatgaccacacctaacttcagtgggacaaagaaacactatcctactctgtgcttagaaggaagaaagatgaaaatatttggtgatggctctatgactaccatggatagcttttattttaatattgtacattaatagcgctctct

SEQ ID NO: 43 - DNA, 401 bp, homo sapiens
  variation 103: /replace=“g”
gagtatgcaagtacatgcatgtgtgtacttcgcacactgttatctattttaggctcaaatagatgtatctatggttgagaaattcccattaaatattggggcaaggttcagatgctgtagatatttgtttacattttatcaaatgctttcttacatgtgaacccctcaatctctgagaaatcacctaagggaagccatgggtctgcctcctccaggtttcattattgcatggaatgtgaggggatttctgtcttaattaatgcataagccagctcttctgagtttgggtggatatccttgaggagaaaggtagctaaaattagcagcctctactctcttctgtgttccttataaaaaaaaagagaaactgtctcatctttaagaatggaccagttatgaac

SEQ ID NO: 44 - DNA, 401 bp, homo sapiens
  variation 90: /replace=“g”
ttgttctcaccaaaactcatgttgaagtttgatttctcattgtggcagtgttgaaaggtggaagctgatgggaggtgtttgggtcatgaaggctctgtcctcattgatggattggtgccattctcacagtagtgacttctccctctggcaagactaggtaagttcctacaggaatagattagtttctgaaagagtgtgtcgttataaaggcagcacgctcctcagcttttctctcttagcacgcgtccatttcccttttgaccttctctgccatattagaggcaacatggaaaccctcaccagaagtcagggctttgcccttgaactttccaccctgcagaaccatgagctaaataaacccattttctttataaatcactcagttctggtattctgttata

SEQ ID NO: 45 - DNA, 401 bp, homo sapiens
  variation 45: /replace=“c”
tatcatgagtatatctaaccctatgtttactgtatttgtgctatgatatgaatatattttgatttagtatttgatcagtctttactgattgttgagaatctactaactcagtgcttctcaaagtggtaaagggctagtttttcaaaatttcttatctctcctggattggtgcatagtcttaccacatatgactcatctccggcttgcaccacttgctaatcaccacgtaagtgtggcagcatctaaacaaatctgtcttcatttcaaggaagagaacgctgataatgcacatggatgttgcaacaatgtcaaattgctatgaaaatttctaaatgaattttctgctcaattgcctaccttgttgcaaattaataacaggcagttaacataactggtagtgt

SEQ ID NO: 46 - DNA, 401 bp, homo sapiens
  variation 314: /replace=“t”
acagggtcaggtgaggaatgtgaatacttttaggatggggcctttcctcctagatgtttctaggtggtggtttccttatgaaatgacgacaccccagagtcatgaccaaaggctcatcacagtcagcttgaaggatgccctgtgtcagctcctacttctgccccactgttgggctggcacctacttcttctgaaagacacgcctcgggtgtcccctctgcggtggcacaaagtggctctcagtgaaccttacaataataaattataaattaataagtaattgatagattgaataaatgaatgtttggagagttcgtctttggttagatacattttcaatatgggtttatctgttgatcattcagaaataaaagcgatatttaactgtgtctcttatttgga

SEQ ID NO: 47 - DNA, 401 bp, homo sapiens
  variation 296: /replace=“g”
aggggaaaatgattaagtactgggttatttgagaaaccatgaagaccagaaggaagtgacacaagttttcaaatgctgaaagaaaagtcttatcaaaccaggattttatattcaacaaaaaattccttcaggaatgaaggattaacaaagatatagtcaaatgagggaaaactaagagagtttattgccatcagacctgtgctaaaagaattgcttaaaaaaattcttcagacagaagggaaatgataagaaatggaacttggaacttcaaaaataaaagaagatcaacagaaatagccacataaatagagtagactatttttctccttttgagttcttttttccttttttctatttgtgtaaatttagggagtacaagcacagttttgttacatggatac

SEQ ID NO: 48 - DNA, 401 bp, homo sapiens
tagagtgtcccaatttgaatgtcagatctggttacagatacattgagataatggtccctgaaccctgggcaactgggcatgttctggggttgctggagtccttgttctggtcacttccagccatataaacatacttttttctttgctgaggaactgatctgtgctgaggaatgccacatctgggctctgttgaagaaggtcggcaaacagaagagagagatggggcatttcttcctccactccctccctgccttggggtcacaattccagcagcagttgtacccttcccttctggtgggcacttcccctgcagccctcttccttagctcctagtatctcctagggcagtaatagctccaaactgctccaaactgctgttagtctttgggggcctcccaacc

SEQ ID NO: 49 - DNA, 401 bp, homo sapiens
  variation 124: /replace=“g”
ttcatctgttcaagaaacaacaatgaaaaaaaatggtgattaaatgcaaccacacctaaataaggtagccaggaggtagaaccagcttctccatgaacaggccacataccatctcccggggctacctccagtgtccagcctggctgtgctcaagccctcagggcttgctggactctcctggcagtggggggtcaggactcgagtaggttggatgctgatagcagagagtgtgattctccggcctgatgacccagaatcactcccaggactggaatcccagctctcccaggaggaaggagtcgtccctggtctcccgcatcccccaaatgctgattctgtgtctgcatgtggaggggtaacaaacaaacaacaaaggaccatctcaattaacctgccacaca

SEQ ID NO: 50 - DNA, 401 bp, homo sapiens
  variation 91: /replace=“c”
ggatatcagaaaagtgttagaaagaggaaagtagagtaaatccaaacctctctctctacactctccaaacctgtcccccacacccaccccacccccttgttgccaagttgtaagatcttgggctgacttccaagttcccaaaattcaagtgtggaaaaagccaactaatctcctggaaaaatctcctgatgtgtatttttctttaacaatctgggctgattttaaacttagcccacagtaaactgtgctcaaccggcattatcattatggacacttgctgctgtattttccaaggatctcccctcccccgctggtattgctaccagatacagaatggaatttgtcaggacaagccatgtattactactaaaccattaattcagttagtgcattaggagtgg

SEQ ID NO: 51 - DNA, 401 bp, homo sapiens
ctgcagacatgtatgcactagcttccctttggacactctttttttaaaaaaatgaggtgacatggacataatttaaaagtaaccattttaatgtgaacaatttagtggcattttgtacattcataatgttgtgcaacaaccacccttactaattttaaaacattttcctcaccctaaaaggaaaacctgtacccattaagcaattgctccctatttctcccacctctggcaccaccaattttatgttctgtatcctggatttacctattctggccacttcacataaatataactacacaatacataacctgttgtgtctggctttttttacttagcataatattttcaagactcatccacattgtatcaggcatcagtactttgactggataatattccat

SEQ ID NO: 52 - DNA, 401 bp, homo sapiens
  variation 346: /replace=“t”
agaatatgtattgggtggagtattctgtgtgttaggtctagttgttcaagtcttctattttcttattcgttttctgtttatattttatccatgatttaaagtggggtattgaagtctctattactgcagaactatctatttctccttcaattttgtcagtgtttgcttcatatacttgaggtgttctgtgaaagggctgtgtatacaacatgatctccaaatgcagattgagccaagaaatgaaaaaatgagtcagacaaatccagtttgtcagtaaatggtgatttattagaggacttacagacagaaatgtggtcttgggtggctgcaagccaggaagaattcggcagtgcaaccctctaggcccagggcttatatatcttgggtggggggtaagtaca

SEQ ID NO: 53 - DNA, 401 bp, homo sapiens
agaaaacccaatgcaaggcaaaagagcttggttgagtcaccccaagtgacaaagtcatacttaagcttctggtttcagtcagtcatctttgctgagaggttggagaacacatactagttaagacaaataaatagcaatttaaactttggacatgctttcttctcttagctatgggagggacacatgtttggagaaaaatcgaagcatgtcaggagggatgtaaaaacatttttatttctaatcaacaagtttataatgctttaaatgcttttggtttgtaacaagttaaaaatgaatgtctatttgttttttttaaaatctgtcttctttgcagtatgatttccttggtgttagaagaaaaaaaactgtgccttatgaaaatatagattaaaaatgtctaa

SEQ ID NO: 54 - DNA, 401 bp, homo sapiens
  variation 176: /replace=“c”
cagcattcttaatcaaaaaattcaaaattgaaaatgccccagtgaagcattcctttgagcatcatgtcagtgttcaaaacactttggattttaaagcattttagatttcagattttcagattaggtatactcaacctgtattatctttgatgcaatgttatcattatactttatgacttttatcatgctttctcttagttctttgaacataattataatggctattttgaagtctttttctcttaaatctgatgttttgtcattctcacagacaaatttatgttttctgctttttctggtatatggctcatatttttctgtttcattgcatgcctcatcactttcttttggaagttggacaatttagatcatatgtattgcagcaactgtgggtagtagcc

SEQ ID NO: 55 - DNA, 401 bp, homo sapiens
  variation 258: /replace=“t”
attgcatccttgtttttttcctataataatgtgtctcaagttcattttgtacaattcctttccagtcttgcattcattttccaaaggagaacttgttcctttcaatgggaaatgatatttaaagatcacagcttgggtgacattatttctagttcttttcactataaagtaaaatgcttttttcactttacattttcaccgctaggaaatgattttttaaaaaagataaaatacatcatgaggtcatactggaaaaccttattcccacaaacgccaacataacaacttgttttatcctacaaaacacacacagcaccattgttactacatataataagataatagaaaaacctatatacaaatgaaaaagagccttaaacgttgttttacagttcttcttg

SEQ ID NO: 56 - DNA, 401 bp, homo sapiens
  variation 23: /replace=“g”
gccttgaggtctgtatagagacaaatagcctgagagaggaagaacctatttgtgtaagcatcaggggtaatgatgtggagatgtctgtgtgtctgtgtttgtgtgtgggtgtgtgtaacaagatagcgagagagtagacagagagagacagagagacaaagagagactgagagagagaggcagggagaaacagaatgtgtgtgtgtaagagagagagtcagagagaggagagagagggagagagacagaggcagagagagacaaagtgtgcatgtgtgtgtgtgtgtgagagagagagagagagagaaagagggagagagacagagacaaagaccaagataaagacagagacagagagagaatggaaagagagagcgagagagaagatgatgaactggagg

SEQ ID NO: 57 - DNA, 401 bp, homo sapiens
  variation 68: /replace=“g”
gtgaactgaagttgcagagaaatacatgccaccttatgatgaggaatacaacaaaatctaacaattgaaagtatccaaggatggtttaggatgatctggagagggaagttggaggataggtatgtgtgaccaaattatatgaccttaatgtcccatatatcttgagattctatgatttaaagatgcctttagtatttaatccatggcttgacccaatccatagtcaactttttaatctataacattttaaaagataagcaaataaagccaagttggttagattctcccacccaaatccaagcctaaacttatgtaattgacattggatttattagtcacaaaaattatgtaacacatgggaaatatatatttgtaaaaaaaaatgagagttttttaaggtc

SEQ ID NO: 58 - DNA, 401 bp, homo sapiens
  variation 395: /replace=“t”
cattatgaaatgaaaagaatccacaatggaatgaaactggtaaagtattcaagctgcgtaagtcctggttttatcaggtcacaaaaattaattctcatatcattctgttgttttgcaaacaaactgcaagattgcagagggttttatttattttagacagcctgaaaacagcaaggtgtacaataacaggaataaccaggtcatttgaacaggatcgtgacagctattgatgctacctgtccagcctggacatgatttggcctgaccaagcacccaagaaggctcagtgggttgtgagcacaccctgagccaaaagcgttatgggctgctccaaaccaagtgtacttttaacgtggattaataaaaacctgatattcgggttcagaaatatgacagttgca

SEQ ID NO: 59 - DNA, 401 bp, homo sapiens
  variation 7: /replace=“t”
accaggacatttgaacaggatcgtgacagctattgatgctacctgtccagcctggacatgatttggcctgaccaagcacccaagaaggctcagtgggttgtgagcacaccctgagccaaaagcgttatgggctgctccaaaccaagtgtacttttaacgtggattaataaaaacctgatattcgggttcagaaatatgactgttgcattgtaaggctacatctagagtagtgcctttcttctgggctccacatttagaaaggacagggacaaagtaaagaatttcagatgaagaggaacaaaatgggaaaggccatgaaattgatttgttatacatatttgttcagggaaagaaaggacagaggcagaatgataagaggagcaaggaacacaggacagagg

SEQ ID NO: 60 - DNA, 401 bp, homo sapiens
aagggataaaaaaagatatcctaggcaaataaaagaagccaaaaaacagcaggaatagctatagttatatcaggtaaaataaacttcaaggtaaaaaccatgaaacaagacaaataaggtcactatacaatgaaaaatgttcaattcatcaagaggatataacaattgtaaatgggtccaacatcaaagcaactgaatatgtaaagaaaatagtaatagatatgaagggagagatagactataatacaataatagtaggggacttcaaaactcatcaatggacagatcatccagacagaaaaccaataaggagacattgaacttaaactataatttagaccaaatggacttgagtgacatatacataacattctacctatcagcagcagcagcagcagatt

SEQ ID NO: 61 - DNA, 401 bp, homo sapiens
  variation 117: /replace=“t”
ctttgcccatgcttccttagtaggaagaaaaagagaaggaaaataatggctagtaagtggataatagcaagttagaggatcctttgcaatcctggctggctctgtcctgtgcgcctcctttccttatccttaaaggtttgtgaacttcttgtcaccagcagacaattgagattcaaacatatgtcagtgtcattctgaacgaaaaatggaagtgtgatccttaagaaaaatggctctgcatattatcatagcttcttagatttggaaaataaccatggtgtctggaaaaaattgagagaatcttactttcaggttaaatgccatatccctttcaactccaaatacattagtatttctaaagtaagaataaatgattctgagactccgttagtgaaaactaa

SEQ ID NO: 62 - DNA, 401 bp, homo sapiens
  variation 98: /replace=“t”
ctgccaggaaagaaaacgtgaactgacatgtaactagccctgtctggcacccaccactttacttattaggtggcatctggtccctatacccacgtttcggtgtcagtttcttcatctttatgaatgatctgaggctgggagagggttggccccaggtctcactcattgaagagatttgaatccaggcgcatctcattccagcacccatatttatccagtgcattctctagggcaagatatttcccttacatgttttcctcttaactgcttaaaaagttcttttggcacagtattcctgtattctagtaacaaactacaatgttatgtgtcagtattcctattccataaataagaacctggaggatgtgaaataagtgatgtgtaaatgcgtgaaagatagc

SEQ ID NO: 63 - DNA, 401 bp, homo sapiens
tcttactccccatccctccttgaacctgggcttgaccaatagaagacaatgggtttggtgctttgcaacctctgctcctggtcctgagaggtcttgcagcctccttctgggtgctggcaactctttcttggaaagttcaccctggtgcaagccggcaactatgaacaaaggtaaacaattctcagaccaccacgctgtgagaagcccaaatgtagggaagggtatcagacatgagagtgaagaaattaccctggaagttggtcctctagccccagccaccctggtggatcagaaagaaaccgcccaacccagaaaaccataagaaaaataaatggttatcagaagtcactaacttctgaggtggtttgttacagggcaagaaattactgggacatatattc

SEQ ID NO: 64 - DNA, 401 bp, homo sapiens
  variation 40: /replace=“t”
ttaaagtaatccacacatgaatatagagaacaaaaagaacgtaaaaatgaaaatgtattttttaatttgagaaacatgttttatcaatacatatacacaaagatggcaattaaaaaggaaaacagacttactgtgaataaaacacaaattttgtctacatacactagaaaattttcctcagatccttggaacccgttgatgtatttttatagaatagccgtgatctaatttttggcaggggtgtgttcacatgtgtgcgtatccattcttagaaatgtgtaaaatagtataccaaagttaaagtaaatagaatgaaacaatttttctctacttagtaacatttttcagtaactctcctgaaacaatttccatctgcaactttacctttaaaagctttaatt

SEQ ID NO: 65 - DNA, 401 bp, homo sapiens
  variation 18: /replace=“t”
tttactcttgatcaaatcagcattttggcaatgaaagtcagaagacgtgtgttttacaaagtcaaaatattgttttattttattataatttgaaattacattttaaccagtctaatggctatttctgagccttaactgacatgaaactgtctgtattggtgcaattataatgcaagatatttgtagcttcatatgctgctgcagaggctaaacgcagaggatttagcctcctttgacactcaatcattgtacatttatcttggaaagggaaaaatgtaagtggctgataactaattttaattgacatctagaaaaaataaataaaccaagcaaaccaaatgtgttttcttaaaactatgttcttcttagctattagcctctctttagctacagaaagaaat

SEQ ID NO: 66 - DNA, 401 bp, homo sapiens
  variation 5: /replace=“g”
ataaagagtagagatcttctgctaactgtaaatggaaatatggattagagagaaacagacatgcactgttttaagccactaaacagtagccactacccattggttaatacagataagaaaactctcacaaaataaaagtgagggggaaaatagggtaaaaaatgtgtcctgaatgtatgaaatcaattttgtgtgtgaggggaaaataatgtaacaaatatacacacaaggaaaccccctcccccaaacccaccagaatattgtttttacctctgggtgttgagactatagagttttaatttctttatatattcttctgtttttctagaataaatgcatattattttataattacaaaggctactttttaactcttcataatcataacttataatggtaaa

SEQ ID NO: 67 - DNA, 401 bp, homo sapiens
  variation 249: /replace=“t”
taggcaatgagaagaaaatcgtaattaaggctgagagaatggcattgcatcgttgcagaggtaagaaacagcactgggactttaggaagtattttcactaggatcaagtataatttgcacaagggaaggggaaacaggagtagaaaagtaggcaggagccagagaataatatgccttgcatgccttactaagaatatggactttactccattctttggggagttatggacagataactgatatttccagccacaatcaatccagaaacacccctgttttgacaataccccagcctcctctagggggagccacctttgattttcatggcagggtgggatccagaaagctgaaagtctctttccaaacttgatttggagacatgaatttcacacaaatacaac

SEQ ID NO: 68 - DNA, 401 bp, homo sapiens
tgctgcaattgtatgcacatgaccatgaggcatgatttcctgaactgggtgggagataggggcacagaatttttgactcagttccattgctttaacctggaaaaaaaattccaacatccttggaactctgattttctgcttgtaatacatggagtgtaacagctgagccacaggctgttttgagaacagaacaaaaagctgtaaatatgtcagtcaataaaactctaccaagcaaagagcctttataaattcaaaacactattgatggttttaaattattttctctgaaaaaaacataacatcagaggttatttaacttttctattatttataatacagtcaccaatttagagataggagacttaattttctcttttgtccaaaatcaactcaaattatgc

SEQ ID NO: 69 - DNA, 401 bp, homo sapiens
  variation 152: /replace=“t”
acatcctctgcaacatctgttgtttcctgactttttaatgattgccattctaactggtatggattttggtatctcattgtgcttttgatttgcatttctctaatgaccagtgatgatgagctttttttcatatgtttgttggctgcataaacgtcttcttttgaaaagtgtctgttcatatccttcacccactttttgacgagggtgtttgtttttctcttgaaaatttgtttaagttctttgtagattctggatattagccctttgtcagatgaagagattgcaaacattttctcccattctgtaggttgcctgttcactctgatgatcttgaaaatggtcatattgcccaaagtaatttatagattcagtgctatccccatcaagctaccattgacttt

SEQ ID NO: 70 - DNA, 401 bp, homo sapiens
  variation 63: /replace=“c”
aacctttcagtggcttcttccttttggttattacgaataatgctgctatgaacatgggagtgaaaatgtctgttcaagttcccgtttttaattatttcaggtatatgccaagaagtggagatgctggatcatctggtaatttaacatttaatttttgaagacccactatactgttttccgcagcgaccgtaccatattacgtttcctaccagtaatatgagaaggttctactttctccacatcctcaccactatttgtctttttatgcttttgttttaatactagacatcctaatgggtttgaggtggtatcttgtgattttaatttgcatttccctaatgattagtgacactgagcatcttttcatgtgcttattggccatttgtatatctgctttgggg

SEQ ID NO: 71 - DNA, 401 bp, homo sapiens
tgaaataatccaactgcaggaaccaactcaatagggggaggattgcctaattttcaagcatttctgattcattatgtcaccattagcatagtattcccaaaacgcacttcgtgacatgcaaagatcccaagataaaaatttcacctatgtgtacgtctgcaaaagagaaaaacaattgtcagaatgagcactaaaccttcgttgatggttataggagggagtggaattataggacagacttaattttatgctttctcttctccagtgatttacttttgtacagaaaataagccatatttataattagaaaaagaggaaaacgtaactgattcaaaactacatgtacaatatgaattttagcttacaaatttatacatagaggtaaacatcaaaaacggtca

SEQ ID NO: 72 - DNA, 401 bp, homo sapiens
  variation 83: /replace=“t”
agttggagaatctactgactaaaagttgaaatggaaaattttaagactacaaataaagatcagactaaaggcaaggcagagccgccctgggagtacagtgaatggggcatctgtgtcccactcgggcgccgatcgccttgtccagctactgaacagttactttgagtgcatcaggagggagaaaattacaggcacagctggcatgaatccactccccatccatggccagtgcaaaggagacaagacactgtctcttcacttttatacacttacttcattttgtgaaacttctcttgcccttactatatgtgcatattgttcaagtgaatacctgccagttttagaagagaaagggttagtctgttcttggctgctacatcaaaataccataacctgggggc

SEQ ID NO: 73 - DNA, 401 bp, homo sapiens
  variation 14: /replace=“t”
ataataacaccagcaaatgtttagctaaaaaatttagccatacatgccacacattgtgctacacattgagctctttggtcctcacatcaacgctgaattaggagctatgattatccccattctttagattagtaagctgggtatcaccaaggagaagtacttgccccaaagtcacatggctcatatgtgttggaagcaaggtttgtgacagatcttccatgttaaaccattatgttatcctccttaggagagagtatggttttatgaatatactgctccaattggctgagatgacaatactagtttttaatcggcggctgtgacatattgagggcttatattaagaatgcgagctgctccaaaatgtccctgtcctgcagttgtacaatcacatattttta

SEQ ID NO: 74 - DNA, 401 bp, homo sapiens
  variation 24: /replace=“t”
caaaaactttgaagttagataagctgattcaaatcctaaagccagcccttgagtgctgtgtgaccttgggtaagttatagaacctttctgggcctcggtctctttaccttttaaatgaggccaagtgtgtacatgatgctaattaagtgagacagtccatgtgaaggcttagcacagtctctggcattcaataagaacacggtgagtgagttgtttttcttcctgtcattttaggggttgtagcaatagtgtttttgattgcagtgggttgttgactgccaagtgaatgattatacatggacatggaagttgagatatgaaaaaagccacgtgggtcagagtctcatggtcagctaccatggcaatcctgaaaagctcctctacctgaatggcctgtcctg

SEQ ID NO: 75 - DNA, 401 bp, homo sapiens
  variation 12: /replace=“g”
tgaaggggtggatcaggcagagggtcttaggccactgctggtaaatgccaaactaactctgaggtcagaagtccttgatgctacatccccaggttcaaaagcctaagaagccctcatttggactgggcagcgtccctccacctgagagaaattaataacagcaagcttctgaataattgctgaaatccacacctgaacccggatggaccagagcagccacaaagtcctcaaagcacctgaccagccccctcagtgggctatttttgtagaaaaagaatgagtcctggaaaacaaccaaatacccattgtttggctgcgtttattttccatcaagagtgatgcagatagcttgcccagtagggacccactcagcgtctaaagactaggcaaatggaggtggg

SEQ ID NO: 76 - DNA, 401 bp, homo sapiens
  variation 57: /replace=“c”
cattcaaatacaggaataagaacttcattttgtaacaaaaacaataattatataacacttcttaatattagctatcacttcctaatattttcactgtcacactgagctaaattttaatatgaattatctcactcaatccttaaaccaatctgtataaggcaggcaacataacttttccaaagttgtacagggaacaagaaccaaaactaggacccaaatcaaggattctctgatgatctccatgcttgtaatgttctctttctttttcccccaccaatatgtggaccacggtcttcttaacaaaatagctgcaatatgtggtattaatcattatgtacacacacagacacatacacaaatacaaaggagatttgctattaacttagattgtacaattaaaa

SEQ ID NO: 77 - DNA, 401 bp, homo sapiens
  variation 240: /replace=“c”
cttgattcaaaacggaatgtcatgttggtcagcaatatatgcttccactaaataaccaaatacacaaattgaaataaagtgaaattctttttccagagttaatgaaaggttcctgtttgtgaatttggggctttgaagtccaaggcttccaaacagcaccaggaatttactggtgatttattactatttggacaagtggcgaacagagcattactctacggaaagtccacattccctacatgtcaagggagccttcctgaaatggggacaccagctccccttgctgcaactcctcttagggctgtgcctcaggtggggtccacatagtctggtgtggtcatctctcctgggccagcgccctccccctctctctgctctagcccctgccctatcctgtgtcg

SEQ ID NO: 78 - DNA, 401 bp, homo sapiens
atggaaatataagctgtaaactgaaataatatattttctataataattatgaattcagctggatggaacagaaaacttaaatgacagcagctattctttcccatacagcaagaaggtgagtggtggatggctgagggccagcgcagggctcctgatgttataaatgtccaggttgcctccctcttcaggctcggcaccctaagttggagacttttgctctcagtatcacaggatgggtttccaggcgttgagactgcattccaggtggggaaaagaggaaagagcaaaggtaaaaattcagctgaatctacactgcttatctggagaaaaatacttccccagaagctccatttggtacatttctgcttatgtctaattggacagaactgagtcacatggcc

SEQ ID NO: 79 - DNA, 401 bp, homo sapiens
  variation 274: /replace=“t”
ttaatgcttccttttgtgggaagaacccctgaggtaatcagtggggttcactgaggtcccctcctcttttcctctacaccactgttttatgcgtcctgtctagcctcgtgttgaaagtgcctatttctgggctccaattaggctcattctccctcttttctttgaattatcatgaactgaattgatctgtcttttaaaaaattatccttgtaaattgggtctatcttttaagtattaaccttatttccctcaattcaacttatttccaaataactattaggcctgcccattaggtgataagtttttttaaaaataatttgtcactgtagaaatattcaaacatatacaaaaatagagagtatagcctgtgaactcctacatccctagcacccagctttgtc

SEQ ID NO: 80 - DNA, 401 bp, homo sapiens
  variation 15: /replace=“t”
ttatttccaaataactattaggcctgcccattaggtgataagtttttttaaaaataatttgtcactgtagaaatattcaaacatatacaaaaatagagagtatagcctgtgaactcctacatccctagcacccagctttgtcactcctccatattatgttgaagtaaatctcagatatctttctactataaatatttcaccatattcttttaacagctaaggaaacttaaaaaataaccatcatacaatttattacttaaatttattaatacatttttaatatcagatggtaagttacaggatggagctgtgacacccttgtcatgaacaagcctacagttgtagacttatgctcacaaagggtgctcagctccctattaatgaattgattgatgaatcaa

SEQ ID NO: 81 - DNA, 401 bp, homo sapiens
  variation 117: /replace=“g”
ctgtccatccgaatcccaaaagctttccttgtgcttcctgctctgaccaaagaggcatttgaggcacatgtgagagaacagctttccagaaaccacacttgagtccacagcagagcagaaggggcaggaggcatcaggaccatttccagatttctctctggccctgattgtctagatttgcatccaatcactgcaagagtttatacaaaagttgttagaaagatttgggtttcacacagagttatgaaaacaaggtagaagcctgatgtcttcatttcccatgaaatcttgtgggtgtaaatgaaacatgttttagtctcaaatcctggagctgtggggttcacaatggagaaatatccaggtctcttgaacaagactggacgataaccagggacagttcc

SEQ ID NO: 82 - DNA, 401 bp, homo sapiens
tctcactccatgtcaagggctattcttcttctctctctctgtctctaacaatattgcaacgatattctgaatttagttgtaaatttagttggaatttgatataagctatttgattttccttcacaaatttgtttacagcaatttcctcccagaacacactgataaagagaagtagatgacagttattgatgaaccctgcttctggcagttattaatcagccattgacttgatgaagcagccatggatgccacacctgatggtgaggcatgtgacctggggtccagctgccatgggtcatccatgtggctttgggagtgttgggaagctactgcttcagcatctccaaggaacaagcgtcctgtgagagtggaagtgagaggtcagtaaagacagacccgtt

SEQ ID NO: 83 - DNA, 401 bp, homo sapiens
  variation 101: /replace=“g”
catatgtcaatgcatagaaattatttcaaataaagtatgtttctgtgaacataaaacatttacttgttatgacactgtaatcagggcgtcacagattctaagatatgaaatgctaagcaccccatctctaccaaaataaaaaaaatttagcccggcgtggtggaacacactgtagtctcagcttctcaagaagcaggggtaggaggacaacttgagcctgggaagctgcagtgagctgtggttgtgtcactgcactccagtctgggtgacagagggagactatgtctcaacaaacaaacaaacaaacaaacaataaataaatagaaagaaatactaagtagacatttgattatctcatgcccaggttgtatctggagataagggtatgctggaaagaattc

SEQ ID NO: 84 - DNA, 401 bp, homo sapiens
  variation 110: /replace=“t”
agaatcttagtggtgactgtgcaaagccatttggtctaatgattttaccaagagcatctcagaacactagtattggtagtatgtagccttgttaataaatatatacaagcacatgttgaaatgacttgttattatatggctagagcaaacccacgtcatcctcatgatgtcttgctgtcacaatctcattctctcttgggttctgacctgactcctcaagcaagagaacaagggttggcagagctctccaggaatgcacagccccaggtgcccagcagagggcatcaagtatgacagatattggaatgacatctgttgtgtgtgcagagcagactgactgagtggaaccatgaccaaagagaactcaggtagactcaagtgcttctgggaaatttgagaaa

SEQ ID NO: 85 - DNA, 401 bp, homo sapiens
  variation 75: /replace=“t”
tgttattatatggctagagcaaacccacgtcatcctcatgatgtcttgctgtcacaatctcattctctcttggggtctgacctgactcctcaagcaagagaacaagggttggcagagctctccaggaatgcacagccccaggtgcccagcagagggcatcaagtatgacagatattggaatgacatctgttgtgtgtgcatagcagactgactgagtggaaccatgaccaaagagaactcaggtagactcaagtgcttctgggaaatttgagaaacttccctgacagagaatttacttcaagtcagatacctccccaataattgaaaatcctgccaataatgaggaaagagaagtggacccaaattcctatttccctgaatctgctcttcagtttgttcat

SEQ ID NO: 86 - DNA, 401 bp, homo sapiens
  variation 91: /replace=“g”
agggtaggggagtgtagaggttcaggagtggtgttttagtttggtgttgtgcccagggcagaccctcagataaaatgcctttaagacaagatcctgtggaatggagggaggacctaaggaggcaatgcagatgtctggtaacggagggtcccaattagaaaacacaaaagatcagaaaacctaaggcctgagtcagggtcagtacctgaagtggggctgagcagggtgttggggacgagttgaggacagagagggcctgaaggtgagatcagttatcaatcagattaggccttgtgaagactttggctcttactctggatgggaagtagaggtctggcattctctgcctttcatttggaagggaaccctcaggagtggaaggggagcaaagctgggaaaag

SEQ ID NO: 87 - DNA, 401 bp, homo sapiens
  variation 42: /replace=“g”
aaacacaaaagatcagaaaacctaaggcctgagtcagggtcagtacctgaagtggggctgagcagggtgttggggacgagttgaggacagagagggcctgaaggtgagatcagttatcaatcagattaggccttgtgaagactttggctcttactctggatgggaagtagaggtctggcattctctgcctttcatttggaagggaaccctcaggagtggaaggggagcaaagctgggaaaagtagcaataaagtacctgcaaatgttttatatagattttgttcacatctccaaaaaaagtgcaaaaactcccctagctctgcattttccagccactgtggactgaattgtgcccctccccaccaagttaatatgcagaagcccgaactcccaatgtgaca

SEQ ID NO: 88 - DNA, 401 bp, homo sapiens
  variation 78: /replace=“c”
aaacataacttatatgcaaatggttaagaataccagaaacattgcaagaatgctctttttaaatcctgaagatattggtttattttaaagcatcaaggatgatttagatttttggaaaaacagaggtggcatgaagatttctgaactcgctcacacacaaacccgtcacagccgctgtgatctataagccaaatttgtgttagaacttttcttttttgaataacaacaaaacaacaaaaacgctcctaactgaaactgggaaaggactagaaactttgttttcttactaatgtgaggttggaaacatattggctttcaagtgaattggccgtgaccacagcagcacttggatctgagggagccgagattgcaggcagatgtgagtttggcacttgcatgcc

SEQ ID NO: 89 - DNA, 401 bp, homo sapiens
  variation 352: /replace=“t”
taacatagtaacagcttacatttttatcctttggttttattgaaggatcctgtaaatgaataaaaccataagaaaacatatggaagaacagtcaaataggttataaatataatcaacactgttttaacagcatcacagagcaagcatggagggaatgatattttattgattatttagagcctaagccaggccttgtgcaacgcatttctcatgtataatcttatctgacctcatgaaggaggattttccttaaactggggagtctgaggcttagcaaatgaatcgcacaaccaggatttgaactaaagtttaccttcatctaaatacgcgtttttctttctctctctctctctttttttttttacccctatcgcatcatagccacatctgtggccacacaa

SEQ ID NO: 90 - DNA, 401 bp, homo sapiens
tgcaaccaagcgactaagggggcaggaaaggggcttccggtaagagtcgaagtgtgaattttaatttcggtggttctagggatggcaagatccaaggtgtagccaagtgaaagagttgccaaggtgggatggagagggaggtcgctggaggtgagcagcccaggacctgcagatgagaagtgggattagtggtccccagaaatgccgtgttccaaggggaagccggcggtgggcaaaggactagcagagggttcagtccgaagccgccaccagggggcgcgagagagcagcaagccgtaacagctcgcgtaaaaggcgggagctggggcttcctgggggttccctagaagcggacctcgagacaaggtcctgggtgcaagcggtttatttgggggatggct

SEQ ID NO: 91 - DNA, 401 bp, homo sapiens
  variation 202: /replace=“t”
cgagcgggatgctgtggatgacaagggagctgctctcacctccccgccccgagacagatgaggcactcgaggacctgcagcaggcctcagagagggctaggaccagggaggggctgcggatggagagcagagtgacggcctagaaaggaggttcccgagggctctggggaggcggaagaggggacgcggtgtgttgagatacggggagaagttcagagcagcatgggcatgcaggtgcctgcagaggggcccatctgggctgcaccccagcaggacagggccatggcttggtgtgggttcggctggctgcacagcttcttgcaaagtttgtccccagggtccttgggagtagaggtctttcggaaggcaccatctccagagcgtgtttctgagtaactctg

SEQ ID NO: 92 - DNA, 401 bp, homo sapiens
  variation 7: /replace=“g”
tgctgaagactgcccatgggtagcatcatgtggagtgtgcgttgtgttcattgggcctgtgtgtcatgaagacactgtgtactgttcatacagagaccctttgatgagacaccacaagagaaactccttgtaggagcctctggcagaagaaaaatggttttcttttgagctaccagattaaaaaaaaattgtggttcccctgaatcctataacagcatttgtctgcctgcaaagctcagctccaggtttgtaggccacccaacttaagcctttgtgaacacatttttgggaactgatgctaccctggacttcactgaactatcttacttcatttttcctttactcgtctttatttaatagttttattgaattatttatgtaaatctcacctcatcatgctt

SEQ ID NO: 93 - DNA, 401 bp, homo sapiens
  variation 30: /replace=“c”
caaggaatgccctagaacaaacgcctggcacatagtaagttctacccaagtatttattattataataattgtataaacatttaacgacttccttaaattgtttcctattacttgctattttgaatagtgtttcaattaatatccttgtgatatggttttgctgtgtccccaaccaaatcttatcttgagctgtaattcccacagttcccatgtatcgtgggagaaacctagtgggaggtgatagaattatgggggcgggtctttcctgtgctgttcttgtgatagtgaatgagtctcatgagatctgatggctttaaaaatggaagtttctccacacaagctgtctctttgcctgctgccatccatgtaagctgtgacttgttcctcctggccttctgcca

SEQ ID NO: 94 - DNA, 401 bp, homo sapiens
  variation 54: /replace=“g”
cccccagctcttatcacagacactatacaaacaggaatactttgtgttaccctatgttaatggttctcttattttgctgtgcatcagaataccctggaaggatgactaacatacagattgccaggctcagcacctgctgtgtctaagccaggatatctaggggcatttctagtaagttccaggtaatgctgatgctgctgatttagggacaacactttgagaaatatggctgtgtattatgggtctatttgaacattcgaggaaaataagtaggataaaagtgatttgggacaacagtctcattcttttgatatgattaatacatatgacacaaatatcttctttggtaatttttaaaaaaattagatataatagaaatttatttttctcttacacaatgg

SEQ ID NO: 95 - DNA, 401 bp, homo sapiens
ctgtcactgtgattttccttgcattgtcaattggctacagtattcttcatgatttctcttgactaacatgccttctgacaatgttgtctgaagctgtggcttcatttgggaaatttctgtgtgttttagaaaatgtaatgctgtcatcacaatgggccatagacatttcagtctttctaggtgttctgaaggggttggctcaaaaatacagggcaggctttcacgttggtctacttttagtagtgacatctttcagatttacctaaaataatggctaatatttgtctaaaaactttctagtggagaactgtactcatactaatggctagtaaaattaatggcttactgtgtgtattgcatgctcgctgaagtgttttctgtgtgtccactcagttactatg

SEQ ID NO: 96 - DNA, 401 bp, homo sapiens
aagaagccacaccttccatttctcactgcaaagagctgtggtgacagacatgtaagaattttattttctttttaaagtaacttagattacagtagggctgccatgagaaaggtgacacagaagagcagacgagagggagcagaaaggtgtatgcagattctcttgtgtccatcagccttaagaacaggggctgctgagccaagtcagaggctgaagccactcctagggataagatccccccatgtctctactctggggacattagctccactgtttagactagcttctcactttcacccacatttttaggccagtggaaacttccataaatccaggtcatacattcacattcaaccgatgtgtgtttacccactacttccgatctgaaattttgggaagct

SEQ ID NO: 97 - DNA, 401 bp, homo sapiens
  variation 48: /replace=“g”
ttccccagtgggacgactctgcggcatgtctgcgctggctcccagggaacagcaccccagatgcccacactaataacctggtctctcactcatcctgactgggctgccttcccttccctgtctgtcttcccctttcttctccctctcctcctgggatcacctcccatacaaactcctcaccctcatttccttgtcccaagttctacttttcagggaacccaggatactttcctcctggtcctaagaaaacccaggcaaaccaagtttcctgctctcatacaatttacagcatgttgggttgaacaggcaacttaacaaacattaaaaacgaagtgtaacgagtgctgaggtcagggagcatagactggcggaaggcacagagccacaggggaccctaagct

SEQ ID NO: 98 - DNA, 401 bp, homo sapiens
  variation 11: /replace=“c”
ttccctgtctgtcttcccctttcttctccctctcctcctgggatcacctcccatacaaactcctcaccctcatttccttgtcccaaggtctacttttcagggaacccaggatactttcctcctggtcctaagaaaacccaggcaaaccaagtttcctgctctcatacaatttacagcatgttgggttgaacaggcaacttaacaaacattaaaaacgaagtgtaacgagtgctgaggtcagggagcatagactggcggaaggcacagagccacaggggaccctaagctagcccaggagaggtcccagaagctgagctgggagtgactggggctccctgccttttcttccccttcttgaacttccactcctctctgtttcatttcttctcttcctttcagaa

SEQ ID NO: 99 - DNA, 401 bp, homo sapiens
  variation 83: /replace=“t”
tttgggattctggagtctattgaatatttgcaacttctaggggaaggcttggatggtaaattgtttaattttgatcattttcagctcctagcacagtagctgctacctatctttcacctctggaccctcctctcttgagcaggcagcatgcatgtgttcctggagaagtttgcacacagcttgttggaggcagggtgggcaaaaggatcctgccctccaaatatcagggatttgtactctgactgctgatggctgcttctgatcacaggggtgctggcaatgaaaaagccattattgttgttgcatcttcccacattattgcaagcccgtctacctctggatgaactgagttccacggtatttaaaaagctggcacctttgttacccacttcagttttcta

SEQ ID NO: 100 - DNA, 401 bp, homo sapiens
  variation 26: /replace=“g”
agggtaaaaacaaggacatttcttaagaatctatagcagtaatccaggaaaaaaataatgtggtcttaaattggaaagtggtgggggtaagatatcagattcatgtgatcatgactaagcaactccaggattttctgacagaaagcatgtggcatgaaggaaagagcagagtcaaggagggctctggaacttgggccagaaaaactagaaggataaataaataataaaaagtttagcagtcaaattcataacagtaaataaataaattaaaaataataaaaattaatggataaaaagttgttatgtaaattcagtgccctattccatctcctaggttattttcactagaagttaattagaactctccagttgtcccagcctagacagcctgctagcccctt

SEQ ID NO: 101 - DNA, 401 bp, homo sapiens
  variation 290: /replace=“t”
aatgttatgaatatatttataacttttatagcaaataaaataataatgcattcagattttgacatggatgctaaacaaacaatgaaaggtttgtgtctgagagaatcagtattaataattatttagtattaagtaagcattgttaagtaattaagtactaagaattgagtacaatattttaaaatgtgactttacagagaaagagagtacaaattgtataaaattcaagaacacataaaaccaatctatactgatgggcatcagaatagtggctatcttttgggaggtgctgactaggcacacaagatgccttcttgggggttgtacatggcctgcatcttgacttggtggtgattgattacagggttgtgaacatatgtaaaaacttcatcaaggtgtac

SEQ ID NO: 102 - DNA, 401 bp, homo sapiens
  variation 265: /replace=“t”
aatcaagtaaaagaatggtttgaataataacatattgataacagataatacaaacttttttcattaagcttcagaagaatcttagtagaaatgcattgtttggaatgcatatgctttggtctcaataaaaatatccggttgcagagatatttcatgtggtttgaactaacaggcaaaaaattaaaaattcattggtatacactaattcatgcaattgtatattttggaggtaagtgtaatggcataggtttgctgtaatggaaccgattggaaaatgatctcttggagaaaatttattgctggtcataagatttctttggtttagaaaaatgctgggtttagtaaaatctgaggcactaaatgaaagtctgctttaataaaaatgtggatctgagaactgt

SEQ ID NO: 103 - DNA, 401 bp, homo sapiens
  variation 26: /replace=“g”
gctctaactctggggacttgtattaagccctgactttgttctctgtctccttgaagttaggaatccactgcactgagggtgccaaagtgctcctcaatcactggttcctacagtccaatgggcaatatcagcaaaagtgttttgtagtgtagtgacagtgggattcattctctttcacatatgtccacagcagtggtggcagcagctgcagcaaagtgctagtgggtgccagggtgcttgcccctctgcaggtgttcagcaccgtggtggaggcaacacagctgtggggggcaggggcccttgctgatgactgtgtgcatggtcatactggaggtggtgttggctcaggggtggggcactggcagttgtagttctgggtgccttctctgtgccccacaagc

SEQ ID NO: 104 - DNA, 401 bp, homo sapiens
  variation 249: /replace=“t”
aattttgtttcctggaggcactctgatacctattactgtatcaatcaggatccaggcagaaagtagacatcatgcttaaactgggtaacttgaagataatataggaaaatagcaagcaatagtctagaatcctaaaagtctgaatctattatcaaccttaggtgtgaatgggcaaggggagggagtggattctgtcatacaacacatgagtgcaaaagagctgtgtagtgctgcttgatgcctattacctttatgtaaagacacagccagctcacgtgagctcttaggaagggggatagagaaataaataccctgacctcatcttcctgccctctgggctgctggcctccaaaccagaagcagggcactggagcttaccaacatggtacacaaaggctggc

SEQ ID NO: 105 - DNA, 401 bp, homo sapiens
  variation 85: /replace=“g”
gtgacacattgaattttattactgactctgtgtttcaaaacattttggggactgaaaaacccaccaggcaaatctcaggccttaaaaagcagttaaactgaattgcaaataaaaggcttagaaagtccttaaaatcataaacttagcatctccaataggacaaatgaactttgtcttctcatctgagttaacagatttggaagaatcaaaatcttttctgttgtatagactacatgctaccatttccacaggcaagctgcccctaaatttggaaaacttttccttctgaaaccacccaatctctttagcttgatttgggcaaccgaatctcttaggcttttgcctctttgtgtccaaaactagcactgttcctgagagcaggctatcaatcatttacatat

SEQ ID NO: 106 - DNA, 401 bp, homo sapiens
  variation 38: /replace=“c”
gcgtcgctcacgctgggagctgtagactggagctgttgctattcggccatcttggctcctccccaaggggaagctttttaagctttttattctaaggactattgaggtcatttcagtgatcaaagtgagatttattgttcactaatcgggctcctatgtgatcttatattgcatattagtgcagattttatataatttctattcacaacagctattgatgaaacaatgtttcccacatttttgtccttttagtgctaaaaattaaaaccaaagttaggggtatagaatttaggattatagaactagggatttgttggtccttaaatagctgggtgctgtttgttgaaagagtgattctactatgaaattgcatagcatgttgcaagttccatcataatata

SEQ ID NO: 107 - DNA, 401 bp, homo sapiens
  variation 198: /replace=“g”
aactaacctcattctatgcccacttaacctttctaatagctttagcactgaatgactcatatgaacccattttacaggtgagaaaattgaggcctagagaagttttgaggcacatctgcttatagctagtgaaaagtgaattaaaactctgaactagtctcaccttatagccagagctatccatcatggtagcacgcagcatctaggtgagtttgtgctctggagagttcatgtttttcactgctcacagctcctgtgttctaggtggtacgtaatggtgaacagactgggcccgagattaagattacatgattggaagtgcaagttcatcacttttggagcaccttgggcaaatcacttgtccttcatagtcatttgtttctgcataagtaaaattggga

SEQ ID NO: 108 - DNA, 401 bp, homo sapiens
  variation 42: /replace=“g”
gtctcaccttatagccagagctatccatcatggtagcacgcagcatctaggtgagtttgtgctctggagagttcatgtttttcactgctcacagctcctgtgttctaggtggtacgtaatggtgaacagactgggcccgagattaagattacatgattggaagtgcaagttcatcacttttggagcaccttgggcaaatcacttgtccttcatagtcatttgtttctgcataagtaaaattgggataataataatagtggataatagtgctggctcatagagttattgggagttaattacatgagttaaatcatgtaaagtgttgagggcagtgcctaggcacgtagtaagaaatgtgagtgattactgtttttataaccttattgttaatactaatttgc

SEQ ID NO: 109 - DNA, 401 bp, homo sapiens
  variation 139: /replace=“g”
atagtggataatagtgctggctcatagagttattgggagttaattacatgagttaaatcatgtaaagtgttgagggcagtgcctaggcacgtagtaagaaatgtgagtgattactgtttttataaccttattgttaatactaatttgctccagatggcatggtatacagtggtaacagctgatgacttctctccatttgaataaactgtgctaacttaatacagtggctctctggagcactacaggtctagccaaccctgcagcacatttaaggtctcagtttacatcacttcctccagtatctcattggccaaaccaagtcatgtggccaatctcatcatccatggaagggggatcatatttgtgccattaagtttcatgatagagagaaagagggattg

SEQ ID NO: 110 - DNA, 401 bp, homo sapiens
gccttggtctcccaaagtgctgagactacaggtgtgagccactgtgcccggccagtttttcttttattctctgtctgcttccttcatttctcaaaggcatctcaaactcagtgggcccaaaaccaaagtcaagctccatcagaacatcctgctacttccaactttaaaatatatcttaaatcacaatttcttgctacatgcaaccctttggttcaaactaccatcatccctcgcttgaacttagaattgattttcctgcttcagtatttttttaaatgatgaaatatttcaaataaaccatgtgtggctttatactttttattacataaaaaggatgaaaatctgccgggtgcactggctcgcacttgtaatcccagcacccatcccaggccgaggtggat

SEQ ID NO: 111 - DNA, 401 bp, homo sapiens
  variation 183: /replace=“g”
tttattttcttcaaaacattaaagggaagaactaaaaacaaaaaccagcaaacaaaataatatttgtgaaaagtgattttcaaaaatttccaaactgctgtggggtgatgagtgacacacatttccataaagtttctcattctatctgtgtctttcagtgtgaacaaaaaatttctttctaaaatgtgaagattttactcacttcactactacctcctttagtggttggaagtcatttgaatttttctttacttttctagcaagaattctttgttttgtgctatgactcacgtgaataatagctcctggcttaatctgaagctctgtgttggctctgccagcatgagacaggaagaccttctccacttttgcccagtaattttcattctccccccacaggg

SEQ ID NO: 112 - DNA, 401 bp, homo sapiens
  variation 18: /replace=“g”
ggggccaccatctgtgtatatttgcacaacctccccataactgcatgggttttctctgagtactctagtttgctctctcattccaaagtcatgcacattaggttaattggcatgtctacatggactcagtgtgagtgaatgtgagcatgggtgtgaatgtgctctgccatgagagactgtcctgttcagggctggttcccacctcactccgtgagctgctgggatgggctctggccacccatgaccatgaactggaaataagcaggtaaagaattatcttacttggttttattaatctttcttaaatgtatgtatagttcacatttatttcaatgtttaatattagaaatgttttgcctttatttagaagtgtggtattttgtgatcagaaatctgccata

SEQ ID NO: 113 - DNA, 401 bp, homo sapiens
  variation 6: /replace=“t”
agagactgtcctgttcagggctggttcccacctcactccgtgagctgctgggatgggctctggccacccatgaccatgaactggaaataagcaggtaaagaattatcttacttggttttattaatctttcttaaatgtatgtatagttcacatttatttcaatgtttaatattagaaatgttttgcctttatttagaagtttggtattttgtgatcagaaatctgccataggaacttcattatagtttatatcaattagcctatggaaaaattgtttttgttatatgtcattttgcttaaagctgcagtttccaggactctatcaacaacattaagtgaggactttgtgtgatttgcggggccaagtgcaaaatgaaattgtagggtaccttgttcaaaat

SEQ ID NO: 114 - DNA, 401 bp, homo sapiens
cttaaagataacaaacatatatgtttaacattttgagtttttaatctttttttgtttagaacctctttatggaaacatgtctagcttactaaggtaaaattgactcactcattgactcatttagcaaattatttattgatctcctatttatcagacagggtggaatatagaaaaggtctctgcccttctggagtttctaacccagtgtggaaaactgataatacataactaaataactcatataccagtgtaattacagtggataagtgctaggaagaaaatgattgtgatagtaggtaaccaggaggggtgtacttggataactatcacctcttggatcttgactcctggcttgatctccctcttagagataggcaatgcaagtggttttttgtattagc

SEQ ID NO: 115 - DNA, 401 bp, homo sapiens
  variation 128: /replace=“c”
tctttcttagatgatctattcaaagtgtggttatctactcactatttttgtttttctctgtgcaggaggtaccagatgcctctagtcagccgtcttatagcccctcctatatcgttttaatttttgtattggtcaaacttgcaaaaggttgtctactttgttgatgttaaaattttttcgttttatcgaattttgattttctaattcacacttcaggcttttatatttatccattctgtccttgtagcttagttggatttattttgttgttacttcctgtgcttctttgtttgacgtattagtttacttatttacatccccaccagcaatgcacaaaagttctaattcccccacatcctaccaacacttgttactttctgttttgttttttttaattataa

SEQ ID NO: 116 - DNA, 401 bp, homo sapiens
aagggtgtatcacaacactgggaagcatattccttaacattaggagtttatgtgatttagctgaagagagaacacctgtattagttaaaagggaatatttagcagcatttaaacaaaagctatttcacatgtcactaagtatactataagaatttccagaaagaagtatcatcctttgtctaatagttcagaggatttctattagtaaatgaaaaatgacagcctcattttatcctccaaatatgtcaaaattatcttgagttgttgggtaccagaactgataggtgtcatgtatttaagcccatttgaaaaatacatttcaataatggagtgagactctgtctcaaaaaaatttcaaatgtcttttctcaattgaaaaaacctattatttctagatagat

SEQ ID NO: 117 - DNA, 401 bp, homo sapiens
  variation 39: /replace=“t”
cagtgccttgatcttggactgctcaacctctaaaactgggagaaataaatttcattatttataaattacccagtctgggtatgtttttaatagcagcatggatggacaaagacagtgtgtaacactcagtatcaacaaactccaaggaaatgggcaccctgatactctattgtgtagtgtaaattcacacaaattttggaaagcaatttggcaatatatatcaaaatcttaaaaaagttctaggcacaagaaccctgttttagaaattaagtctattggagtagtctgcaggaagttgtgtctgtaaatttgtttatcacagttctgtttatactggaagataaccctcaagaaatgttatatatctaataatagaggattacaattacatgatgaaattt

SEQ ID NO: 118 - DNA, 401 bp, homo sapiens
tttatttagttctgctctgatctttgttatttcttttcttctgctgggtttgggtttggtttgttcttgtttctctggttccttttcagtagttaggggttaaattaaactcagaaatcagtctatgcataccctgacctcagagatgatgctgttccaccacacctttgctgggcttgcattctactttggaattccacatcttgagaattatctctttaatggcaaaagtgccaataagcctttcccaattttctccattgtgaaagtaacatctcttatatcataatgtatataatatccatgtcaatgtattttgggtgtaaagggtgaaagttaggaagtatccagttggaattctgtgtaaagtgcactgaatctggggaggcagagaatgctct

SEQ ID NO: 119 - DNA, 401 bp, homo sapiens
  variation 1: /replace=“g”
agtttatttaccttttctaggattttgggaaaagagaagggccttgcagagcccctcttattaataacccctgcccaacccccctacttttccagatcctttccctttctaaggagaggtcctgcaggacgcctaccagagccatgcatcagacagcggcttctgcccagtcacggctgggtgggtgtggccagcttatgagaaagttcacgtaagaggttgatttaatctctccagttttaaaatgtggaatggtatggtctgaatggctttgaattagaataaggtctaaactgatttgagagcaaggcctggatttgaaagggagagtgatgaaatcaaagaatgtcagagccaagtaatgttagagatttaacaagaactgcatgtattccctgtta

SEQ ID NO: 120 - DNA, 401 bp, homo sapiens
atctggcaaaagaaagtcattcagattaaaaaagaataaattaaattatccctctttgatgatgacataatcttacagctagaaaaacataaagactccaccaaaaaactcttggctccgataaataaattcagtatagttgcaggatacaacatacaaaaatccatacatttctatataccaataataaactagctaagaaaggaatcaagaaggcaacttcatttacagtagcaacaaagaaaacaaaatacctaggaataaattaaccaaggtggtgaaaatctctaaaagtaaaactataacacatggataaaagtaaccaaaaagcacatcaacaaatcaaaagtcaccgcatgctcatggatcagaagtattcatatcattaatgtttctatact

SEQ ID NO: 121 - DNA, 401 bp, homo sapiens
  variation 328: /replace=“c”
ccaattgttttctagcaatgaaaggaatcatgtaaaaccacactagttaccttaatcccagttagaaaacactagaggaattcgttatgaaacccaaagacatgatgaccttcacaattctccaggcaagttcttcatgtaccatgacaagtgggactgagagttttgggaaatgcagaagaaatgtgtcattttgaagaattctatgtggaagattgggattctagtggcttaaaatgatgtggagtgaaattgttttcaatgaaaatatttatcaaagttatatcatgagtatatctaaccctatgtttactgtatttgtgctatgatatgaatatattttgatttagtatttgatcagtctttactgattgttgagaatctactaactcagtgcttct

SEQ ID NO: 122 - DNA, 401 bp, homo sapiens
  variation 16: /replace=“g”
gaaaagtaaagtggaagtaggcataaagatgacactgcaggtctcaattggagattcaaagtcagtcatttccccagttaggctctaacacaatgtaacatgatgaggggagggatttgctaatcacaaccagagtcaccactgagtgaaggcatgttctatgccaggctcccaaattaactaccctcattaatttcccaatcttgcttttggggagccacaccccacctcccttgatcacactagactgtagcaaagacagatgataacaataatggttaatatttactgagagcacaccaatgccaggcactgctctaagccttttgtgtgttttaactcattttattccctacagtgaccttctgaggaagtacctattatgatctttatttttcaga

SEQ ID NO: 123 - DNA, 401 bp, homo sapiens
acctcacccagctcccatgcaaccaaaaaggccagtctcactcccactgtgcgcctgccaacagcaccaagtttatttccaggcagtgggtaagcagtgctgagaacttgccccgggctaccagcctcccagctgagagagcaggcaaggcttttgcgcttccctgcctgttgagtctgcccacaagattcacgccatccactgagttctggtcaggaagcttcatgtttggttggaattattaccaagtttgtctagaggtttccttctccctgtggtcttttccaagttcccctggtagccctccccaaggacgcctatgagacaagtcagaaatggcttccctggggacccagaaatcccacggggcttttcctgctgcttcctctaccctgtatttt

SEQ ID NO: 124 - DNA, 401 bp, homo sapiens
  variation 182: /replace=“t”
aacagaacaaagacataaaaacagataatggtatgagtactgcagttgaaatgcaattagggattctttagcctacttggtctttacacctcttgccctgttccccatccctatgtggcatcacaagaaatcttacagcctgaaaaatatggaacataaaacactgttctgtcatttatcacacaaacagggtcaatttctagaataaaagaagacattcttaataaatttattctgggaaaaagtcttaaacgggtgataccaggaaaattgggacatacggtcatcctggttctataccacaagagttgaaatgagaggaaaatatggttaagtgagaagaaatggattccagccccatttctatcatccacgagctgtgtattcttaaaaagttactc

SEQ ID NO: 125 - DNA, 401 bp, homo sapiens
  variation 81: /replace=“t”
atgactggcacccaaataggcttgctgggttggtgcactgtcaggtttccaattctttcgatgtgtctgtagccttggtcaggccaggtctgccttgtcctcctggatggacatttaggaaatgcatcctaaccccatcagcacagtcatgcactggtcttcttttcccatatgtgtgtgccgccatctgggacaacttacccattgtctgctagggcctttgatgcttgattatagggaaagaggaaaacatcagctacaatagagcaatctgttctgtttgggacatcttacaatcttttttcctccttttctttttttcaatgcacatatttttgatcatcttcacatagggatgtatattggtaatggaatgtattctagaattctttctcttgaaa

SEQ ID NO: 126 - DNA, 401 bp, homo sapiens
  variation 348: /replace=“t”
taaatattatatgctattaattctaaggtcctgacataatactctgccattaagcttttcatctggaggttgtatattgttttgtaattatttgctaattaatcaggcactagttctagaatttaggaaacaccagctcagtagcttctcaaccatgatgaagagtagacagtattagaaaaacacagtagtgggagagtagaagattgggggaaggaaaagagagtccctaagcacaaagtgtgcttctctacctctcctccttctccattctgtgcttccctggcagatcttctgggcgaggagccaagagaaggtttgaaagtttgtctgcagctagataaacaactttagtggagagatggattttggcgtcacttaggaggtcccacctaattttg

SEQ ID NO: 127 - DNA, 401 bp, homo sapiens
  variation 56: /replace=“g”
ttcaattttgtcagtgtttgcttcatatacttgaggtgttctgtgaaagggctgtatatacaacatgatctccaaatgcagattgagccaagaaatgaaaaaatgagtcagacaaatccagtttgtcagtaaatggtgatttattagaggacttacagacagaaatgtggtcttgggtggctgcaagccaggaagaattctgcagtgcaaccctctaggcccagggcttatatatcttgggtggggggtaagtacatatccctggaagaaatgtgtaggtgacttaaagaatactactggcatcacaacctatgatgcatccaacatcaagggctgttttggaggaaaggtgaaacttacaataaataggtatttctacatagggagtaatatatcaatga

SEQ ID NO: 128 - DNA, 401 bp, homo sapiens
actttcactttactccatttctaagtttgatattctggtagaattaaagtaagagttttctttctttcatctcctttttatctctctcatattgatttcttttaatggaaaatttttttatttattattttatcgttccgatagactttgccgagcatctctcctcaaatctgtatccagattatttcctcaattgtcttaagttggctgtgctatatcattagtttatgtctttttatcttcccatgtaaatactttactaattctgggaccagaatagatatctttgtatgtgttttagttagctattgctgtataattccagtatccataggcgcactttgttaaagagtggctcatccgtctatggtcacatgaaaggaagtacaattcagggttct

SEQ ID NO: 129 - DNA, 401 bp, homo sapiens
  variation 157: /replace=“t”
aaacattgtaatttaagaatttcttttgaaatttttattttcctctgccagttaattcagtgttattttattttaccagttgggatcaataaatctttgttacagtcatcaaccatcactatgataaattttgaatttcatgagctactcactcaacatttttaaaattatttcaaaattggacacttaagttcatctataagctgggtataggaaacagttaagaatgtgtaggtatcagtgcaaactgtgtcttcctgtacctagaaaatcccctgaatctttctcccattccaaggacagaatttctttttcctactttgcactttttccttctctttaattccattaaaatgaagttgcaatctctagatggcagatatttcttaagcacaaaaact

SEQ ID NO: 130 - DNA, 401 bp, homo sapiens
  variation 387: /replace=“g”
ccacagcgtgtgatctctttgcacatgctcacccccacttaaactttccgtcattagtgaaaacatcatgaggccattaccagatgcagctgcctaatcttggacttttcagccaccagaaccatgagccaaatatacctcttttctttatatattacccagcttcaggtattctgttgtagcaatacaaaatagactgaaacagtgtgtgaatgtggaaaagtctgggaatgtgcaagtaggcagtaagatgctgtctaaccaaagcctgaactgaaagggcagtgatgggtatttctgttttcccaaagatttttcatatcatagtgtaattatatcaacaagcacccaaagttttcagaagtttcacaatatgcctacattaaagcataccagattat

SEQ ID NO: 131 - DNA, 401 bp, homo sapiens
  variation 46: /replace=“t”
cttcaccttctcaggtaatacctgatagtccaccaggcagacggggttggcgatgcttccagcactgaacactctctgggtttgctagaactacccaggccaacaccccaccacttttttttcctctcatttttgtaatgtggcttcttataaatttaaacttgtttcagcatttgccttgttgcctgtctggaaataacaagttgtttgttttgctttataatttgagaaacaggctaccattcccttcccatcatggaaaacattctgcaatgatctgtatcatcttccaatcatggtaatcactgactaacctgttttcaccagagaggttttttatctaattggaagtttgagcctgaaactcagagaagataaatggacaattttggtctcacgtg

SEQ ID NO: 132 - DNA, 401 bp, homo sapiens
  variation 141: /replace=“t”
tttttactccactcttgctatcttacctcctgctttttttttcagtgtatgtctcctctggtcacatgattatatttatggggtgcacttgatcctctactgtgttattttctattcctacatgatcttatttagtaccggggctttaaagaccatctgttgatgactcccaagtcagtatttctggctccattctcctcaaatggtaaactcaaactgaatatctccttttggatatctgaaaggcatctcaaatccaacatctcaaacattactctgagttctccaccccatatgctcctctaaaatctttcccatgttattaaatggtcccaccttaaacacacacctaagagcaattcttcactcttctccttcccaaaagatctacatctaattta

SEQ ID NO: 133 - DNA, 401 bp, homo sapiens
aaaaataaaagacaaaagtaattaagtggcaaataaaaagtagagtggatcgttaagactaaacactatttttaaagaccagtataacagacaaatgttttgatggtgaaagagagtgagagtgagagagagaaagtgagtgagaaataatataaacaaacaatatcaggaaaaaataatggagcacaaacatataatgaagtattttagaaactatgaattgatatgaacaactgtatgccagtaggttaaaagcccatgcaatggacaattttctaggaaagtgtaaatgccatgattagtcttcacaaccataaaacaagtattaatacaaattgaaatggtaatcagtatcttattcccaaaggcatcagacccacatggtttttccagtaactttc

SEQ ID NO: 134 - DNA, 401 bp, homo sapiens
  variation 5: /replace=“t”
ggcactattcccaatagcaaagacttggaaccaacccaaatgtcgaataatgatagactggattaagaaaaagtggcacatatacactatggaatactatgcagccataaaaaatgatgagttcatgtcctttgttgggacatggatgaagctggaaaccattattctcagcaaagtaccacaaagacaaaaaatcaaacatcgcttgttctcactcataggtaggaattgaccagtgagaacacatggacacaggaaagggaccatcacacactggggactgttgtggggtggagggaggggggagggatagcattaggagatatacctaatgctaaatacgagttaatgggtgcaacacagcaacatggcacatgtatacatatgtaacaaacctgcac

SEQ ID NO: 135 - DNA, 401 bp, homo sapiens
  variation 323: /replace=“g”
gaatccgggaggcagaggttgtggtgagctgagattgcgccattgcactccagcctgggcaaaaagagtgaaactccatcaaagaaagaaagagagagagagagagaaggagagaaggagggaaatattatgaaaaaaagtgagacagagaaaaccatacagaccagagtgaaacgcagttaaagctaatccgtggtggtagaggtcagaatatagataccttgaagactggctggttaaggaacccaaggaaaccttttgaatgtttcttgacatggttgcagattacatgagtatatacataagtaaaaattcattatgcattttatcgtatgtaaactacacctcagtttaattgaaaaatagatcttatatatcttttattatgtttattgacttga

SEQ ID NO: 136 - DNA, 401 bp, homo sapiens
atttacaaaatatatcttttcccttctttaaaaaaacttcatagtaaattatgcataaagcttaccatcatatcatttttaagtgtacagtttactggtgttaagtacattcatgtccatcactgccactgatctccagaactctcttcatcttgcaaaagtgaaactctatacctcattcctccctccatttatgccacatcaaaccaccattctattttctgtctgtatgaatttgactattctaggtatcttatataagataccttatataaggtaccttatatcttatataatatcatctatattatataagtggtatcttatataagattccacttatgtggaatcatatagtatttgtccttttgtgactggcttaattcacatagcataatgtt

SEQ ID NO: 137 - DNA, 401 bp, homo sapiens
  variation 114: /replace=“t”
aaataagaagaaaaaaaggagtcatctctgggactacaccttgagaatctcaagttttaatggatggcttaggaattactcaagttaggaggcagaaagagccagcagagaagcaggaggaaaacaggaaagagctgtgacccagagtaaagggccatagaggtcagctgtatcagatgtgaccaagagctcaagtccaaaaaggtctgaataataacccttgtgatggggaggttatgactgacccccaggggctatttcattgaaataatgggatcagaggacagtttggagatggtggggtgtgactgggaaataagaaagttggaatagcaagtatggaggcctcttcccagagaggaagaggtggcatggcctctggagccagacaatccaagttt

SEQ ID NO: 138 - DNA, 401 bp, homo sapiens
  variation 124: /replace=“c”
tgtccatatgttcacagcagagtttttgatgtgtttgattgtggggtgataccgcagaccccactgagggtataacacacaccaggtgacttcactaaaatccccacggtgaaacacatttgcggcccttttccctgcccctggattttgctaaggatctggggggttctgtggaagtctgaaggtcagtggacaggacgagtttggagatgcgcatttggaaactgtacccctcaagtgacaaggaaatgagcgtggatttaagaagataatgaccgttctgtgaagttttaatggaatttttaagcaagaaaacccttaccctgtgaaaacttcagtctgcacagaagaattggtgggagaaggaagactttgcctaagatcactttatatagcacaac

SEQ ID NO: 139 - DNA, 401 bp, homo sapiens
  variation 142: /replace=“t”
tatacacacaaggaaaccccctcccccaaacccaccagaatattgtttttacctctgggtgttgagactatagagttttaatttctttatatattcttctgtttttctagaataaatgcatattattttataattacaaaggctactttttaactcttcataatcataacttataatggtaaaatttatataaaatttcaatctgtgcatatgtaaataaattaactttcttgttatgcatttaagttagttataggtttccactgttttcttcagacaatgattatcattgtgtgtaatttcaacctagtaactcaattgtttctgtagaattgatagtagaattaattactcaatagttatggatattttaagactacataaatgctattcaacactct

SEQ ID NO: 140 - DNA, 401 bp, homo sapiens
  variation 1: /replace=“t”
cctagtgcacatgtattgcagtactagaattgttaacccatacccacatgggaaaaagtttatcaactagagtacagtgcttatgtacaatgttttttgcaatctgaggctccactgatttctaaaattacttaggtcagcatcttttttcttcaccctattcagtgaagtgtttcatccgtttgtaatacagttagattatttgtaacagtctgaatttcattttgagatccctgacctgctaaattatacttttaattggcacatattattgttcctactattcacttttttaatgaagtgaacttcacagatatgaaattaaccatttgaatgtgaacaatttagtggcatttaggacattcacagtgttgtgtaactacctctaactagttccagaa

SEQ ID NO: 141 - DNA, 401 bp, homo sapiens
  variation 122: /replace=“t”
gttgctgtgtcttcgtttttttcagataacccccaaatcagtgaataaactagttcagagtcacacggccagtaagaaatagggtcaagatctgagctcagcaaagtctgtctgcttttttctacatcataccatgcttttaagcttaatacataagacagattatttcctgcttcatttagtcgtcctatttctgccttatttagaaatagttccaaggcgatttagtttcttgcacttttctaccctgttgctgctgttttatttttcttatacttcatttcacttgaactcctcacctccagcttcatttattctcttggggtccaaaaagaaaatgattataaaccaaggaaggttgcaatgatcctttcccccagggaattttcccagggctgaag

SEQ ID NO: 142 - DNA, 401 bp, homo sapiens
cttgctttgaccaatgggatgtgagtggacataatatataccagtccaagtagagaatttaacccaagtcaaacatgtgtttggctcagtctcctcacttctgcccactgtcctaagaggagttttctccaggtaaacactactgcttcaacctgggtcccagaaagacaaacacatgctctagatctgtgtcacctaccattgccagctgacccaacacagaggacttatgaaaaatacataatagttgtaagctactgagatttggagttttttattatagagcattttcatagccaaagttgattaattcatacattaaacatcttcttaaccacaagacttccagggcattcagtttgctgatgtcctctgtagatctcaaaattaattggccctag

SEQ ID NO: 143 - DNA, 401 bp, homo sapiens
  variation 3: /replace=“t”
aggtggaattttgaatcacatgggaagtttcagtgtttcaaagcatgaagtagagcttccctaacccctgctatgactactctgctttcagtgttggaaaggaaatgatcttttaaaaggtggaatatgcaaaaggggaaaacagttctcctctgtcttttcctggggtcgggagggggattgaggcatttccccaaagatccctgctaactctgtgaccttccaagattcgttaactgtgccagagaaggaaatgtacctgcaattgctcactggggtaatatttaagtcaaggctggatttcaagctcaatttaagtgctacattttgattttttttaagaagaaagatttgcctgtaaaaatgctacttttcttatacaagatctataaacatgcttt

SEQ ID NO: 144 - DNA, 401 bp, homo sapiens
  variation 217: /replace=“g”
ttccctaatgattagtgacactgagcatcttttcatgtgcttattggccatttgtatatctgctttggggatatgtctaagtcttttacccattttcaaaattgatacgcagcagatgtacatattttcggaattcacatgacaatttgatacacttatataatcaaatcagattaattggaatattcgttatcttaaatacttttctttatactaagaacattcacaaaattatcttttagctattttgaaatatacaatgaattaatatctgcagtcataccacctgaatgcatctcatctcatctgcccattttttaattgggctacttatatttttgttgttgaattaagaaaaattagattcatagcttaattatgatgaatgtatttatcatctc

SEQ ID NO: 145 - DNA, 401 bp, homo sapiens
  variation 256: /replace=“t”
gaatatacttttatgctcaaacataaaatctggtaataaggttattattatatctatatctcagcttcatccaatttgctgtcgtctctctttattatatacttgattgatggactgatgcaccatggaggcattgctgaattgaagagaagagacatcggggtggaattaatcatgcagcagattgctacataactcttatttcccatgagatgagaatcacagtgataaaccatgaagacagctgtatgtggcctaaaactaagaccagaatgctcaaataagttccaatttatcttgtttccagagataaattagtaggaaggggtgaaaaggagaaatatttgtgaatcatacagtagtcacagctattggttgatgtctaggatattaaaatgtga

SEQ ID NO: 146 - DNA, 401 bp, homo sapiens
  variation 319: /replace=“g”
gctggcttctgagaggggagtacttattcagagaggaatagaacttaatataaacattcctccatccagaaaaaaaaagttaactgacactgggggcaaaagcctggtctccagctgtctataaaactggctgttccaatatctgaactcacagaatccctcccccaactgggagttaatttggagtttgagttgaaaacatgttcaaagttttgccctaaatcagtcagccaacaggtatttctgtgcctgtccttcgagccctgtgctgtacacttaaattttatttcttaggagccaaaaaaaaaaaaaaaaaaaaatgaaatctatagaactgattcataggtcgcaacaaaatgcacaggttggtgctcagtttcttcctggctccttatctttct

SEQ ID NO: 147 - DNA, 401 bp, homo sapiens
  variation 154: /replace=“t”
gctctaactcaacctttcatttgtttcaagagggaggtatcagaaccacaacaaatggaatgaaacctaccactaccacctgcctggcacaaagaaataatttacaattcacggggaatttcctgtgtttcattttgtctgaagaactgtaaccggacttgaatgaactaagtgtctaattgttgcattatttttcttgtagagattttagtgttttattactttctccagtttacttcaatagtgaattaggttccctctaccatctgaaatgttacagttcgataaaataggtaaattaataaagtctaaagtgcttccaagctgagcagagacctttctgtgcgctgtgcttttattgctcttcccagctgatatttataactgggccatatgtggtt

SEQ ID NO: 148 - DNA, 401 bp, homo sapiens
ggataaagtcataggaatatttttaaagcctttcatggtaaacagaagaaaaagaatactaaaagtactataattattgagcattgtgaggaagaagcaagcagaaagaaggcattgagatacctctcatagtgaaaaaccaatggatttttgtctacctttgttaaaattcaagaatttaatgagtcatttgttaaactactggaagcttgagattggccatgatgggaatatttacatcccagaaagtgaaaaaacaaacagattagaaagcaaccatgttaactaaaaacagtattgaaaagtgaggcaggggcagggaggagttagcagaaggagactgcctgttcttttgttgctgagtatgtatttttctatacatacgtaagtatttattacgt

SEQ ID NO: 149 - DNA, 401 bp, homo sapiens
  variation 121: /replace=“t”
gataaacctttatagttagaaagagtctaaatgttggtgtctgtaagcacaataagtaaatgagtttggtttttccttccaaaataactgaacgttcactttaaatataatgttagccagctttgaatccagaatatttacatcttgcagactgtgtcttgcagcctctgaatcaactgactgggagcatccgtctctgcatgcagggctgctgaaaggtgggagcagagataaaagacaggagatgggaagaaacaaagggagccaagagatttcatagaatgctgcaagagacattcttttgatctatttggactcctccaaggcgggatccagagaaagaagagtggcaggttggagaccacacccctagattagctctcctttgaactgggatggtg

SEQ ID NO: 150 - DNA, 401 bp, homo sapiens
ttcaaaaaatgtctctgcatgtcttttgctcgctttttaatggggttatttgctttcttttttttttttttgagtgtttgagttccttgtaaattctgcatattagtcccctgtcagatggatagtttgcaaatattttctcccactctgcaagctgtttgttcactctgttgattgtttcttttgctgtgaagcttcttcgtttaattaagtcccatttgtctacttttgttttgttgcatttgtttctgaggtattagtcatgaatcctttgcctagaccaatgtccagaaatgatttccctaggtttctttctagtatttttatagtttcaggtcttacatttaagtctttaattcatcttgagttagtttttgcctatggtgagagacagaggtctg

SEQ ID NO: 151 - DNA, 401 bp, homo sapiens
  variation 218: /replace=“t”
attgatagtctagaaacttgttagagagaaaggaatttaatcaaaggtagaagtaatttatagggtaagtccgacatatacatactttctctaagccttggcttgattggttttcatttttgggatgtgtatttgacatttgaaacaattttggtcaataatgctttcatgcgaggctgagagttctttgacctatacatagaaaatgagggagagccaagagaaatttggtgctggtggtgactgctgaactttcaggatcattaaaatatactgctaactgcatgaaataagcacatttgactcaagtttctccttggggatggtattggtcagtttctcaagagagaagccctcttcctctcccttaccaaagactctgtctccagttaaacagggag

SEQ ID NO: 152 - DNA, 401 bp, homo sapiens
aatttcaaatgggtcttgtattcttaatataaaccttatttggatataacatatgattcttttaatatatattgttgattttgacttgatagtattatactaaggtttttgggtgcatattcactaaagagtagcacctactattttgttttctggaattgtctttccctgatttggatattcttgttttttttaactagcgaaatgaattagtaattttttgtctttttctgttcccctgagcaattcctgtattagagggtctgtttctttaatgttcagtagtatctcatgcctaaatctgtgtgggcctagtgtccttttttccctctcttttttattgaaggactaaagctttgaatgcttttgcagtttcttccaaagtttatcatctactcagc

SEQ ID NO: 153 - DNA, 401 bp, homo sapiens
attacttaaatttattaatacatttttaatatcagatggtaagttacaggatggagctgtgacacccttgtcatgaacaagcctacagttgtagacttatgctcacaaagggtgctcagctccctattaatgaattgattgatgaatcaattaaatgatgttgtgattattggctatcactgacacttgcagaaggtagcgctctcttgtgctgatttcatgttaatctcatctaactttaaggagctcatttcatgacacatggaatttaaaggccagtactctacaattcatggacactcttctaactcaaagtagctattaaggaaaccaatcttaaagtcaggatgacagggaccttaaagtaccaattccatttccagccttatagactcaaccaa

SEQ ID NO: 154 - DNA, 401 bp, homo sapiens
  variation 257: /replace=“t”
ccatacaatagatttcctactttaactacctaaatttgtttcaaaaccaaccactattaggggagttttgtgaacttacttcatgtgccattgaatttctaataggtgatatctaattttaaattgtagtttagagaaaaaatgttgctattctattgctgaaacataattgatatataaggagagtttggctttgaatacatttctttctctgtatatatttattttagatttcatgagcgagtatttgattcttaaaatattgaggacattgcaaaacatgattttactggatttttcagaatcgtaaagaagaataattcttttatagtttagtaactatctaaaataatttagacacttttgaccttattttcttgatgtttctttgagatgaggag

SEQ ID NO: 155 - DNA, 401 bp, homo sapiens
  variation 227: /replace=“t”
tgtcctttctacctttgcctggagacttagcagtgcctcccaccacagcagacagaaggtacatctcactctaccctctgacatcagctccctcatggaacttcctttggccaatgagccgagagcagaggcgtgagttgtgtctagccagagggttcaaatgtgcttgcgtgatttggcttggctttttgagcttcagcattctccatgggaaaatcatgccctgggtaggcattgctccttcaacctgtgtcctggaatgaagacacatggaacagtctggaacctaaaacaaagcctggagttcagctgagatcagctaaggtgtggctgggacctgtgcatatggaataaatgtttgctcttataggtaaatgcgatgtggaagttgttcattactc

SEQ ID NO: 156 - DNA, 401 bp, homo sapiens
  variation 4: /replace=“g”
aacattcgaggccagtttgccaaaaatcttggtgctaactgtatgtccaggttacagatccacacgcaaatgcagacccaacaacactgaggctcatcttttgggtttctccccatcttcttcaccatgactcagttctccaagaaaaatgttgttgctactttcaggaacagtccaggatgagagcttaaggtagagtccggacatgtgcatggaagcacagccatgattctatctaattggctcaaacaaaagtcttggacagtattttggcttttttttttttttttttttgagacatgatcttactctgtcacctagcctggagttcagtggtgcgatcataggtcactgcagccttgatctcccaggctaaagcaatcctcccacttcagccccat

SEQ ID NO: 157 - DNA, 401 bp, homo sapiens
tgcctagcctatattttttgtgattggttttctgagtctataaagaaaaaaggatgttcttcagctatttggggacttttggctccaccccaggcccataagtgaatgcaacaacaaggtttaatgactgttggacactgtgtttatcgagagacattctgcagggaagcagaaacaacataattgcctataaaaacatcctttaaaaagttagcagtctaatccataaaaatgaattttggcaggtgcaaatgtagccagggccaaggcagggcgtgcggtggatgggggcgggaaatccattcaggcgcggaggcttgcctgttgttgtccctggagtgcgccgtcctctctcttgttcctgcttcttttttactctcctttctcctcagcagccatcc

SEQ ID NO: 158 - DNA, 401 bp, homo sapiens
  variation 68: /replace=“t”
gcacaaccaggatttgaactaaagtttaccttcatctaaatacgcgtttttctttctctctctctctctttttttttttacccctatcgcatcatagccacatctgtggccacacaagaaatactttcccaacactcagtagctaattattcataaggttttagggtgacggtgggccatgtgcagagatgctttgaaagacttccaggaaaatcatgctttatggcttagaatttggggacatgactgtacctgaattgagatggacaggaaatcatttctccctccttcctgcttcagcacacccctcttcccactcaagagattctggagacgcttcaaggccatctccttgccagatgcctctgttttagaaaagtcatttcaagtgccccaaggag

SEQ ID NO: 159 - DNA, 401 bp, homo sapiens
  variation 54: /replace=“g”
gaggttcccgagggctctggggaggcggaagaggggacgcggtgtgttgagatacggggagaagttcagagcagcatgggcatgcaggtgcctgcagaggggcccatctgggctgcaccccagcaggacagggccatggcttggtgtgggttcggctggctgcacagcttcttgcaaagtttgtccccagggtccttgggagtagaggtctttcggaaggcaccatctccagagcgtgtttctgagtaactctgaggatgggttccgggttatcactgtgtgttggaaacagcagcgcccaggctgccggaggaggctggtctccctggcaggtcctggctgtgtctagagttttgtgggtcctggagataaggctccggtttgaggcccctggggctggc

SEQ ID NO: 160 - DNA, 401 bp, homo sapiens
gaagtgtctgtgtacacagctcctgacagttcctgcctgcctggctcctagcccagtgctgttagaaatctttcccaaacagctctacctttttgaaagtccagacccttgacatccatctctccctactgctcagaattgtctgtatctatcttttaccaatgggggaaaggaaagaaaaggcagataaagagtgctgcaccacagaaccttgggggattaaaaagaagcaggctgtgctgagagcctaggatcagaaagaatctcactgcaccctcaccttctttaacccaggcatagacttccctccatccattccaatacctggagtgtctcattggtgtagatgaaaacacggcagtcaggtataagcaggtcctggagctctgactagctcattt

SEQ ID NO: 161 - DNA, 401 bp, homo sapiens
caaatacatttgttttatgagtgtcaaaggctctaatatattaatattactaatatattagtacaaaatactaatataatactaatatactacaatgtattatgtctattatatataatacatcagtatattagttatattaatatattactaatgcattaataaagaaaaaagatgttaaactagacaggatccatcgtgtccttgtggcattttgatggaggccttcacctcaacagtgagttaagcctagagtatgaggatgagttctgtgcattagccactttaaagtttgtttgtcgttactcagtatacaacagagaaaacctcaaaatccctcttcacttctcttccaactctgcttttcccagtgtgagtagaatcaaaccatatgtactgtt

SEQ ID NO: 162 - DNA, 401 bp, homo sapiens
catgtaagaaggattttattgttcttctatgcttagcattccttctgctatgtggaatagtcctggagtaacttgacacacagaaagtaccatattttaaacagaagcaattcattgtagtggcttctgcgagcaatagaaacttgcagaacgtacaaggaattcaaacatctcaacagaaaaaagcccaccaagtttccacaaaaaactacaattagaactgccatatgatctggcaatcccatttctgggcatttatccaaagaaaaggaaatcagtatatcaaagagacacctccaaaccccatgttcattgcagcattattcacaatagctaagatatgaaatcaacctaggtgtccaacaacaaatgaatggataaagaaaatgtggtatatatac

SEQ ID NO: 163 - DNA, 401 bp, homo sapiens
  variation 136: /replace=“g”
actatgcattatatgtatcgaaacatcactatgtaccccatgaatatgtcaattaaaaaaatagtttcaccaagacccactaaatcaacagaattttgtggcaacattatgcagaccccactgttgagagtaaacactatttgatatgatatcaagttccatcagtttgcatcagatcatcagatgagagggccataagcgtcttctgttctcataattatgcagatttattgttttgaaacaaaatcaacttctctctaaaataagcaaggacactggggcaaccaactattctcagagaaggagtgtttttaatttttttcaggattcttaggaaaatcaaatatcaaataacatacaggaatgtagagttcaatgacaaaaaggaaatctcttgactg

SEQ ID NO: 164 - DNA, 401 bp, homo sapiens
  variation 61: /replace=“g”
cccactaaatcaacagaattttgtggcaacattatgcagaccccactgttgagagtaaacactatttgatatgatatcaagttccatcagtttgcatcagatcatcagatgagagggccataagcgtcttctgttctcataattatgcagatttattgttttgaaacaaaatcaacttctctctaaaataagcaaggacactggggcaaccaactattctcagagaaggagtgtttttaatttttttcaggattcttaggaaaatcaaatatcaaataacatacaggaatgtagagttcaatgacaaaaaggaaatctcttgactgtatgaatagtttcatgaagtcacagcattggtttaatagcaaaaattttatttggaaagaaacttacatttggat

SEQ ID NO: 165 - DNA, 401 bp, homo sapiens
tgttacctattatgattaatattgttttcctttttttcttgcaaatagttttaagtctccttcagttctattcattttttttttctctaaagagcttccccagcattcctccattttcttgaaactgtttatcatgcttgttttgtgtcaagccgtggggctctgtagagcatgaaacaagaataagattctattacttaaccttcatttaatagtatacatttgccaattaagagctttacacaaattttcttattaagtcttttcaacaacttctggatatggataatgttaattcttgtccatgatacaattggagaaattgaggcttaaagagatgaagtcacttttcaagtgctacatagatgctaagtgataaagaagaaattcaaacccaggac

SEQ ID NO: 166 - DNA, 401 bp, homo sapiens
  variation 45: /replace=“g”
cccttcttcattttaaatttattttatttttagttttgattaaaagacccacacataaacataaaatttactatctttaccatttttaaatgtacagttcagtagtgttacatatagtcacatcttgctgcaatgtccctccagaagatttctgtcatcttctttggaaaattttaagaaactattaccaaaaagtgtaccgagatatagttttaaggctgatgtaccaatgacaccacgtgaataaattctgtacattcacctgtaaagctcctcagcttttgggcttttttctccttattggctggttctaaataaattttatgaattcccaataaacggcttttgggaaatgccaggtcatcattgtaataaaaagtggtataaggggaaaacaactc

SEQ ID NO: 167 - DNA, 401 bp, homo sapiens
tgtatgacatcactgacagggactaatttaaaagatttaattattccccaattcacacatgtcttgatttatttttgataaagagtagcaaatgggtaccctaggattctatacaaataagccagtgtaatttgatggaataaacactgatttggatgttaagagattttcagctaaccctggctcttctgataactggcgttgttacttggggaagtcagttaatctctcgtttgtagaatgatggggctaaactaactggtctcttagaaattgtttcttcaagacctaaccttctgtggttctccttaatgcctggatgcaatgaccttactactgatggaaagattatcttgggaaatgttggccaaaactaaaccttctccccacacatctgttct

SEQ ID NO: 168 - DNA, 401 bp, homo sapiens
acagaaattagaagctaagcattgcaatttaaagaattataggaacttaggaatcataacaattttctttaaatatcaggttatttcccttaaataagttatgtgcaagcacttcgtaaattctaaggtcttaatcaaaattttgattttattactattattattgggctatgtgaaaaagagaagtgaaatactaagcccatgaaaaaataagattttatttaacataatttaagacacagagcacttgttcagactcaagtctgttacattcttacatagagtaactcacattattgttggaaccagagctacagttcgtatctttcttgagccttcctcagggtccagagttctgagagaatttcattatcaccatctcccaagaaaaacaagcttca

SEQ ID NO: 169 - DNA, 401 bp, homo sapiens
tattttatgtatttcccattgttctgctattacaaatagttgttcagtaagcattcttgtacatttatctttgtgcaattgaagaccaatatttctctcacttatgtagtgccacccttgctagtaggaatgttccgttaaatgcaaaacctgcagaacttggactcttcaacatgtttctactttgccaaaactgtctacgcctccctgtgcttgcttgctgctccttggctcttctttgtctttaatgttgcccttaactctaataaaagcctctgtcttagtttaggtttccccagaaacacatttaagacaaggatttgaatgcaagtggcttattcaagaggtggcctcaagatgcacaggtaggggagtggagggtggggcggaacagggaaggc

SEQ ID NO: 170 - DNA, 401 bp, homo sapiens
  variation 56: /replace=“c”
actaataacctggtctctcactcatcctgactgggctgccttcccttccctgtctgtcttcccctttcttctccctctcctcctgggatcacctcccatacaaactcctcaccctcatttccttgtcccaaggtctacttttcagggaacccaggatactttcctcctggtcctaagaaaacccaggcaaaccaagtttcctgctctcatacaatttacagcatgttgggttgaacaggcaacttaacaaacattaaaaacgaagtgtaacgagtgctgaggtcagggagcatagactggcggaaggcacagagccacaggggaccctaagctagcccaggagaggtcccagaagctgagctgggagtgactggggctccctgccttttcttccccttctt

SEQ ID NO: 171 - DNA, 401 bp, homo sapiens
ggttaaatgcctttcagggtagctgaaaactacttaccaatcagcagctggcagaaatgtgtaccaagagctctcatctggaagtgggcagctgactcatgttggagttgtcatccctactccagcagccttctgggtgaaaagttcttcaaagcacatgcatgtgaggccagaccatttctcttctgttttctatgctcctcaccaaaaaagaatgagtcatggagctctctaattcaggatccagtagagcatcagacagaatgcaggctgttgtttgaacatccgtctccagatagactgcagtgtcatttaaacaaagatgtatgcatttgaatatggtgccatacaattcttgtgccgaactggcatatcttctgagggaagattttatagaaata

SEQ ID NO: 172 - DNA, 401 bp, homo sapiens
  variation 27: /replace=“g”
ggaaacaaaaaggattataagaaagtaatataagcaactgtacaccaacaaattggataactgaaatgaaatggacaatttctagaaatacacagtgtatcaaaactgaacaacaaagaaaataaaaaatttgaatagacctataactagtaagaaaatctcctgatatagagaagccctgggctccatggctttactggctaattctaccaaacattaaaatacaaattaatgccaatcatcccaaaaattttccaaaaaattgaagaggaatgaatgcttcttaacttattctataaagccattaccttaatactaaagccagacaaagacattacaaaaaagaccaatattccttataaacattgatgtgaaaactctcaagaaaatactagcaaatt

SEQ ID NO: 173 - DNA, 401 bp, homo sapiens
aaagaaagatatttctagcacatatgagtttatggatggagactcatgcctgccacactgtgaaaacattcttcccttcaaaatccctatcagtgtaatttgaagtgaatattctagtaccacatgcttttggactctaaggaagagatataatgtacgaaaatgtggtatctgaatgcagcaaattgaaaaataccagtccagatactttggggccatgcagagagtttcccagtcaggtgtaagtgtgggtggtggtggtttgaatactgaagggatggacaggtgggggtgacaactaagagctgccacctggggagaatcctaggcagagtctgggcctggacaacccaaccagtagtgaccgtgggccggacgagcctctgggctctgcagctccg

SEQ ID NO: 174 - DNA, 401 bp, homo sapiens
  variation 4: /replace=“g”
tacactaattcatgcaattgtatattttggaggtaagtgtaatggcataggtttgctgtaatggaaccgattggaaaatgatctcttggagaaaatttattgctggtcataagatttctttggtttagaaaaatgctgggtttagtaaaatctgaggcactaaatgaaagtctgctttaataaaaatgtggatctgagaactgtggtttttagtaagtgggcatttgatccattaaaattgtattggttttattctttacgttggcttttcctgaaatccatcagtttgaagcatgaagtaagcacttgccagatattgctccaataatgaaacattgaaagcaaggccctgaataactgttaaaacaagtcctattaatcagctgtgacttgtccctttg

SEQ ID NO: 175 - DNA, 401 bp, homo sapiens
  variation 106: /replace=“c”
atccaatcaaccatcacagaaagaaagcatgaagtccagatgactggaccaaaagaacaccctaggttggatggatgctggatggccaactggatgggaaaaaagagggtcttgataacattctaattagccccaaaccctctattcccatgagccagtctcttctctttaagttgatgctagctgccgttttgtgttatctgttacagactaatacaatttgcaataattgaagatgcaatatttattgatatattattatcaattgaagtccatagcttacattaggattcactcttagttttgtatgttctgtggattttgacatatgtaaaatgacttgtgcccaccattacagtatcacatagaatggtttcactgccccaaaataccctctgttc

SEQ ID NO: 176 - DNA, 401 bp, homo sapiens
aaatggacttaagccattacctttggctgtcctagcaggggagggtttcagactttcatttctttcccagcctatggtgtgaggggaaaagttgtcttggcatggagatacactgtcagtgcagactcctcaaagatgaatggatgtgcatttaagaacagatagctgagaacatctctgaacatcagtttctgcatataaaaaatagagccaataaactcttcctcaccagtttcttgagaaaattaagtgatataacatgtgggaaatgcctgccttaggagacatctagaccaacactgttgcaatgatggacgtcttctatgttctgtactgtccaacagaatagccaccagccacatgtggccactgaacacttgaaacatgattaatgctatgaa

SEQ ID NO: 177 - DNA, 401 bp, homo sapiens
  variation 280: /replace=“t”
attaagtgatataacatgtgggaaatgcctgccttaggagacatctagaccaacactgttgcaatgatggacgtcttctatgttctgtactgtccaacagaatagccaccagccacatgtggccactgaacacttgaaacatgattaatgctatgaaggaaattaatttttacttttgtatgactttagttaatttaagtatgagagccacatgtgcctggtggctaccatgttggatggctcaggtctagacaatatgtgtaatgttgatcatgatgacgatcatattagttctgcttcctcaagttggagtcaaagttttatgctcagggaacttgtgcctctaggatgggatcagaaaacaaaacttttccccggcaagttgtatcacagttaggttt

SEQ ID NO: 178 - DNA, 401 bp, homo sapiens
  variation 168: /replace=“t”
ccatgttgttgcaaatggcaggatttctttcatttgaaggctaacattccattggatgtatatgccacattttctttatccattcatctgttgataggcatttagattatttccatatcttgccttttgtgaacaatgctgcaatgaccgtggaagtactggtattcctttgagatctgatcccaatttcatttccactggatatatacccagaagtgagattcctggattgcaaggtagttctagttctataatggctacaaccaatttacattcccaccaacactgtgtggtttccttttctccacatcatcaccagaatgttttgttttattttatttttaaaataatagccaaaccatttggatatggtttaaatattcccaatactagatcaacta

SEQ ID NO: 179 - DNA, 401 bp, homo sapiens
  variation 390: /replace=“t”
aacttcaatgaggaatgcaacagagagagagaaaaaaagaaagatcgggtgattgagtagacattgaggtctgaaaaaatgtagaagtcaagtcgaacaaataagcataaacatgtacttttaggaagaagtccatgttgctggtgccctgctcacattgctttggcccagccttgaaggacagctgcagagtggattctctcatctcccttcccagaggcatcctctggccaaataagggggcattctcggccatcaagctgagcaggccagtgaggggtgggcactgaggggcacacaccagcttgtgtcccactcagaggggcggttctgaggcatgctccctacagtctcccagagtggccccaggagggctaagcctcagttgcccatgatggcaa

SEQ ID NO: 180 - DNA, 401 bp, homo sapiens
  variation 12: /replace=“t”
gagtggattctctcatctcccttcccagaggcatcctctggccaaataagggggcattctcggccatcaagctgagcaggccagtgaggggtgggcactgaggggcacacaccagcttgtgtcccactcagaggggcggttctgaggcatgctccctacagtctcccagagtggccccaggagggctaagcctcagttgcccatgatggcaatgctcattattgcaccctcgtggcttccttctctttcctgcctcatttcgtactcccaagctgtgctcccctgcatcacttcctaaactaaatcccttcatctaagaacttatcccagggtctgctttggggaagaacaaaagacaaagcacttatgatatactcaagactaacagaagcataagcttc

SEQ ID NO: 181 - DNA, 401 bp, homo sapiens
  variation 6: /replace=“g”
gttttataaaatttatgatgtgaggttacaatatgcctttttcttgtgattcatccttcttgcaagcttctatgtagcaagccggcaaaaggcagtggaagggacttctggggacggtcctgtcatttcctgcagtattttcgtgagtcattcagcatcggtctcttccctactcctattgtctggctggagagaagcaccctttcataggtgagcttttcctacaaacatgagagattaaaaataagatcttcaaatgaagaaataatttgacatttattcattgaaaatcactcatcttcaactcatcttccatttacagctctacccctttcttttatggatggagaaaatgaggcccagaaaaataaatggatttgtccaagataaccctgattgtt

SEQ ID NO: 182 - DNA, 401 bp, homo sapiens
  variation 213: /replace=“g”
tttaaggtatggatacatctcaacttgcttattcattcacctgttggtgggcatgtgagttgtttaagttttgggttattacaaataaagctgcttgaatgtttatgtatatgactttgtgtgtatttatgctttcattttttctttggtaaaaaccaagaatggaatagctgcatcatatggtgattgtgtttttttttaaactttataagaaactgacacattgttttccaaaggggctgtatcattttacattctcaccagcagtgaatgagattctagttcttccatatcctcaccaatacttgctgtggtcagtctttttagctattctaacagatatctcattatagttttagtttgcttattcttaatgactaatgatgtcaagcatctattca

SEQ ID NO: 183 - DNA, 401 bp, homo sapiens
  variation 63: /replace=“c”
gttggtgaatttttgtcttagcatctgcttccggcagaatccaaacaaagacatgagaattaaatttttggtttcattcttgttttattttcttaacatacatgcttatatgtaaaccaattttgaattttatgtatcaacaatttaagttctactgctttaagattatatggacattaaatgtacagaaaaatagaaaccaacaaaagataaataaattaaatacatttttaaaaaagtctcgcgagttgtggtggcttaatattgccgtgagctaatgccttgagggctactctctctggagtgggaatggagctctcagtaattagacagttttggccagtggcttgtcacacggattgtgcattcacctggctttaggaggaagaggaggcatggca

SEQ ID NO: 184 - DNA, 401 bp, homo sapiens
  variation 172: /replace=“g”
taatgtccttaagatttaagcctcagttctttgcgctttctcttacttgcagcttaaagcattcctaactgatgtttaagaagtttggaaaagaagactagaagagaaaaacttactcattgtccttcttcttttttttgccctatacccaaaataaaaattttcctttctatgtacaaatttgcacatagctatgaccaaatggtgtatgtacaactgtctttgttgtttttaaatctatcttttatcttaaaattaaatcatgtgttcattgcagaaaaactgaagaatatagaaaatggtaagggagaaaataaaatcactcagatacctaccaagagagaacaaatgttaacatactaaggtttctttttgtaatttttcataaatttgatgtcctt

SEQ ID NO: 185 - DNA, 401 bp, homo sapiens
  variation 142: /replace=“t”
ccaccagtttttcctgaggacaaggagcctcttaaatctgaactatggtgaagtgttttagtttgagatcccgggaaatagactctgagacaaagaactgtgagtaggaggtttcatggagagtgctctcaggatccacacctgcgaaataactataataataataataataataataataataataataatagtgaaggcctggactgggtagaggaagaaactgatccaaaacaaacgtgcaatgaggcctcagccaattcctggggggcagatctggctgtgtacagcagttcctgaataatgtcggttcattcgatgttctttccttataacgttgatgagaaaacaattgatttgcacctggggccaccatctgtgtatatttgcacaacctcccc

SEQ ID NO: 186 - DNA, 401 bp, homo sapiens
  variation 48: /replace=“t”
gactgggtagaggaagaaactgatccaaaacaaacgtgcaatgaggcctcagccaattcctggggggcagatctggctgtgtacagcagttcctgaataatgtcggttcattcgatgttctttccttataacgttgatgagaaaacaattgatttgcacctggggccaccatctgtgtatatttgcacaacctccccatagctgcatgggttttctctgagtactctagtttgctctctcattccaaagtcatgcacattaggttaattggcatgtctacatggactcagtgtgagtgaatgtgagcatgggtgtgaatgtgctctgccatgagagactgtcctgttcagggctggttcccacctcactccgtgagctgctgggatgggctctggccaccc

SEQ ID NO: 187 - DNA, 401 bp, homo sapiens
  variation 357: /replace=“c”
tagcctatggaaaaattgtttttgttatatgtcattttgcttaaagctgcagtttccaggactctatcaacaacattaagtgaggactttgtgtgatttgcggggccaagtgcaaaatgaaattgtagggtaccttgttcaaaatgcaagaaaaaagtgtcattagaggtacagaaacacactttttttttctatctgtggtgtctcaaattgttatggtggtgttattttcttctaaatgtctttttaaggaaagaaaaataaaaatttaaattattagcaacgatttttgccattcatctttatattatgcattgctaattttaaacacaagtgtgtttaattaacttgtatggagactcaccaaaatatacattttgtattttattactcatgcgtgt

SEQ ID NO: 188 - DNA, 401 bp, homo sapiens
cagcctcccgaataactgggattacaggcatgcgccaccatgcccggctaatttagtatttttagtagacatggggttttgccatgttggtcaggctggtcttgaactcctgacctcaggtgatccgcctgccttagcctcccaaagtgctgggattacaggcgtgagtcaccatgcctggccgatcataacagtcttaactgcaatttgttaaataataatttaaactgaaatgatatttacctgtagagaccccaggatccacagttatatctgtttcccaacatcagagactttcttttatggggcttgcaggaattctaccaatatttactgagtacttctgatgaatcagacattggtctaagaactgtaagagactgaaagatcaataagaaaaa

SEQ ID NO: 189 - DNA, 401 bp, homo sapiens
ttttgagacaaagtcttgctctgtcacttaggctggagtgcaatggcgagatctccgctcggtgcaacctctgcctcctgggttcaagcaattctcctgcctcagcctccctagtagctgggattacaggtgcccaccaccactcctagctaattttttgtattttagtagagatggggtttcaccatgttggccttgaacgcctgatctcaagtgatcggccccccttggcctcccaaagtgctgggattacagacatgagccaccatgcctggcctgcatgactgactcttattgtttgtgactataaacattacttccccagaaacttgcttcctaacctacaacttaaaatagtgtttggtcactatttgaagagtagacaatcacactttaaatag

SEQ ID NO: 190 - DNA, 401 bp, homo sapiens
  variation 392: /replace=“t”
gggattacagacatgagccaccatgcctggcctgcatgactgactcttattgtttgtgactataaacattacttccccagaaacttgcttcctaacctacaacttaaaatagtgtttggtcactatttgaagagtagacaatcacactttaaatagataatctaatgctatgccctattgtattcccctcataacagttactagtgtcttgatattttatttctatttgtttattttttttgtttgtttcccatctgaggagaataaaagtctggtgaaagcagagaccatatttattttattcatcattctgagaattggatgggacaatcagagctcagtaaatatttgtaatgaatctgtaaaccaaggacaacggtaggatccctctcacaggactt

SEQ ID NO: 191 - DNA, 401 bp, homo sapiens
  variation 10: /replace=“t”
taacagttactagtgtcttgatattttatttctatttgtttattttttttgtttgtttcccatctgaggagaataaaagtctggtgaaagcagagaccatatttattttattcatcattctgagaattggatgggacaatcagagctcagtaaatatttgtaatgaatctgtaaaccaaggacaacggtaggatccctctcacaggactttatgagtgttaaccaagataatgcatgtaaacaccttcgtacagtgcctggaacacagtcaggtctcagtggatgccaaatcattaggttcaggtaataaaattgtatatggagctccattcttgccctcaagtgcctcaaaggccagagtgaagaaggagatggctcacaaatgaatatgtcatttacta

SEQ ID NO: 192 - DNA, 401 bp, homo sapiens
gaataattggtgtgtctggactcagtaacaccagtgtcagcatatgccactctggtgcccaaggacagttcattgatgacaccattggtgcttgaggactggcctatgtggcatcccagttccccacaaaacttcaccacaacctccactaacaactgcaccctaagctgccaagaaattatagacactgctgatgctgtgtccagctgaagaagccacacacaaactacactactgcacatgtccagaatcaaagccaagatgccgtacacaaccaacgttatagctacatctccaggaaaatgttttcctttacaaaagcaactggaaaaaattggaagaagtgaccattacaccaggtgcaaagatatcagtgtaaggatagaggatacatgaaaaag

SEQ ID NO: 193 - DNA, 401 bp, homo sapiens
  variation 48: /replace=“t”
agtatagatacacaatgaaatactattcagttatttaaaaaaaagaacgaaatcaagtcatttgcagcaacaggaatggaactagaggtcattatgttaagtgaaataagctggacatgaaaagacagataccatacattccactcatctgtgggagctaaaaatgttgatctcatggaggtagagagtagaatgatggacaccagatgctggaaagggggtgtgagtgggaggtggtgatgaagaggggttggtcaataggtgaaaacatgcaattatctagaaggaataagttccaatgttcaatagcaaagcagggtgactatggttatcgacaatgtgttatatacttcaaagtagccagaagacagaacttgaaatgttctcaacacatagaaatg

SEQ ID NO: 194 - DNA, 401 bp, homo sapiens
  variation 122: /replace=“g”
ttctattttttaagagtccagatttttatatcatttgttttttatagaaaatgatgctaacttggtaaacttgctccagtcactgttcttcaacaggatcctaaaattaatttcgattttgagagattttcctctgctaaggccaagcgaatgaattttcaacacaattctttaagtgaaatttagaagagaaatgaagggtgaaatttgcatgtcatgttcaaatgttgtaattctgtcacacctgatatgctattatcatagaaattataaaaatattttaactgtattatatagattgttatgacgcatacaaattatcacttcttttacaataatatcaggatccatgcagggaagatatatagttcctggattcttctttcaaatttttctgtgct

SEQ ID NO: 195 - DNA, 401 bp, homo sapiens
  variation 15: /replace=“g”
taatttcgattttgagagattttcctctgctaaggccaagcgaatgaattttcaacacaattctttaagtgaaatttagaagagaaatgaagggtgaaatttgcatgtcatgttcaaatgttgtaattctgtcacacctgatatgctattatcatagaaattataaaaatattttaactgtattatatagattgttatgacgcatacaaattatcacttcttttacaataatatcaggatccatgcagggaagatatatagttcctggattcttctttcaaatttttctgtgctaggatattatcaagaaaacacaaaatatcatgtcttgtttaatttatttaaatctctcttcacacagcactataatatccataaaatactctctgcatttattttgg

SEQ ID NO: 196 - DNA, 401 bp, homo sapiens
atgttttggatccatttttactcccacatttggcaaataatttattcatgaacaatgcaaatgaaaattttttctcagttgagctgttgtgaaattattacacaaaacattttgaaggttatcaattgtaatgtgagagccaaaaccacgtgtgtgcaggcccatctttctggttggggattatactatttgatataaatcacctcaagaaatagccgtagatgagagaaataaacattttaattgttcttataatgaatgataagtgaagggaactataacctcaattgcaaatgttaacatttacaagatgacctgtttataaatgtagctctgccttatacatgtgctcacatccattttgaagtatccaaccagagcgctgatagattagtagtttt

SEQ ID NO: 197 - DNA, 401 bp, homo sapiens
taaagaatttgcaaacaagggaccaatcatggtagtgagtaacttctttgtctcttgctccttttgtacaaaatatctgatttttctcctgcacttgtctgtatctgactcactggttgtcttcttctttagtttaaggcctctggaagcccagagataggaggcagggacgagtggagggcagttttctctcgactcttcctgcagttatttaacttctgtttctaatttataaggctttcttggcatatgtaagcctatgcagtcaaactgagttgtggtccaggagaggtcatggaccagtgtcagtcatgtgccacatcacagagtgacgtgagattcggagatgagaaaaatgtaaaaagaccctacttttttttcttggcctatcccaataaaat

SEQ ID NO: 198 - DNA, 401 bp, homo sapiens
aaccaggaccacactgtaacactgccacctctgcaacatacatattagtgcaaattggggtaaagggtggggctgatagaatgtagagtgtaatttaggaaccagcctatggtgagatcttgcaactgctggcaaagtccgagccactatcctttgagcaatccattcacaatggaaccagaaaaacaatgtctatttagccagaaaaaatttgaatggacaagagtgatgtctctttaccaatacaagagagataagattactaggaatagaagagttacatggtttctctgactgccttcacaaagttcagggtggctggaggtctctgaagaaaagcctggacatcagtgatgaatcaatctctcaaagcctcctgtagcaaacattgccaagggcct

SEQ ID NO: 199 - DNA, 401 bp, homo sapiens
  variation 307: /replace=“t”
tttttttaccaaagatcgaaactctcaaaacattttcagaggcttcataaaatcgttatttgtgttggaattttatataccacccaaagctggatgaaatcaaagagccaatggctaggcttggaggtcattttaaaaactgcattttttcaccattataatatttattgcaatgggctaggccctacgcatcagattggcgatattattaaggcatcagaaggccaagaaaatctgaaaataaaatggtttcccaggtaaagccatggtaagggtaggcaaaactggcatcaggccaaccgtttcaaaccatgcatcaaacttttgaggtcaggcaaaattgaaaacacatttggagaagttaattcaccttaattttgaaacccatgtgtatttcaagg

SEQ ID NO: 200 - DNA, 401 bp, homo sapiens
ctaagttatctagtttaccagcctggagttatctgtagtatgtgatagtatagtatagcatacattttcccccttttagtatctacatggtttgtaatgatttcccctctttcattcttggggtaatctgtcttcctctactctcctgacacatactttgggcagatcccacctctaggtacagaatgtgattggcaattcctgtggaccaatctgagcattgctttcacttggctgtcctgattgctttgaatggagagagtaaggcgcatcaggcagatacaaagcattttgctgagattactggcactatataaaccctgcactcccagggagcagtagaaatcttgagaatggatccattagagtgtaaggcagacgttaaagatggagaaaccaac

SEQ ID NO: 201 - DNA, 401 bp, homo sapiens
  variation 35: /replace=“t”
atctccacccagaaatggagtgctgtagtaaggcccactcaagataacgatggcaatgggaagctggagtgacagatggcacacgatggcaaacagcccatacacgttagtgctttctgtggaaaataattttgtatatatataaatatcttttataatttaggaatttccacattattaagtcaggatagccagtatagcagagatacaggtgtccaatatccctttcatccttcttcctttagtaatagtttatctgagtgtatactgagtgtggtcccttgtccaaaggaacctttaaagtggtggtggtcacatgctaaacttatagtcagaggtatgtgaatggcgatggtggatatcacctctcagtcatgctcatatctttccttctgtccctt

SEQ ID NO: 202 - DNA, 401 bp, homo sapiens
  variation 37: /replace=“g”
aagtaaacagaaaatgaataagctgtggactttgaaagatagtttttgagttagaaatctgaagggggagaaaaggccacagaaacagggtaaacagttaaagcaaacattttagaccaaaatagccagtagacagcagctgagaatggacctcactgctggtaaagaggcccaagtggattaaattgatcaaaacagtgcttgggagatagcaaggaattttatttttcattatttctcttcaaaaaggcatgatggaagattagacttctgtaagaacaaacgtttgaaacttttctattgctactgtttctgtgctttgcatttgatgttggttatgaaacgtgtttttctcagtatagatatatatgtatatttttatacatacacatacatatata

SEQ ID NO: 203 - DNA, 401 bp, homo sapiens
  variation 40: /replace=“g”
agtttagtatgttatttagactaatattttataaatcatattctggaattaagctgagtaaggccaggaagaaagagggttgaggatggatgtaatgtaggaaccatgcctaggaagcgaggaaaagcatcataaaaggacagcaacaatgtagaggagagaagttaagcagtcagtgagaggaaggcctctgaacccacccaggtacttagctgtagatggacatcagttaattcgatgacccaccaccccatctcaccaataaaaaccaaactctttgggaactgggaccagtactgaactagtgggtcaaaataaagtaaaaaagaagcccacttcaagaaatactcctctaaataacttcagagaaaaggaaattttgagaaacaagcaatcacaca

SEQ ID NO: 204 - DNA, 401 bp, homo sapiens
  variation 55: /replace=“g”
ggctatgataaccaaaacaacatagtactggtacaagaacagacacataaaccaatgtaacagactagagaactctagaaacaagaccacttgcctacaaccatctgatcttcaataaacctgacaaaaacaagcaatggggaaaggatttcctatttaataaatgttgttgggagaactggctggccacatgaagacaaatgaaactggactccttccttacaccatgtacaaaagttaacccaagatggattaaagacttaaatgtaaaacaccgaactataaaaaaaaaaaaaaaccctaaaagaaaatctaggcactaccattcaggacacaggcacaggcaaggatttcatgatgaaaatgccaaaagcaattgcaacaaaagcaaaaattgacaa

SEQ ID NO: 205 - DNA, 401 bp, homo sapiens
  variation 167: /replace=“t”
atatgggtagcttcctagaaacatacaacctaccaagactgaatcattaagaaatagaaaatcttaacagacccataatgagtaagaagattgaattagtaattaaaaatctcccaaaaagaaaatttccagacattatgacctcatggttgaattctactgaacaagtaaagagctaaaatcaatcctataaactcttacaaaaattgaacaagagggaatacttccaaactcattttactaggctattattaccctgataccaaagcaagacacaaacactataaaaaaagaaaactacaggccaataaccctgatgaacatagataaaaaaatcctcaagaaaatgacacaaactaaatttaacagcacaataacaaagatttatgaaccattataga

SEQ ID NO: 206 - DNA, 401 bp, homo sapiens
  variation 71: /replace=“c”
gcagaccccactgagggtataacacacaccaggtgacttcactaaaatccccacggtgaaacacatttgcggcccttttccctgcccctggattttgctaaggatctggggggttctgtggaagtctgaaggtcagtggacaggacgagtttggagatgcgcatttggaaactgtacccctcaagtgacaaggaaatgagcgtggatttaagaagataatgaccgttctgtgaagttttaatggaatttttaagcaagaaaacccttaccctgtgaaaacttcagtctgcacagaagaattggtgggagaaggaagactttgcctaagatcactttatatagcacaacccagaccggggagaaggcagagttatgcccagcatatagccttggcagcatat

SEQ ID NO: 207 - DNA, 401 bp, homo sapiens
  variation 134: /replace=“t”
gaaagcagctttctcccaaaagcagacagaggatgggataatgacagtgaagtgagaattgaagagttaatagtgtccatgttatgctctttaaagcagacctgttctgttgtgtattttggcacatggttttcagacatattttagtcaatctttgcaaagttgaaagttgattcagagccagatgttcagaaatagacagagaaggctattgtaatttttcatccctcagtttttgtacagaaacttcattgtaaaaacccttctagactgatggctgcctcattgaaaactgggtcaaaaatatggctctgttagaaaatctcttggattacaaaacaatgcctaaacaaaaacaaatagaaatggaaaccgccctacagatgtgagatttgtgccaa

SEQ ID NO: 208 - DNA, 401 bp, homo sapiens
  variation 360: /replace=“g”
tatacgcatatctataaatatttctatatggaagcagtgtctataggaagctacacatgagttcatactgcgatcatcaactttaatccattaccacatgaatcattctaacctgctccccttgcctatctctaaattcttactccaacagaaaaaaacaggctctcaacatctgccatctacttacttaattattcaaacctagtgcacatgtattgcagtactagaattgttaacccatacccacatgggaaaaagtttatcaactagagtacagtgcttatgtacaatgttttttgcaatctgaggctccactgatttctaaaattacttaggtcagcatcttttttcttcaccctattcagtgaagtgtttcatccgtttgtaatacagttagatta

SEQ ID NO: 209 - DNA, 401 bp, homo sapiens
tgtatttgggactctgagttacagcagcctttttcctactccaccctctacttatttggtcagttgtttctcacaggctttgtacagcgaattcaccctagtaagtacagcaggctgatacttaattttactctgaatgctggtagatgaaaacaacagtgtcattgcctagtaaatggaactaattctcagacttagtgccaaggagccagaaagggcagtgatttcatgacctagttaaaggcacagtcattactcagctcccactaattttggtcatgtaaaaacacaggcccaatgatactagatattatactttctgcagataaccacggcaacatttcccattcatatgcttttctagaaccttgcctctcaccattaagaaaggaagcgtgtgg

SEQ ID NO: 210 - DNA, 401 bp, homo sapiens
ctattattgtccccattttacaaatgtggacactgaggcacagaaaaaataagtaattttcctggtgtcatgcagacagtaaatgtggaagctagggagcccggctgccactgcttctcagcacagcaccgggctgcctttgggataattgtcatattccctttcttggcctcaagggcttccatatccagtcttccccacgaacccccaacagcttcaacacatgtcaaccacccccagtttaaagcaagttgcccagaggaattttttgcgacttttgagtcttttcatttaagaatatacttttatgctcaaacataaaatctggtaataaggttattattatatctatatctcagcttcatccaatttgctgtcgtctctctttattatatacttga

SEQ ID NO: 211 - DNA, 401 bp, homo sapiens
tcttgtttccagagataaattagtaggaaggggtgaaaaggagaaatatttgtgaatcatacagtagtcacagctattggttgatgtctaggatattaaaatgtgaaagtattacgactaaaaatcctctgctgtctaatctctcacagcttatatttgatgttccctaaaatcacggtgtccaaagacagaccatgtttctctagaattcctgtaatgaagatctttgcaagatgccgcaattttgaatgtcccaaaccacaaagaggtttcagttcttcctttcaatccccaaccttgaactgaagcctctgcagaaagctcccagctcactagtgtggacaggcaactacttgtgcttctatgagagagaaaaagtgagcagtttccaagagacagaa

SEQ ID NO: 212 - DNA, 401 bp, homo sapiens
  variation 248: /replace=“t”
ctaacatgtattatttcatttttctaataagaaaaaataaacaatgtgccaatgtaaatacaaatcacaaaatagttcacctttctggctgcctgctagcagatttttgttttattttactgcatgtgtggacaagccatttcatgtacttttatctctggtttaagttatacagaagccattctgtttccctttgtaagagtttcatttaaatgtccaggtgtatgtgggtgtggtggaattaggaggaagagcgcttcgcccccatcaattctgcagttgccgtggcagcttagccgtgagtgggtcaggtggccaaagcagaggccgacccccgccagccgttgctgtcaggccccctctcgctctctggttcaggagaacgtctctagaagagcgtc

SEQ ID NO: 213 - DNA, 401 bp, homo sapiens
  variation 11: /replace=“c”
tttgcttcataacaactgtcttggaataatttttatttacctaaaaataaggtaagagtatgtcaaagtttaggcatagtgtttattaaatgcatagtttgtatcaatgcttgtaagctgtaattaggaatgatatgtggcattatattttacttttgttaatgttcactgttaacttcctgaaatgtaagctgtgctcacgtgaaaaaaaaaaatggatcagtttttctgagaagacactgtaacacaaaaagacctacaggatagtatttgaatagttaattttaatgcaaacaaaaacttctatttcagcattaagaacacatattcttgatataagaaatagaaaatattctagcacatacctgacactggtaaaatatatgtattctttttcccta

SEQ ID NO: 214 - DNA, 401 bp, homo sapiens
ctctcaggtttgaaactgtgtgtgtgactgtgggagtaaatagtctagtttcccactcttcatgtgtaaatagacacaaacaggtgacttttcagagcctgggacagagaagacgaccccccagtggagacgactgaacaggcccctcatgcatacagcatgccaccagtattgcccagaaatactgtttgtagaaaatcggaaaggtagccgggcccggcattaatgctagggtgtctgagatgagtcgaaggaacagctattctttttccctttgttatgtttcttccatttccaagtggatgaagctctaactcaacctttcatttgtttcaagagggaggtatcagaaccacaacaaatggaatgaaacctaccactaccacctgcctggcacaaag

SEQ ID NO: 215 - DNA, 401 bp, homo sapiens
  variation 388: /replace=“g”
gaaataaaaaagaaaaagatctttcttttctcctggcctttaacttggcaggctttatctgctcttcagagtcactgagatgagaaccaggtggcaaactctgctttccttctcctccaggcaacgcagaggcattcttaactcttaacctgcagactttgaaactaggcacagaactccagaggggataataacaccagcaaatgtttagctaaaaaatttagccatacatgccacacattgtgctacacattgagctctttggtcctcacatcaacgctgaattaggagctatgattatccccattctttagattagtaagctgggtatcaccaaggagaagtacttgccccaaagtcacatggctcatatgtgttggaagcaagatttgtgacagatc

SEQ ID NO: 216 - DNA, 401 bp, homo sapiens
tttttatttgtgaaacaatcctaggtctttgtgttccttttacctgattcccaaactgaacaaacccaagtaccaaccattccccttggatttttagtcaactgaaggttcagagagctgctcccttggggaagaggctgtggacatgacatttgcacatgcagcatatcaacttggaataatttccccctcatgtgggggctactcccaaggagtttattctaaacccaaagtacattagttgcctgtggcaccttagaaaaataaactatcctggggtccctggttttggaatgtcatttcctttatgatatgtgtctttgaaaggcaaggagtggcagtaagcagagtgatataaacatggggcctgggctttaattctgtctgccctcatcctttat

SEQ ID NO: 217 - DNA, 401 bp, homo sapiens
  variation 138: /replace=“g”
ctctttccttctttctttctggcttgctttccctcctttgctgctggtgaccacccatgcttgagcagtgttttggctgctttcccactgatgagttttccaaaccttgtcaggagctggcagttaaatgttctgccaccagcttacagaaaatacaagttggattgccttgttcctgctttctttcaaacttgcatgttaatccatcttcaccttgattcctagaagagtcaaacatctgatcctcaggtcaagcctgaaggaaatggttgtcccagctgaaaataagatcgatcccaatttgctttacagtactcttgttaaagatcagtgtgtaatattttctctgtgtgatgcaatgcttcttaataaaaacaaaaagggatgtttgaccttaactt

SEQ ID NO: 218 - DNA, 401 bp, homo sapiens
  variation 226: /replace=“g”
tccatgtcaacagcaggcggcttatattccagaaagttaagtctgtgccattctctgcctgagccagtatgttacgaagagcattaccatggcgatttcacattctgaaactgtgcttgtctcttaaatgccacgtgccaatgagtcatagaggtgttttgttctagagccatgtaccccccaaaaagggagtttgtacagatttaccaagaaactattattattatctacagttcatgcagttacaaaactatctttgcctccttttactcacaatttgaacccaatcttttctaaggtgctggcctcccaatattgctgtagataattcagccttggtttcttatgattaaagattatgactcacaccagcctacaagcctggagaattatactgaagt

SEQ ID NO: 219 - DNA, 401 bp, homo sapiens
  variation 229: /replace=“t”
agaattgctggatgcttttaagtcatcctccaacatgaaccagcttcttataaaagatgagacagttgcatttatatatccaaagctagctgctatattttctaagtcatgtctcaagtagagcatctagaaaaaacctgtatcagtagtgcttaagtctggcactgcagggaagctggtttaaaattacagagcctctggaattttcaaatgttcaaaacatccaacctagagcctgggaatctgtatttttaatgagcatcgctcatgcattgctatggaacacacattgcatatatagcattttctaacatataggatcaggccagaggaagaatgtctgtgtatgagaactccctaaatgatgcacagttccactgtctggttacagttgaaaccgt

SEQ ID NO: 220 - DNA, 401 bp, homo sapiens
tgaggccccattggtttaaatatctcatttcttacttgtcaaagtgcatgaatgtccagcattagaaatgcagttccttggggtctggagggcattgagggctcccagccctgcttcttaagtgcatctgctgctgcatagtatcaggcttaagtcttagcatccacaccttgtcagcaattaggtgacagctaactgtggtcaaggctttctggggactgaatgaagaactggggtggcacaggtagtgaaggagggtgagaggctggtggggccatgacagggggtgttgggggttggcagagcccaaacttccctgctttccctggaactggctgcaccaagaagttgaggctcctggtttgtttggaatcaggtatggtacaatccattgtcccgtg

SEQ ID NO: 221 - DNA, 401 bp, homo sapiens
  variation 267: /replace=“t”
gcctcttctggacgtgagacaatttgagtgcagtatttctctagttgctgacccttcccagctcctcctttcaggggatgtggtgtcatcctggcattgtcctgtcctaacctagccctgctggtctccatggctgcagctggatatccaaacacccaacacacataatcatggctctgtgccgtgaggaggagacaagaaccccccaaaccattctgtcgagtttcctatggttgggttctggttgtatttctcttcttttctttcctttccttttcttttttttttttctttgagacggagttttgctctttgttgcccaggctggagtgcagtggcgcgatctctgctcattgctacaacctctgcctctcggattcaagtgattctcttgcctcagc

SEQ ID NO: 222 - DNA, 401 bp, homo sapiens
  variation 53: /replace=“g”
cacatgtgagagaacagctttccagaaaccacacttgagtccacagcagagcagaaggggcaggaggcatcaggaccatttccagatttctctctggccctgattgtctagatttgcatccaatcactgcaagagtgtatacaaaagttgttagaaagatttgggtttcacacagagttatgaaaacaaggtagaagcctgatgtcttcatttcccatgaaatcttgtgggtgtaaatgaaacatgttttagtctcaaatcctggagctgtggggttcacaatggagaaatatccaggtctcttgaacaagactggacgataaccagggacagttccctttttcccccacattcatttatgagttggtcaaactgttgtctattcacaggagattcttaga

SEQ ID NO: 223 - DNA, 401 bp, homo sapiens
  variation 329: /replace=“t”
actcttcttgagcagtgaacatcaagagatcatggattaaatgggaaaattagtgtcaagattacatcagaagaaaggcaagtatcgcatcatttcactgcccacaaaaatacattctttccctttccctccactattttaagacttcattgaccttttctaaggtcccaatttccatttaacctctgtgccagtcaagagagcagtcccaagtctctaactttcttcacccaatgatcactctttgaactcaccaagcatgctacaaacagaaggaagccattagcagggggagttcgaacctatatagccaaccataaattagaagcccaaggaagcattttcaaccactacgaggtcactcaggctgactgctggcagtggcttctcatcgatgacag

SEQ ID NO: 224 - DNA, 401 bp, homo sapiens
gaattctggaatcagagaggagaaaccacttacctcaggggctgaaggagcctttgggcttcctggctagagtgctgaggaacttggtgctctcaggctgctggtgggaggagagacctgggtttgaggaggtgactggccaggcttatggacggggatccacatgatgaatttctccagcccctgtgcactcaaactgcggtaaaatcagttagtgggtttgcgctttgagcagctctagtaattcatttggtgtgtctttatagtttacaaggcactcttgcttctgagtttcccttggaggctgaggccactcccagaagcccagtaaagagcagtggccaagtgcacaaactgggcctgggctcagatcccagctctgtcgctttctaactgtgtga

SEQ ID NO: 225 - DNA, 401 bp, homo sapiens
  variation 292: /replace=“t”
cattgacctcccagatctgcctgtgatttggggcttatagagatgtgttgattctgtctagtcaaagtgagagtcattgcagaacaaaaccagaatcttagtggtgactgtgcaaagccatttggtctaatgattttaccaagagcatctcagaacactagtattggtagtatgtagccttgttaataaatatatacaagtacatgttgaaatgacttgttattatatggctagagcaaacccacgtcatcctcatgatgtcttgctgtcacaatctcattctctcttggggtctgacctgactcctcaagcaagagaacaagggttggcagagctctccaggaatgcacagccccaggtgcccagcagagggcatcaagtatgacagatattggaatgac

SEQ ID NO: 226 - DNA, 401 bp, homo sapiens
  variation 61: /replace=“t”
aagagcatctcagaacactagtattggtagtatgtagccttgttaataaatatatacaagcacatgttgaaatgacttgttattatatggctagagcaaacccacgtcatcctcatgatgtcttgctgtcacaatctcattctctcttggggtctgacctgactcctcaagcaagagaacaagggttggcagagctctccaggaatgcacagccccaggtgcccagcagagggcatcaagtatgacagatattggaatgacatctgttgtgtgtgcagagcagactgactgagtggaaccatgaccaaagagaactcaggtagactcaagtgcttctgggaaatttgagaaacttccctgacagagaatttacttcaagtcagatacctccccaataattg

SEQ ID NO: 227 - DNA, 401 bp, homo sapiens
  variation 268: /replace=“c”
aaatttagtaaagcaatattttattgttctagcaacaaaaaaattaagttattacattaattagaaagtgatctgagaaccttattttgatataggaggggagcagggaagtgctgggaagggaagggcatggtccctggctaggaatccatccctgggtttgtgcccatggacctaggtgaggacagggatttctgtttttgtgtccaaatgttgcatttcccaagaccgctctggccaaccatgcccccatcctgtgcctgtaaaaaccctgagacattagcaggcagagacacaagtagctggacgtcgagaggaatgtaccggcagaggaggacatcaacagacgcccgcagacaccaggccagtaggccgtcgacaaatggaactatgtggtgatg

SEQ ID NO: 228 - DNA, 401 bp, homo sapiens
  variation 134: /replace=“t”
gtgatctgagaaccttattttgatataggaggggagcagggaagtgctgggaagggaagggcatggtccctggctaggaatccatccctgggtttgtgcccatggacctaggtgaggacagggatttctgtttctgtgtccaaatgttgcatttcccaagaccgctctggccaaccatgcccccatcctgtgcctgtaaaaaccctgagacattagcaggcagagacacaagtagctggacgtcgagaggaatgtaccggcagaggaggacatcaacagacgcccgcagacaccaggccagtaggccgtcgacaaatggaactatgtggtgatggattggcaagaaccttgaatcctggacatgaccattttacaatgttattgttaaatttttttcttga

SEQ ID NO: 229 - DNA, 401 bp, homo sapiens
  variation 74: /replace=“t”
gcatggtccctggctaggaatccatccctgggtttgtgcccatggacctaggtgaggacagggatttctgtttctgtgtccaaatgttgcatttcccaagaccgctctggccaaccatgcccccatcctgtgcctgtaaaaaccctgagacattagcaggcagagacacaagtagctggacgtcgagaggaatgtaccggtagaggaggacatcaacagacgcccgcagacaccaggccagtaggccgtcgacaaatggaactatgtggtgatggattggcaagaaccttgaatcctggacatgaccattttacaatgttattgttaaatttttttcttgagcaaggtatgtataagtaagtaaataagggcataatgtcaggtaaactgaatggctggaa

SEQ ID NO: 230 - DNA, 401 bp, homo sapiens
  variation 175: /replace=“t”
ttagcagtgcctcccaccacagcagacagaaggtacatctcactctaccctctgacatcagctccctcatggaacttcctttggccaatgagccgagagcagaggcgtgagttgtgtctagccagagggttcaaatgtgcttgcgtgatttggcttggctttttgagcttcagcattctccatgggaaaatcatgccctgagtaggcattgctccttcaacctgtgtcctggaatgaagacacatggaacagtctggaacctaaaacaaagcctggagttcagctgagatcagctaaggtgtggctgggacctgtgcatatggaataaatgtttgctcttataggtaaatgcgatgtggaagttgttcattactcaacattattgcagcagaagcctggtg

SEQ ID NO: 231 - DNA, 401 bp, homo sapiens
  variation 127: /replace=“g”
tacatgtacgtgtgcttgtatgcatgggtgtgcattatgtgcgtgtttgcctgtgtgtgcatgcatgcgtgtgtgtgtgtgtgtgttctctgtggagggaggagactggagattctcttccccaacattcgaggccagtttgccaaaaatcttggtgctaactgtatgtccaggttacagatccacacgcaaatgcagactcaacaacactgaggctcatcttttgggtttctccccatcttcttcaccatgactcagttctccaagaaaaatgttgttgctactttcaggaacagtccaggatgagagcttaaggtagagtccggacatgtgcatggaagcacagccatgattctatctaattggctcaaacaaaagtcttggacagtattttggctttt

SEQ ID NO: 232 - DNA, 401 bp, homo sapiens
ggaatctgctttactcaaagcccatcgagtccaatgttaatctcatccaaacacaccctcacagcaacatctagactggtgtttgaccaaacagctgttgggtaccatagcctagccaagttgacacataaacttaatcatcatgggatcagtcccaccatgccccatgcccatctttggaccagtgttctagcctcctgactgttcaggtgagttatgggagaaaacttaggaccccatgcaaatgcagactttcttttacattgtaaataccactgacattcttgctcatccaactgggacatttttgccattcagtttgtcaagcaaatataacgatgcatattttcagcacacacttccactgtttagcagatggcacatctgaaacatggtacatg

SEQ ID NO: 233 - DNA, 401 bp, homo sapiens
gggggcgcgagagagcagcaagccgtaacagctcgcgtaaaaggcgggagctggggcttcctgggggttccctagaagcggacctcgagacaaggtcctgggtgcaagcggtttatttgggggatggctgcaggaggcacagggtaggagcagggctggcagggaagggagagaagggaggaagggcagtgaagagcacaaggatggaggggctactcccgggcctgtgtgcagagcctcagccctggctctgtcaggcagaaggagctcccgacacctctgtcatctgctggctgcggagaacctggggctgacgccgtcctccgggacctccatccagaaaagggccacagaggctgagggcgtcggggcgggcactccagggtcttattctgtttggg

SEQ ID NO: 234 - DNA, 401 bp, homo sapiens
  variation 80: /replace=“g”
ggagagcagagtgacggcctagaaaggaggttcccgagggctctggggaggcggaagaggggacgcggtgtgttgagatacggggagaagttcagagcagcatgggcatgcaggtgcctgcagaggggcccatctgggctgcaccccagcaggacagggccatggcttggtgtgggttcggctggctgcacagcttcttgtaaagtttgtccccagggtccttgggagtagaggtctttcggaaggcaccatctccagagcgtgtttctgagtaactctgaggatgggttccgggttatcactgtgtgttggaaacagcagcgcccaggctgccggaggaggctggtctccctggcaggtcctggctgtgtctagagttttgtgggtcctggagataaggc

SEQ ID NO: 235 - DNA, 401 bp, homo sapiens
  variation 372: /replace=“g”
agaggcagaggattgtagttgtgaaaagtttgtacattggagtctggctttgagtcccagttttcccgtggacaggtcatgtgaccttggcaaaataaccacctcccatgcctcaggtgcctcatctgtgaaatggggataataataacacctacttcaaataaaacaatgcaaggaatgccctagaacaaacgcctggcacatagtaagttctacccaagtatttattattataataattgtataaacatttaacgacttccttaaattgtttcctattacttgctattttgaatagtgtttcaattaatatccttgtgatatggttttgctgtgtccccaaccaaatcttatcttgagctgtaattcccacagttcccatgtatcgtgggagaaaccta

SEQ ID NO: 236 - DNA, 401 bp, homo sapiens
  variation 168: /replace=“t”
gatgtgtgtgtgcatatatgtgtgtgtgtatatatatatacacatacatatacagaaagagagagagagagtctcactctgtcttccaggctgaagtgcagtggcatgatcttggctcactgcaacttctgcctcctgggttcaagcgacttttgtgcctcagcctcctgagtagctgggactataggtgcaagttaccaggccctgctaatatttttgtatttttaatagagacagggtttcaccatgttggccaggctggtctcgaattcctgaactcaagtaatccacccacctcagcctccaaaagtcctgggattacaggcgtgacccactgtgcccagctcccaaaatgtataactaagtgagaagggaaggatgtagaatagggtgggtgtttt

SEQ ID NO: 237 - DNA, 401 bp, homo sapiens
  variation 215: /replace=“t”
ctcaggtgatcctcccacctcagcctcctggctagctgggactatagacatgtgccactatgcttagctaattttttgtatttttttgttgagacaaggttttaccatgttgcccaggctgttcttgagctgggctcaagtcatccacctggcttggcctcccaaagtgtggggattacagcatgagtttctgggcctggtctctttgctacaccttgtcagggtcttttgcaggaattccttgtggtcgttggcaggtgtcatgccttatcactggtaggcaagccttggaaagctcctgtgaagattgtcctattagtatcagtttacttttttgaaaaaatatttttattgattttagagacagaatcttgctttgtcacccaaattggagtgcagtg

SEQ ID NO: 238 - DNA, 401 bp, homo sapiens
  variation 296: /replace=“c”
aattctacttataatgttcttggcctggaagtcaaggcctcacatatcttacttcaatccactatttctggcaggacacctcaccacacttcccttgctcctgtccacatgagctcttaggccaggggcatgcttctctgtttgaccacatgcacctcctctcctgttatttctgccatttgagatgctccccttcctactgctgcattaactgaaagccacacttcatgatccagaagagatgccacctcctctgccaagctggccctgactttccaggcagaaatgatgtattaccatatggcattcattgctttacagtgaattaaagctattatttttagttgtaaaaatacattttttaaaactacaagtgtaaaatacatttgtgttgcaaaagt

SEQ ID NO: 239 - DNA, 401 bp, homo sapiens
  variation 106: /replace=“t”
tgctcctgtccacatgagctcttaggccaggggcatgcttctctgtttgaccacatgcacctcctctcctgttatttctgccatttgagatgctccccttcctaccgctgcattaactgaaagccacacttcatgatccagaagagatgccacctcctctgccaagctggccctgactttccaggcagaaatgatgtattaccatatggcattcattgctttacagtgaattaaagctattatttttagttgtaaaaatacattttttaaaactacaagtgtaaaatacatttgtgttgcaaaagtccagataatacaggaaaataaaagcttcccgttccctcagctctcttctaattttaattcttggagctattactaagagtgggggccgggcgtgg

SEQ ID NO: 240 - DNA, 401 bp, homo sapiens
  variation 137: /replace=“g”
taagcttcagaagaatcttagtagaaatgcattgtttggaatgcatatgctttggtctcaataaaaatatccggttgcagagatatttcatgtggtttgaactaacaggcaaaaaattaaaaattcattggtatacactaattcatgcaattgtatattttggaggtaagtgtaatggcataggtttgctgtaatggaactgattggaaaatgatctcttggagaaaatttattgctggtcataagatttctttggtttagaaaaatgctgggtttagtaaaatctgaggcactaaatgaaagtctgctttaataaaaatgtggatctgagaactgtggtttttagtaagtgggcatttgatccattaaaattgtattggttttattctttacgttggctt

SEQ ID NO: 241 - DNA, 401 bp, homo sapiens
  variation 262: /replace=“g”
agggcaggcaagatggatgaggcaaataactcaatgagcaagtgacggcaggcccttcaggccgccttctcatgcaattgatagttgctccttcctcacatttggtttggttccctaaatgttaaatcatctctaggataagcaggtgatagcccatgaatggagccagatgctgagatgttgttcagcttgagtggcctgcagttcattcctgaccaagctgtggtttgttcctgctggctgttggatcacctccttcaaagattcatcactggataagccactgttggccattggagctattccaccaagactgatctgaactctccctatgagacttttctttttccagtaagctccatatctgattctatgaaaagttcagcccaaatgagaggacc

SEQ ID NO: 242 - DNA, 401 bp, homo sapiens
  variation 198: /replace=“c”
agacatgcaaaatataaacacacatttcattgttattagattccacaagcataaaattattctgtcagagtgactatttggttctttctcatctctatagttatctcttcgcagacaggtaacagcctgtggaggaccacactgggtatcatttgtccggcaggtgctgcgatcctaagagggtaacattctccccagccactggaagtgggtggaatggagccactatagtaaatgttgttggatgcctacttagcagcctgcctcctgccccctgcctccttcctgagagactccaatttactcagctgtgcactggacacaccaccctgattgtggctggcctcacctgtaggttgtggcaaacttgactggcctaagggtaatcccatatgccttac

SEQ ID NO: 243 - DNA, 401 bp, homo sapiens
  variation 13: /replace=“c”
atgggaaaaaagagggtcttgataacattctaattagccccaaaccctctattcccatgagccagtctcttctctttaagttgatgctagctgccgttttgtgttatctgttacagactaatacaatttgcaataattgaagatgcaatatttattgatatattattatcaattgaagtccatagcttacattaggattcactcttagttttgtatgttctgtggattttgacatatgtaaaatgacttgtgcccaccattacagtatcacatagaatggtttcactgccccaaaataccctctgttccatctatttatccctcttcctttccatggacccttgacaaccactgatctttttattgtttccatagttttgccttttccagaatgtcatgta

SEQ ID NO: 244 - DNA, 401 bp, homo sapiens
agtttcctatgaaggcagtcgctaaagaacattccactttgcataaaagacttcatttcaaggctttcagtcaaatgcatggctagctgatggatcccactgggcttaacaagcacaattaaggtcttcatcaaaccttaatagattggtgtcattgagcactgaccagtgcttggaatgtttttcttctctgcctatcatccatgtctgaaattttcctcaaatattggcaaagagaacatataagggtggattagtagttctcctttattcatatattgtgatattttaaaggcatctttcagtaaatgcgttatcatattagcatctaactgactgatccaatgggttatatcaaaatgcaaggtgcgtacacatgggactcatatctcaaattcatt

SEQ ID NO: 245 - DNA, 401 bp, homo sapiens
  variation 313: /replace=“c”
gcctgtttctaggtccagaatggtattgcctaggttgccttctaggagttttatagatttgggttttacatttaagtctgtaatccatactgagttaatttttatatatagcataaggaaagtgtcctgtttcaatcttctgcatatggctagccagttatcctagcacaatttattgaatagggaatccattcctcattgcttgttttttgtcaggtttttcaaggatcagatagttgtatgtgtgcagccttatttctgggctctctattctgttccatcagtctatctgtctgtttctgtatcaataccatgctgttttggttactatagccctatagcatacttcaacgttgagtagtatgatgcctccagctttgttcttttgcttaggattgtct

SEQ ID NO: 246 - DNA, 401 bp, homo sapiens
  variation 310: /replace=“g”
tttaaaatagttttttctagttttgtggagaaatatcaataatggtagtttgataggaatagcattgaatctataaattgctttgggcagtatggccattataaagatattgcttgtcattatccatgagcatggaatgtttttccatttgtttgtgtcatctttgttttgtagttctccttgtagaggtctttaactttttcggttagctgtattcctaagtatttttttttcttgtggcaattgtgaatgggattgtgttcctggcttggctcttggcttgactgttgttggtatatagggatgttaatgatttttgtacattgattttgtatcctaagacttttctgaagttttgctgatgttgtttatcagcttaaggaaattttgggcttagacta

SEQ ID NO: 247 - DNA, 401 bp, homo sapiens
  variation 150: /replace=“t”
atctgctgtcctctgggcctagtttcactcccatggcagtgttagcatgagggctggatgctggtaggggcagggatggctagttctgtgtctgtccaggctccatctgtaatggtgataagaagggagagggtgggtggactgcactccttgggcaatggtgggaccaggaaagcaaaactcacctgtgcagacacatgtcagcaaagcaatgtggggagttgttgtgggcctgggggaagcagtgctttggggagggagtgggagggctggtgcatggcaactgcctcactggagttctctgctgctcaggcatggtccactgttgcagaagctatggtgcggtcccacagggcagccaagacagccctgcaagcaggcatggccaggctggggtcctg

SEQ ID NO: 248 - DNA, 401 bp, homo sapiens
  variation 31: /replace=“g”
ctggcatcctggagctcagagcagaggagaagaggtcagacagtggatctagaggggcagacaaaaatatccagaacaagtttcactctccatgggcctgaactagtggttacaattgctggaaggtagaaagtgttagggggtgttcaggtatgggatccagatgaagtgggagagaattttagtatgctttgtgaagatttcccatgatttgaaattgtgtaaggactgtcccaaattttgcaaatgatgctatatcataagataaagaagacagtcatactcatttggtagctatatttatataatgtattcatataaatgtataaatatgtatattatgtaagtggctgatgtttacatttgcttaatcatatgagaatatagcaaaattataactg

SEQ ID NO: 249 - DNA, 401 bp, homo sapiens
  variation 234: /replace=“t”
tatttcacttagcatagtgtccttcaggttcatccatgttgttgcaaatggcaggatttctttcatttgaaggctaacattccattggatgtatatgccacattttctttatccattcatctgttgataggcatttagattatttccatatcttgccttttgtgaacaatgctgcaatgaccgtggaagtactggtattcttttgagatctgatcccaatttcatttccactggatatatacccagaagtgagattcctggattgcaaggtagttctagttctataatggctacaaccaatttacattcccaccaacactgtgtggtttccttttctccacatcatcaccagaatgttttgttttattttatttttaaaataatagccaaaccatttggat

SEQ ID NO: 250 - DNA, 401 bp, homo sapiens
  variation 274: /replace=“g”
tttctgagaagtgcacgttaaacagcatagcatttcagccttgaagtgagaagtgtagactatttgtgataatgaatatacaactgaggtgttccactggagcaagtcacttagaatcacagaaagcgtattatcaggcccatgatgtaaatttaaaatgtcttattcctcattttacaaacttatgtgggcatctttccttctcacaggaagcccatttcagtgtggaaaatcagtcttgtatgaaccagatttagaacagtgccatgattcattcatattcaagagttaactgcttctacacagagttggtgtcagaaggcatgttgctgagtgccagcaactccttgggggttgagcttagatggtgtcctctgttctagcatgtgaatgctgtaaac

SEQ ID NO: 251 - DNA, 401 bp, homo sapiens
  variation 82: /replace=“t”
tgtcccactcagaggggcggttctgaggcatgctccctacagtctcccagagtggccccaggagggctaagcctcagttgcccatgatggcaatgctcattattgcaccctcgtggcttccttctctttcctgcctcatttcgtactcccaagctgtgctcccctgcatcacttcctaaactaaatcccttcatctaagaacttatcccagggtctgctttggggaagaacaaaagacaaagcacttatgatatactcaagactaacagaagcataagcttctgaggatttaaatgcaaatagaaacaatgcaatagaaatagactaaaaggcctgtgctctccttaacccacccagcctatttcagactgatgggcttggggtcagtttctgccagagtg

SEQ ID NO: 252 - DNA, 401 bp, homo sapiens
  variation 94: /replace=“g”
ttttactttttggaaagccaagcaggtcccagagtcgtggggcagtacttggcctcatctctgaggccacccattctgctgctggcagccacaatgatgtacccttggcactatgtcgctccccctacttggcatcctcagtttttctaaactcatgggtgagagacgaatgacgtcagaggcccaaacctttggatgctgatctaaaagcatcaaccaaaacccaacacccgagcttcgtaagttcccatttgaagcaggagcagtttttatagcaacagacaaaggattgctctgtccttgttggaagtgcttgaggaccaagcacagaggtgggcaaagatcgtcatcagggcttctgctacctgtcttagccccagcattcttcaaaagagatcctc

SEQ ID NO: 253 - DNA, 401 bp, homo sapiens
  variation 69: /replace=“t”
taaatttgaagtgttatggtgagatggtatcctggagacgtatgtgtgaataataaaaatgtcagcacctgctgcaaagaaccactgaatgaaaggaaaggaaaataccatacatcaaagaaagaggtttatttagattcaaagtcccctgggttaggaccacacctagtacactgtcacatccaataaatgttgagaagagactgtctagcacatgagacacagttgtggagatgtcctaggcaggctccaccacagaagcaaaacatgatccagttgcaatggaaatccactctttgctcatgtgctatttcccaagtacttgcttattaaaatataactctgctaatgaaccaaacacagggagttgctttctagagccgaaaaaaagggactgtgag

SEQ ID NO: 254 - DNA, 401 bp, homo sapiens
  variation 35: /replace=“c” /replace=“g” /replace=“t”
gttgcaatggaaatccactctttgctcatgtgctntttcccaagtacttgcttattaanatataactctgctaatgaaccaaacacagggagttgctttctagagccgaaaaaaagggactgtgagaaaaagctagggtggtctaaggagtgaattagtgacaatgtcagtggatcaactcccaacacataggttttcagacagggaattagtctaggcagaagttcatcagacccagttcttagatgtctttggtctttgtggaagatgctgttggtgccctgttcgtgtactccatgggctcttactggaagcaccatggctcattcattatcagggggctttcttgagcacactttcttgcacaaggtggggcacaggctggcatttgtaggggaggt

SEQ ID NO: 255 - DNA, 401 bp, homo sapiens
actgtcctctagggtgctaacacagtaagactggatacctccttagaccagtgcacagcatggtcaaatgcactgaatcccatccacaaagacagcctcattaactattcagaaaattgctattttgtgcttctaatctctgttaagacactgacaagaagtcattctagtcatgtgttacagacatggagatacaaagctcagattcgccttcaaggaaggacttgtggcccagatgcaggggtgtggctatcaggtgcctccagggtgtgtctcagctgcagagatcatccttcctagggtagcgtgcatctggtggctgagtgaaatggagaataaaagtcttgcagttccccctgatgcttgatgacttgcgcaatactcactctagaggtccctgt

SEQ ID NO: 256 - DNA, 401 bp, homo sapiens
  variation 35: /replace=“t”
gaggtttcatggagagtgctctcaggatccacacctgcgaaataactataataataataataataataataataataataataatagtgaaggcctggactgggtagaggaagaaactgatccaaaacaaacgtgcaatgaggcctcagccaattcctggggggcagatctggctgtgtacagcagttcctgaataatgttggttcattcgatgttctttccttataacgttgatgagaaaacaattgatttgcacctggggccaccatctgtgtatatttgcacaacctccccataactgcatgggttttctctgagtactctagtttgctctctcattccaaagtcatgcacattaggttaattggcatgtctacatggactcagtgtgagtgaatgtg

SEQ ID NO: 257 - DNA, 401 bp, homo sapiens
  variation 42: /replace=“g”
tgagaaaacaattgatttgcacctggggccaccatctgtgtatatttgcacaacctccccataactgcatgggttttctctgagtactctagtttgctctctcattccaaagtcatgcacattaggttaattggcatgtctacatggactcagtgtgagtgaatgtgagcatgggtgtgaatgtgctctgccatgagagattgtcctgttcagggctggttcccacctcactccgtgagctgctgggatgggctctggccacccatgaccatgaactggaaataagcaggtaaagaattatcttacttggttttattaatctttcttaaatgtatgtatagttcacatttatttcaatgtttaatattagaaatgttttgcctttatttagaagtgtggta

SEQ ID NO: 258 - DNA, 401 bp, homo sapiens
  variation 81: /replace=“g”
attgatttttatgtctatgcttacaccaggagcacactgttttcatttttgtagctttttagagagttttgaaatcaataagtgtctttcaattttgttcaccttttcaaaaataattttggttattcttgagtttgcacataaaatttggaatttctgaaaaaacaaaaactgagatttttatagggcttgtattgaatttttatatcagttttgggaatattgtcatcttaacaatattgagtcttccaatccatgtatatggatgtcttttcatttatttatgtctttaaaattttttttaaataatgtcctgtagttttcattaaatatcttacacttctttggtcaaatttattcttaaatatattataatttttgatattactgtaaatgatatt

SEQ ID NO: 259 - DNA, 401 bp, homo sapiens
aaattgggacaacttagtgttcgagaaatagagaaatagctcaaggagctattagataccaacccataccatgttgttttctgagggtgggagaaactagaagaaagtttgacattactctcattccatgagacaacctcaagaacacaaacccaaactatccaggtaggacttttagtttagggagaccaccaaaagaaatggagcccatttcctctggtgattcaacaagattaacagcccctcctgaagtttttgtccagtggcctagaatctaaatatgtgctcatacccagttctttgtcttcaattcccgagtcttctgtgtgactcattctgaaagtccaggtgtggaagggagaggcgcatggaggaaaaagccaggctcagaatttatattt

SEQ ID NO: 260 - DNA, 401 bp, homo sapiens
  variation 41: /replace=“g”
atgtatctatggttgagaaattcccattaaatattggggcaaggttcagatgctgtagatatttgtttacattttatcaaatgctttcttacatgtgaacccctcaatctctgagaaatcacctaagggaagccatggatctgcctcctccaggtttcattattgcatggaatgtgaggggatttctgtcttaattaatgtataagccagctcttctgagtttgggtggatatccttgaggagaaaggtagctaaaattagcagcctctactctcttctgtgttccttataaaaaaaaagagaaactgtctcatctttaagaatggaccagttatgaacttccaacctttccattaatgctttgaagcaatgatggtgctggctgatgtgcttaagtaaga

SEQ ID NO: 261 - DNA, 401 bp, homo sapiens
  variation 156: /replace=“t”
gatttaacaagaactgcatgtattccctgttatgtgcatgtaggtatgagtgtatgtgtgttatgagtgtgtatgtgtctatatgtatgtgtgtgtttgtgtctgttggggagagaaatgggataaaaaggatcagaatctcacgtagacttttcctaagtcattttccccccaaagtctcatttgtccacctctatgaaacttacttggctgagaactgctgccaaatgatccattttctaatgtgttagtaatcactacatgaatcctcttgagaaagagctgggccaggagaacttaatgacttccaaggtcacacagcaaatccagggccgagcctggttccccgctcctgttccaggctcctcccatgccaccacatcatcaagcttgacatatgt

SEQ ID NO: 262 - DNA, 401 bp, homo sapiens
aaagcatcttccaacaaaaaaataattcaggaccaggtggcttcatggctgaattctactgtactttaaaagaacacctaactctaattctcctgaaactattccaaaaattgaagaggagggaattcttcctaattcattccgtgagggcagtattgccctgatattaaagccagacaatgatgcaacaaaaaagaaaatggatgaacatagatacaaaaattctcaacaaaatactagcaaactgaatccaagggcacataaaaaaatacaccacgctcaagtggaatttatcccatggacacaaagatgattaaacgtatgcaaatcaataaatgtgatacatcttatcaacataataaatacaaaaaccatgtgatcatctcaatagatgtggaaaa

SEQ ID NO: 263 - DNA, 401 bp, homo sapiens
acgtaagtgtggcagcatctaaacaaatctgtcttcatttcaaggaagagaacgctgataatgcacatggatgttgcaacaatgtcaaattgctatgaaaatttctaaatgaattttctgctcaattgcctaccttgttgcaaattaataacaggcagttaacataactggtagtgttctgcagaccacattctgagtaatactacacaatttactgcctgcatccctagaggggatgggggaagaaataggctaaatcctaaaatgccatgatgccatattcaaatagtgttttatgaagaattgttcatttggcattcagcactgggcagtgattgaacttgagggaggcaaggagttttagaacctgtaatataccaggtaagaatttataaaaagct

SEQ ID NO: 264 - DNA, 401 bp, homo sapiens
  variation 153: /replace=“t”
tcatttggcattcagcactgggcagtgattgaacttgagggaggcaaggagttttagaacctgtaatataccaggtaagaatttataaaaagctaacagaacaggttttcataaaagtgggactgagagttttagggacatgcagaaaaaatggggatattgttaattatcccttatccttatttattacacttgtcattagttattataattaattattagtgatccttaattaatcttaaatattttatgattatttaaacatgaattttatgaatatttaaataattttgtaaatatcataaaatattttatgatatttatcaattcagttatttcagataaaagcactgaattagtagattctgaacaatcagttaagactgatcaaatactaaatc

SEQ ID NO: 265 - DNA, 401 bp, homo sapiens
  variation 129: /replace=“g”
ttgggggaatcctttccatgcctttctctttaactgatctcaggcaaaatgtttgtcctggatgtacaatacttaaaagagttgatttaaaatttcctgaaaataagttcatctcagcttttgtgtgaaaaaggaagttctatgttggctggtgaccatttcaaagctaccctcagtctcacaattagtgtgcttggcaatgctattggacattggaatgcaaatgttcacatcccaccttgagcttgcaggattttgatgcatttcctttttgttggacaaaatagtgagtattccctaagattacttccagttgcaacctgaacaaagagaaatgcatctaaatatcctgctccaaagcctttgaacttcaccaatgcataactcatgggacacttatc

SEQ ID NO: 266 - DNA, 401 bp, homo sapiens
  variation 88: /replace=“t”
ggctgtttctaagactgatgtggcttaacaaggaattttcccagaccatctctactctccacaattttcaaacaggattattactttgtctttcacctcagtgcccatgtgtttgtggatccctctagatacttgctttgctatttcttacatctctaatatgaagaattaatgtcccagttataagaactttatgtcctgtacgtagatgttctctagtcacacccctgacatgtgtgaggcctgggaagaggcccacatcccatgggcacaatatttaaaagttgtaaatcaagtgggaaagttgttaaataaaacagattctattcttctacattgtcagatattccaccttcatcatgataaaatagaaaaatatgtgcaaacaggtttttgtgtga

SEQ ID NO: 267 - DNA, 401 bp, homo sapiens
  variation 13: /replace=“g”
ctatccaatctaatctaatctaatgcaatctaatctaatctaatctaatctctatctagtctatcatctatctatctaatctatcatctatctatgtatctatctaatctatcatctatatctacatatttttcaagtgtttacatgaagacagatattatgggttggaattaatgggacagctgagactatggtttgagtcaatcagaagagcttggatgctaaatcttgctaggtaagagaaaccttgagaaagaagggtaaaaggaagtggacaaagttacataatttatttaatttgtaatgaaacacagtggatagtattaattataattttacatataatttctgggataagaaacaaaagcagttttattaggtaaaatattagtctgaaacat

SEQ ID NO: 268 - DNA, 401 bp, homo sapiens
  variation 214: /replace=“t”
cacgtaagaagtgccttttgcctcctgctatgattctgaggcctccccagccctgtggaactgtaagtccagttaaagctctttttgttcccagttttggtcatgtctttatcagcagtgtgaaaatgaactaatacggatggatagaagggtggagggatagatggatagaatgggtggactcaatcttgttgcccttctttttcttcaccccgtttctctaccctctgcaggaagagcccaccttacttctcagtctaagaaagtggcaagcactgctgggctctttccacacctgtagttcctcagttttctgaaacataggttgtgtgtttctaatctggcaaagacttgaaggaaaaaaaatcctttttaaagtcattgtaaacagatattacttt

SEQ ID NO: 269 - DNA, 401 bp, homo sapiens
  variation 39: /replace=“g”
tttgtgtactttttgattggttgtctcagtaggcatcaaatcacttgttgcggcgtgttggggggagcttgttgatgcacagatagtggggttccatcgccagggtttctggttcagaaggtctggtgtggagactgagaatttgcatttctaacaagttcctaggtgaactgcagaattacattttaagaacaactgtttggagcacccttgccttaataatgctgcagagactttctgggctggtccaggtgtcttgtggctccgcttctgctgtgtcaatttttgaacagcatgtggaggaaagatgccctctccagggtgtggtgaggagtgaggacaatgtgggcatgtctgagctgtagtgggcatgggtgattcagcaaggactgaccggacaa

SEQ ID NO: 270 - DNA, 401 bp, homo sapiens
  variation 321: /replace=“g”
tgcagttcagacacagtgggtttcctgcaactcacttttgctatggcgacttttgttatgcacagtgaacaggaagtagaaatttgcagaatataagccttcttcacaggaaacgtattttggagctcaccacaccatacaggctgctgatttaatacaaccaggatatgtgcaaatctccgaggaatggcaaatgccattcaccttcacttttttcaccttccttctcctcctcctcctcaccttcatttttcaataacccaattatttgctggtgcatataaaagtaaacagaaaatgaataagctgtggactttgaaagatagtttttgagttagaaatctgaagggggagaaaaggccacagaaacagggtaaacagttaaagcaaacattttagac

SEQ ID NO: 271 - DNA, 401 bp, homo sapiens
  variation 305: /replace=“t”
ttttgtagcagaggaatcattcttgaaggtcccaagatcctttctgctagacttcctattcttatagttgtttctaaacttaattctttctggaaaacagaataactgcaagaaaaaatctacatcctgaaagggttgaacccatcttgaaaatggcaaaatctccatcctggtgatcactggcaccatcactcccagtatagccatcactctatgggatcctgggcactgcttagcactaggaaggcgaatggctttaaattatgattcctgtcctcaagagataagtcacattgttgaaaacctgacatcaacgtacaaagattcataatcataaaaggtaactcttggtaagtcattataatcttattaattgcttctaatggtaagtcacatcaatg

SEQ ID NO: 272 - DNA, 401 bp, homo sapiens
  variation 38: /replace=“c”
tattatctttgatgcaatgttatcattatactttatgacttttatcatgctttctcttagttatttgaacataattataatggctattttgaagtctttttctcttaaatctgatgttttgtcattctcacagacaaatttatgttttctgctttttctggtatatggctcatatttttctgtttcattgcatgcctcattactttcttttggaagttggacaatttagatcatatgtattgcagcaactgtgggtagtagcccttccccaatgcttaacttgtattgttatttgcttctttatgttttagtgagtggctggattattttagcgatgtctgtttacctccaacagtggtaaacctctgaggtcgtttctcagggaggtgcagttttgagta

SEQ ID NO: 273 - DNA, 401 bp, homo sapiens
  variation 4: /replace=“t”
gaagtctgacagagagtgtgtgtggttatagtggactgtatcagttcaaagacccaaagattagctattcgtttgtgcttgggttttaaagtggagttttctgagctttctttgttcagtattcactacaattgagtagattttggggaagaaatctgtactgtatttgtgtaaaattacaaaataacactggtttatgtgtgatgttatatgttttccccaattttctttttattatcctttgtaaaattgcaaggcaatacagccttccaattacagggaccactgacaaacgtaaacagccatcaagtaccagttggcacacatctcctgtagtgcatgccattgccatcgcaccactcctcaaaaatgcacccacaaacttattcacaattacggat

SEQ ID NO: 274 - DNA, 401 bp, homo sapiens
gactgggcaatttacaaaagaaagatatttaagggacttatagttccacatggctagggaggtctcacaatcatggcagaaggcaaggaggagcaagtcatgttttccattgatggtggcagtcaaagagagagctgtgcaaggaaactcccatttttaaagccatcagatctcatgagactcgttaactatgaggggaagagtgcaggaaaaacccaaccccataattcaatcacctcccaccaggttcttcccgtcatacatgggaattgtgggagttacacttcaagatgagatttcagtggggacacagtgaaaccatatcattccaccccagacccctcccaaatctcatgtcctcacatttcaaaatcaatcatgccttaccaacagtcccccaa

SEQ ID NO: 275 - DNA, 401 bp, homo sapiens
  variation 38: /replace=“t”
tattcattaaactattccacaaataaatgtcatatgagaacttacagctttaataagaggatgaaacaactctggaaaatcagaaaagaatgaatgggaatcaactcactgatgtgcaatgatggaggatgctgagggctttaagctgcagcaacttacttaataagttcccaagcaggagagaacttggcttctacagggaattaaaaagtttgccagaacaagttggtctggagacacaggcagctcagagtcttattggtcatgctaaggagatctgagtcttagttacatgaacaatcaaaaactcctgaagagttctaattctgttgacaaattatcagatttatcttctgaaaggaacactctgacagtcttgtggaaaagaaattggaaatgct

SEQ ID NO: 276 - DNA, 401 bp, homo sapiens
  variation 45: /replace=“t”
ttggacacattgcattgcatacatttatttatcaagtcattccacaagaatgtactgattgtcttctgtgtgtcaggcattgttgcaggctctggggattcagcaatacataatttgatgatcgaatttgatgtatcttcacaacatccaagaggaactatcagataggcctttggacatatttggtctggacctcagaatgtaggtgtgaattctgagttgcaactatacatgtatgtgtcatttgtgtgtaggtgacaattgaagcttggggcaaagatgagatgctctgattcttagattctaaggtgagatcaataaataagaagaaaaaaaggagtcatctctgggactacaccttgagaatctcaagttttaatggatggcttaggaattactca

SEQ ID NO: 277 - DNA, 401 bp, homo sapiens
  variation 214: /replace=“t”
aactcaggattttgctcactattagaacttagaggaggtcccagacaccagatgctatgacagtaccacagaaaagcatcaaataaatggcttaaataaattatctgtcactcaaggaaatactcttaccatctgcagaggtaagaactcctggtctcacctctgcatgtcaaaaactccaaagagctacaagaagaaaagcttgaaactcatgttctcccaatgttcagtcttcttctcaacacaaggcatatcagattggaagctgacccctgtgtcctgacaccatggattctataaacttgctgaatgactgatggagactgatgccatcttcctaaaaaatgagccattgtggtccttagcagtgggcaataaaaaggagagctatctactgcatt

SEQ ID NO: 278 - DNA, 401 bp, homo sapiens
  variation 273: /replace=“g”
aatccattttgtctcttctgctattttggagactgctctgaaaatgtttttcatggttttgcatgactcttgggatcacgatttgcatcttaaaaaaaggagtcatggaaaatattgcaagatatgcatttggtggagagagaatgcttttcagcttgaagctatttgggggtctgatgatctgatgatctttctgttgttggagctttgagcaggcacatgctgtagagatgctcatgtgtcactggggagctggtttctgatgcgactggaacttatagatatttgataagtcatcttttccttctgatactgccgtcaattcagtgaattgaatacatacagagtctatggtaggagagcaaaagatcaaaaggcccactatgttacctccccacact

SEQ ID NO: 279 - DNA, 401 bp, homo sapiens
  variation 292: /replace=“t”
ggaaatgcccttgaagctgaggctggaaggacacggaggagctgcccagagggaacagccagtgctatttcatgatgggacagagcatggcaggtatgagcaagagaaagaggccagtgtggctggagcgtaatgcaagaaagagagtgtgacaaggtgcagctgagcaggtgggaggggtggcatcaaacggggcctaggatttacgatgtgacttgcagaacaataggaagcaccaaagattgaaagcctagtagtgacatgagtttatgatgtttaaggattcttccacctgcaattcaaggtggattggggggtcagatggggcaatagtgggtgagattttaattaggaagctccggtaacccagccaataaggatggcagcattagctatgggat

SEQ ID NO: 280 - DNA, 401 bp, homo sapiens
tggaaagtaaataggagaggagaaactcaatggcacattttcttctctttcaaacacatgttcttatttaataaattctctctcaaataaaataagcctgtagttattggtggtcagtttcatgtaataggagctactacagttttcccattaaaaaaataatacaagactgcaataaagcatatagtgagtcactttattgggtacgaagtaataaaccagtggttacaatgataacgtctatgatgtgtcttagaagaaacaaccttgattgttttgtttggtttggggtggggctttttggtttagtggcacagaaaaccaatatgggatcagaccccacccttcaaatcagtgttcttatagcaactggaaaatttcactctcagcaatgatcacaa

SEQ ID NO: 281 - DNA, 401 bp, homo sapiens
agagggttaagtttacaagcatcctatcttcatagcactcaataatagatatggctttattgtgtggtcacatccattcatctaggctggtgatataaattagtggtcaaagcattcgctttggaatcatacaagccttaatttaaattatagccccaccacttaagagataggagaccttgggaattcatgtatgttcttcgagacttaatttccttacctgcaaaattaggataataatattaatagttacattataaaattgtgaagattaactgatatatataataagcttagcacaatgtctgacacagagcataagctcaagaaatgatagctgatgttactacttttagaggtccatgtccataaaaccctttatagagaaattggaagtagga

SEQ ID NO: 282 - DNA, 401 bp, homo sapiens
  variation 41: /replace=“t”
ttaactgtgccagagaaggaaatgtacctgcaattgctcactggggtaatatttaagtcaaggctggatttcaagctcaatttaagtgctacattttgattttttttaagaagaaagatttgcctgtaaaaatgctacttttcttatacaagatctataaacatgctttgacaacctttgaaattgattaaaagtgggcttatatttctaaatggagatgccttgagtcaagatgagtaagctaagaaaatctattcattttagatatattttaactctccttactgtgcattgaaattatcaataatttccagttattctttgtttatattttggcaccattttattcccctcccctcaaaaaaaaactccaaacactaaggtgaaatgtaagcaaatag

SEQ ID NO: 283 - DNA, 401 bp, homo sapiens
  variation 76: /replace=“g”
gtaagcaaatagtactggaaattttataaattttatacatagcaatcagaagtttataaagtagatcattctgaaagcagattatgttaatcagtcaaactatttaaaagcaggagtgccaatgtgggggtaaactcaactctttcgaagagctcatttgttaacacatttatcgatatccccagttgccatctccttaatcttttgctcactcggaggaaaattcctaatagcaagatacacatgaagacagcagaacatttacaagaaagtcagccagacatgtggcagccaaagtctgtcaaagtgtagagaattttttgtatgtggcaaaatacacataacataaaattgaccattttaaccattcttaaatgtacagttaagtggcattaagcaca

SEQ ID NO: 284 - DNA, 401 bp, homo sapiens
  variation 357: /replace=“c”
ttggtgagttttcccatttctcctcttccaacaaccttattcagaagaagctgtagctacctgtttttggcagaggccagagagtttgagcaccttttcaccagctcaccaactcaatggtggactttagcaaagcattatctaattttagtacccacctgcttacccactatactataaactacaaaaagcagaacagcgtagcatttttgcataaatggtggatcaacagaccagaactgggagccctggaatgaatgtgtgttagaggaaccattgcctgtcaaggaatatgatattattcaaagatgacttacctatttggaaaaataaaacaaagcattctcatattatatgacatattaaaatacattccacctggattaaggagttatcgaaaa

SEQ ID NO: 285 - DNA, 401 bp, homo sapiens
  variation 36: /replace=“g”
aacgggatggtattctcaaacttttctgtgcataaaggttatggagagcttttaaatatccccatggcctggacacatccaagaccaataaatcgcaatctcagttattcaaatttgcaggtaatttcaatgggtggtagccaagtttgagattaaagtaaagaagcaaggttctgtaaacttttcaagtgatcccatcttgcaaacgaggaaaactgcaatctgctttttcacagccatcgtgtgtttcagcaattgcaaaatattctcagactatctctaagagtccctaagactctggtcacatcacctcctgcagcaagttgacggaaggccactatacttggtgacttttctctcatgcattcactttcacctcctagagctttcttttggaccca

SEQ ID NO: 286 - DNA, 401 bp, homo sapiens
  variation 199: /replace=“g”
ggtagcatgggttatctatgttattattatacctcttcaagcctgtcttaaaaccgccccatccaggaagttccaggttaggaaagcgtcttctttttaaactaagcctttgggcttaagatattgtcgagatctgcgaactgtgagtaatggctcattcttctccatgtgtatcttgattttttgacacaggcttcaaatcctacctaaactgcagaccaatctctgaaagcaactttgttcaattgcagcctgactaggtaaggctttgagaatttgtttctgttttaaatcctacaggataaaagaggtggagggtggtctataataggatgaagtggtatataataagtatttaacttaaaagaaaatctaccatttaaaactgtaactagattatt

SEQ ID NO: 287 - DNA, 401 bp, homo sapiens
  variation 248: /replace=“g”
tattctttttccctttgttatgtttcttccatttccaagtggatgaagctctaactcaacctttcatttgtttcaagagggaggtatcagaaccacaacaaatggaatgaaacctaccactaccacctgcctggcacaaagaaataatttacaattcacggggaatttcctgtgtttcattttgtctgaagaactgtaactggacttgaatgaactaagtgtctaattgttgcattatttttcttgtagagattttagtgttttattactttctccagtttacttcaatagtgaattaggttccctctaccatctgaaatgttacagttcgataaaataggtaaattaataaagtctaaagtgcttccaagctgagcagagacctttctgtgcgctgtgct

SEQ ID NO: 288 - DNA, 401 bp, homo sapiens
  variation 9: /replace=“g”
tttttaccataactatttttattaggttcctaagtgataagatcaggacaaagacaggttcattatgctagttagacattttcctaaagtatgataaaataaatgaaaaggaaaacaatacctgtttatctgagatagaagaagtgtttacaagttgttatgctgcattctagttgtgctaggggttacagggatctatatgtgtgtttatgctcacgggatgtacaccaaaaagaagtcaattttactgtatgttaattacaaaaagaaagctattaaatcttcctcaaatcctagaaaaatataagtacacattatttcccacacaaactcttgtgaagggttaacaaaactcatctggctttatttcatgcaagacttagaaaggctaatagtacgga

SEQ ID NO: 289 - DNA, 401 bp, homo sapiens
  variation 185: /replace=“t”
ttttattaggttcctaagtgataagatcaggacaaagacaggttcattatgctagttagacattttcctaaagtatgataaaataaatgaaaaggaaaacaatacctgtttatctgagatagaagaagtgtttacaagttgttatgctgcattctagttgtgctaggggttacagggatctatacgtgtgtttatgctcatgggatgtacaccaaaaagaagtcaattttactgtatgttaattacaaaaagaaagctattaaatcttcctcaaatcctagaaaaatataagtacacattatttcccacacaaactcttgtgaagggttaacaaaactcatctggctttatttcatgcaagacttagaaaggctaatagtacggaactgcagatcaaacca

SEQ ID NO: 290 - DNA, 401 bp, homo sapiens
  variation 281: /replace=“g”
cctccctcaaaatacttgcatctggaatttggctccttttctccctgcttaaatggtatagtgcaggtgataactgcgtagataaacctttatagttagaaagagtctaaatgttggtgtctgtaagcacaataagtaaatgagtttggtttttccttccaaaataactgaacgttcactttaaatataatgttagccagttttgaatccagaatatttacatcttgcagactgtgtcttgcagcctctgaatcaactgactgggagcatccgtctctgcatgcagggctgctgaaaggtgggagcagagataaaagacaggagatgggaagaaacaaagggagccaagagatttcatagaatgctgcaagagacattcttttgatctatttggactcctc

SEQ ID NO: 291 - DNA, 401 bp, homo sapiens
  variation 291: /replace=“t”
ttgaaaagatagtgtcaaaattgtaccacaatctggcagctcccaaattattgttgataagccaatttcagacttgtctacatatcaaaagttatttttttctcagggaaaagaagagcgtgcatgtgcaaacgcctgccactcaacaaccgtaagactatgagagggcgggactcactggaaggcagagagcctttttatttctctttctctgacctatgatgcttagccatgtgcttgttattagtggacaatcaacaaatattttctgaattaaaacccagatttgaatttttgaaacataagatttattttattaaaataagaaaatatacctagccatatacctccccaaattgaaaaaaggtattcagacaaagacttatacacaatattcatag

SEQ ID NO: 292 - DNA, 401 bp, homo sapiens
  variation 38: /replace=“t”
ccaaggtcctgaggtaacagatgtccccattaactcagccaggaggggctgaaaggcacatcagactaaagagagagagagaggtaaaaggtcctagctcctcaaactgagtgccagtggatagtgttgcacagagtgggaagagggctcatggcaggagccaaacccaagtgtcccacgggatgtgtgtcttgagacaatatggccagccccacccctcccctcatccctcagtgaaaatgacatccaagtgggacagcccagtgccatggcagcaagacagctggagctggaagcacagaggggtggagagatctctgtgggctggtggtggggccagggctggtggtggggctgctggtctatgtggtcacccccaggcacctgttgtgccatgggat

SEQ ID NO: 293 - protein, 440 aa, homo sapiens
MEQTYGEVNQLGGVFVNGRPLPNAIRLRIVELAQLGIRPCDISRQLRVSHGCVSKILARYNETGSILPGAIGGSKPRVTTPNVVKHIRDYKQGDPGIFAWEIRDRLLADGVCDKYNVPSVSSISRILRNKIGSLAQPGPYEASKQPPSQPTLPYNHIYQYPYPSPVSPTGAKMGSHPGVPGTAGHVSIPRSWPSAHSVSNILGIRTFMEQTGALAGSEGTAYSPKMEDWAGVNRTAFPATPAVNGLEKPALEADIKYTQSASTLSAVGGFLPACAYPASNQHGVYSAPGGGYLAPGPPWPPAQGPPLAPPGAGVAVHGGELAAAMTFKHPSREGSLPAPAARPRTPSVAYTDCPSRPRPPRGSSPRTRARRERQADPGAQVCAAAPAIGTGRIGGLAEEEASAGPRGARPASPQAQPCLWPDPPHFLYWSGFLGFSELGF

SEQ ID NO: 294 - DNA, 2838 bp, homo sapiens
aaattgattcccgcacgctgcagcctcccggtcagacgaatttctcccaatcggatgaagttcaccctgggcctggggtcgcgggcgtggagagtgtcctgggagggggcagcagcggcggcggcaggccctggagcgggcggcagcgcgctccgctgccgcgcacagcgcgtctccagcccgcggctgggccgccgcggctctcggctctcgggcgccctccctctatgcctctcacgcggcggcggcggcgcccaagctctcccggactgcgccgggcccagccccggccaccccggccaccccggcgccaggcagctggccggcccgctcgctatggagcagacgtatggcgaggtgaaccagctgggcggtgtgttcgtcaacggccgccccctgcccaacgccatccgcttgcgcattgtggagctggcgcagctgggcatccgaccctgtgacatcagtcggcagctccgcgtatcccacggctgcgtgagcaagatcctggcgcgctacaacgagaccggctccattctgcccggggccatcggggggagcaagccccgcgtcaccactcccaacgtggtcaagcacatccgggactacaagcaaggagaccctggcatctttgcctgggagatccgcgaccggctgctggccgacggcgtctgtgacaagtacaatgtgccttcggtgagctccatcagccgcatcctgcgcaacaagatcggcagcctggcgcagcccggaccgtacgaggcaagtaagcagccgccgtcgcagcctacgctgccctacaaccacatctaccagtacccctaccccagtcccgtgtcgcccacgggcgccaagatgggcagccaccccggggtcccgggcacggcgggccacgtcagcatcccgcgctcatggccctcggcacactcggtcagcaacatcctgggcatccggacgtttatggagcaaacaggggccctggctgggagcgaaggcaccgcttactctcccaagatggaagactgggccggcgtgaaccgcacggccttccccgccacccccgcagtgaatgggctagagaaacctgccttagaggcagacattaaatacactcagtcggcctccaccctctctgccgtgggcggctttctccccgcctgcgcctacccggcctccaaccagcacggcgtgtacagcgccccgggcggcggctacctcgccccgggcccgccgtggccgcctgcgcaaggtcctcctctggcgccccccggggccggcgtagctgtgcatggcggggaactcgcggcagcaatgaccttcaagcatcccagccgagaaggaagcctcccagctccggcagcaaggccccggacgccctcagtagcttacacggactgcccatcccggcctcgacctcctaggggcagctctccccggacccgagcccggagggaacggcaggcggacccgggcgcacaggtctgcgcggcggccccggcaatcggcacgggcaggatcggaggactcgcggaggaggaagccagtgccggcccgcggggtgcacgcccagccagcccccaggcccagccctgcctctggccggacccaccacacttcctttattggtctgggtttttaggcttctctgaacttgggttttagactgccgtaccctcctcacaatccttgctctgacgtggcctccttcgctctgccagcttcaaatttcttttttgtcactcccttgtccgtctccgtctcgcctctctccctgtttccttcccccctctttctttctcactctccctcccttccctttctctttcccttcctccctaccttccaccccggctttccccgaccttccagggctccctctgcccttccactctcttttccttgctccgacctctgctccagtcccgcttcttcccccgtctcctctttctagtcctctatatgctatcagccctttttcctggtccatccctgtcgttccttctctctctgtctctggttctactgtcaagggccctagttccctttgttttactgcttccccaaccctccctcagtccctgagagccctagagccatctcggtgacagtcccttctctggcacctgggactttgtagccttcactcctctgagcccaccagccctaatggaggggggctcaaacctaggaagtggacagaaaagccacagcctttattttgtattttttttgtaataacaaaccctaaaaaaccctccatgaactgtcctctcccgtcgatccccgcagcatattgtctggaaatctgttttctttctcagagcccagtggccgccgggcttctcttcccttctgagatgatgtcaccccaaagaagactggggtaggagtagggagcacaggggttgttggtgttttcagtgcttagaaaactgggttgtcctgcaggtgtctcctcccacccctccactcgcaacacttgctagggaatccgaccagtggtaaacacatcagcactttttggtgttcaggggtcagcacatttgttaatttaaaccacagactgcttccaggtacaaaggttcatttggacttctggtttgcaagggatgagggttaaaaaaaacaaaacaaaacaaacaacaacaacaacaaaaacaacccagaaagtatcttctagagccagccgcaagagctgtgagctgcttccaagttaggcgagctggtggtggtaccctggacataatgcaattctgtagtcagagcaatatccctggcgagagcctgacatcgcctgttaaaggtggctgcctctgaataaaggaaagcgtctgaaaggtg

SEQ ID NO: 295 - DNA, 32 bp, Artificial Sequence
  Description of Artificial Sequence Forward primer
agacatacttttctattcaccttccaatgatg

SEQ ID NO: 296 - DNA, 27 bp, Artificial Sequence
  Description of Artificial Sequence Reverse primer
ggttctgacccttattattggaactca

SEQ ID NO: 297 - DNA, 18 bp, Artificial Sequence
  Description of Artificial Sequence Probe
aagtcccaacaaactcca

SEQ ID NO: 298 - DNA, 26 bp, Artificial Sequence
  Description of Artificial Sequence Forward primer
acctgacatcaacgtacaaagattca

SEQ ID NO: 299 - DNA, 18 bp, Artificial Sequence
  Description of Artificial Sequence Reverse primer
gcggcttctcctctgcat

SEQ ID NO: 300 - DNA, 17 bp, Artificial Sequence
  Description of Artificial Sequence Probe
catggccacaagagtta

SEQ ID NO: 301 - DNA, 23 bp, Artificial Sequence
  Description of Artificial Sequence Forward primer
ggctagagaaacctgccttagag

SEQ ID NO: 302 - DNA, 15 bp, Artificial Sequence
  Description of Artificial Sequence Reverse primer
cgcccacggcagaga

SEQ ID NO: 303 - DNA, 17 bp, Artificial Sequence
  Description of Artificial Sequence Probe
acactcagtcggcctcc

(19) United States
(12) Patent Application Publication
(10) Pub. No.: US 2011/0104691 A1
(43) Pub. Date: May 5, 2011

(54) ANDROGENETIC ALOPECIA

(75) Inventors: Markus M. Nöthen, Bonn (DE); Felix F. Brockschmidt, Bonn (DE); Axel Hillmer, Singapore (SG); Roland Kruse, Dusseldorf (DE)

(73) Assignees: LIFE & BRAIN GMBH, Bonn (DE); HEINRICH-HEIN-UNIVERSITAT DUSSELDORF, Dusseldorf (DE); RHEINISCHE FRIEDRICH-WILHELMS-UNIVERSITAT, Bonn (DE)

(21) Appl. No.: 12/936,853

(22) PCT Filed: Apr. 7, 2009
(86) PCT No.: PCT/EP09/02564

§ 371 Date: Jan. 3, 2011

(30) Foreign Application Priority Data
Apr. 7, 2008 (EP) 08002933.9

(51) Int. Cl. C12Q 1/68; C07H 21/00; C12N 15/63; C12N 1/00; C12N 5/10

(52) U.S. Cl. 435/6; 536/23.1; 435/320.1; 435/243; 435/325; 435/410

(57) ABSTRACT

The present invention relates to methods for testing for a predisposition or presence of androgenetic alopecia comprising testing a sample obtained from a prospective patient or from a person suspected of carrying a predisposition for androgenetic alopecia. The invention also relates to a genetic marker for androgenetic alopecia characterised in that the genetic marker is located in a chromosomal region of human chromosome 20p11 and wherein the genetic marker is selected from the group consisting of a single nucleotide polymorphism (SNP), variable number of tandem repeat (VNTR), microsatellites or short tandem repeats (STR). Further, the invention relates to a diagnostic composition for the detection of androgenetic alopecia or a predisposition therefore as well as to a kit.

The present invention relates to methods for testing for a predisposition or presence of androgenetic alopecia comprising testing a sample obtained from a prospective patient or from a person suspected of carrying a predisposition for androgenetic alopecia. The invention also relates to a genetic marker for androgenetic alopecia characterised in that the genetic marker is located in a chromosomal region of human chromosome 20p11 and wherein the genetic marker is selected from the group consisting of a single nucleotide polymorphism (SNP), variable number of tandem repeat (VNTR), microsatellites or short tandem repeats (STR). The invention also relates to a nucleic acid molecule selected from the group consisting of (a) a nucleic acid molecule consisting of at least 17 consecutive nucleotides of any one of SEQ ID NOs:1 to 292; wherein the at least 17 consecutive nucleotides contain an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292; (b) a nucleic acid molecule the complementary strand of which hybridises under stringent conditions to the nucleic acid molecule of (a), wherein said nucleic acid molecule has at a position corresponding to position 201 of any one of SEQ ID NOs:1 to 77 an adenosine, corresponding to position 201 of any one of SEQ ID NOs:78 to 149 a guanosine, corresponding to position 201 of any one of SEQ ID NOs:150 to 219 a thymidine or corresponding to position 201 of any one of SEQ ID NOs:220 to 292 a cytidine; (c) a nucleic acid molecule identical to the nucleic acid molecule of (a) or (b) wherein each thymidine is replaced by uridine. Further, the invention also relates to a nucleic acid molecule selected from the group consisting of (a) a nucleic acid molecule consisting of at least 17 consecutive nucleotides of any one of SEQ ID NOs:1 to 292; wherein the at least 17 consecutive nucleotides contain an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; (b) a nucleic acid molecule the complementary strand of which hybridises under stringent conditions to the nucleic acid molecule of (a), wherein said nucleic acid molecule has at a position corresponding to position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264 an adenosine, corresponding to position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215 a cytidine, corresponding to position 201 of any one of SEQ ID NOs:1, 2, 4, 5, 8 to 11, 13, 14, 16, 18, 20 to 23, 26 to 28, to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 a guanosine or corresponding to position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292 a thymidine; (c) a nucleic acid molecule identical to the nucleic acid molecule of (a) or (b) wherein each thymidine is replaced by uridine. Further, the invention relates to a diagnostic composition for the detection of androgenetic alopecia or a predisposition therefore as well as to a kit.

Several documents are cited throughout the text of this specification. The disclosure content of the documents cited herein (including manufacturer's specifications, instructions, etc.), while not considered relevant for the patentability of this invention, is herewith incorporated by reference in its entirety. More specifically, all referenced documents are incorporated by reference to the same extent as if each individual document was specifically and individually indicated to be incorporated by reference.

Androgenetic alopecia (AGA, [MIM 109200]) is the most common form of hair loss in humans, occurring both in men and women. In men, this condition is commonly known as male pattern baldness (MPB), as hair is lost in a well-defined pattern, beginning above both temples. Over time, the hairline recedes and hair also thins at the crown (near the top of the head), often progressing to partial or complete baldness. In Caucasians, MBP is noticeable in about 20% of men aged 20, and increases steadily with age, so that a male in his 90s has a 90% chance of having some degree of MPB. In women, onset of androgenetic alopecia tends to occur later and in a milder form (Hanneken et al. (2003), Hautarzt 54:703-712). The pattern of hair loss in women also differs from male-pattern baldness. In women, the hair becomes thinner all over the head, and the hairline does not recede. Androgenetic alopecia in women rarely leads to total baldness.

The negative psychosocial effects associated with hair loss include decreased self-esteem, dissatisfaction with body image or appearance, self-consciousness, perception of aging, and often emotional stress. Thus, androgenetic alopecia has a considerable effect on the quality of life for many of those affected.

In addition to the psychosocial effects, recent studies indicate associations of male pattern baldness with: (1) benign prostatic hyperplasia (MIM 600082) (Hawk et al., (2000) B.I. Cancer Epidemiol Biomarkers Prey 9, 523-527); (2) coronary heart disease (Lotufo et al., (2000) J.E. Arch Intern Med 160, 165-171); (3) hyperinsulinemia; and (4) insulin-resistance-associated disorders, such as obesity (MIM 601665), hypertension (MIM 145500), and dyslipidemia (Matilainen et al, (2000) S. Lancet 356, 1165-1166). MBP is also a risk factor for clinical prostate cancer (MIM 176807) (Oh et al., (1998) Urology 51, 744-748).

The origin of androgenetic alopecia is genetic, with a high heritability of 81% (Nyholt D R, et al. (2003), J Invest Dermatol 121:1561-1564). So far, the androgen receptor gene (AR) located on the X chromosome is the only risk gene identified (Ellis, J. A., et al. (2001), J Invest Dermatol 116, 452-455; Ellis, J. A. et al. (2007), Hum Genet 121, 451-457; Hillmer et al. (2005) Am J Hum Genet 77, 140-148). Variations in the AR gene are assumed to lead to increased activity of androgen receptors in hair follicles, which can lead to a shorter cycle of hair growth and the growth of shorter and thinner strands of hair. Additionally, there is a delay in the growth of new hair to replace hair shafts that are shed.

The investigation of a large number of genetic variants covering the AR locus demonstrated that genetic variation in the AR gene is one prerequisite for the development of early-onset androgenetic alopecia, with an etiologic fraction of 0.46 (Hillmer et al. (2005) Am J Hum Genet 77, 140-148). However, the genetic variation in AR, which is located on the X chromosome, cannot explain the resemblance of fathers and sons with respect to androgenetic alopecia (Küster W and Happle R (1984), J Am Acad Dermatol 11, 921-926; Ellis, J A et al. (1998), J Invest Dermatol 110, 849-853), since sons always inherit the X chromosome from their mothers.

Thus, although androgen dependence is an important characteristic of androgenetic alopecia and genetic disposition and plays a substantial role in the development of androgenetic alopecia, androgenetic alopecia is nonetheless assumed to be polygenic, (Küster W and Happle R (1984), J Am Acad Dermatol 11, 921-926; Ellis, J A et al. (1998), J Invest Dermatol 110, 849-853; Nyholt, D R et al. (2003), J Invest Dermatol 121, 1561-1564). The psychosocial effects as well as the association of androgenetic alopecia with a variety of diseases underlines the value of understanding the molecular basis of this common form of hair loss.

The technical problem underlying the present invention was the provision of means and methods useful in the diagnosis of androgenetic alopecia or a predisposition thereto.

The solution to this technical problem is achieved by providing the embodiments characterised in the claims.

Accordingly, the present invention relates to a method for testing for a predisposition or presence of androgenetic alopecia comprising testing a sample obtained from a prospective patient or from a person suspected of carrying a predisposition for androgenetic alopecia, the method comprising determining (a) the presence and/or quantity of a nucleic acid molecule selected from the group consisting of: (i) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292; (ii) a nucleic acid molecule the complementary strand of which hybridises under stringent conditions to the nucleic acid molecule of (a)(i), wherein said nucleic acid molecule has at a position corresponding to position 201 of any one of SEQ ID NOs:1 to 77 an adenosine, corresponding to position 201 of any one of SEQ ID NOs:78 to 149 a guanosine, corresponding to position 201 of any one of SEQ ID NOs:150 to 219 a thymidine or corresponding to position 201 of any one of SEQ ID NOs:220 to 292 a cytidine; (iii) a nucleic acid molecule identical to any one of the nucleic acid molecules of (a)(i) or (a)(ii) wherein each thymidine is replaced by uridine; and/or (b) the presence and/or quantity of a nucleic acid molecule selected from the group consisting of: (i) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; (ii) a nucleic acid molecule the complementary strand of which hybridises under stringent conditions to the nucleic acid molecule of (b)(i), wherein said nucleic acid molecule has at a position corresponding to position 201 of any one of SEQ ID NOs: SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264 an adenosine, corresponding to position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215 a cytidine, corresponding to position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 a guanosine or corresponding to position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292 a thymidine; (iii) a nucleic acid molecule identical to any one of the nucleic acid molecules of (b) (i) or (b)(ii) wherein each thymidine is replaced by uridine; and/or (c) the presence and/or quantity of a nucleic acid molecule which is complementary to the nucleic acid molecule of (a) or (b); or (d) the presence of a risk allele within the nucleic acid molecule of (a) as compared to the non-risk allele, wherein said risk allele is characterised in that it is an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292; or (e) the presence of a risk allele within the nucleic acid molecule of (c) as compared to the non-risk allele, wherein said risk allele is characterised in that it is an thymidine at position 201 of any one of SEQ ID NOs:1 to 77, a cytidine at position 201 of any one of SEQ ID NOs:78 to 149, a adenosine at position 201 of any one of SEQ ID NOs:150 to 219 or a guanosine at position 201 of any one of SEQ ID NOs:220 to 292; or (f) the presence of a non-risk allele within the nucleic acid molecule of (b) as compared to the risk allele, wherein said non-risk allele is characterised in that it is an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; or (g) the presence of a non-risk allele within the nucleic acid molecule of (c) as compared to the risk allele, wherein said non-risk allele is characterised in that it is an thymidine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a guanosine at position 201 of any one of SEQ ID NOs: 3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a cytidine at position 201 of any one of SEQ ID NOs: 1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a adenosine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; or (h) the presence of a risk allele of a genetic marker of androgenetic alopecia characterised in that the genetic marker is located in a chromosomal region of human chromosome 20p11 and wherein the genetic marker is selected from the group consisting of a single nucleotide polymorphism (SNP), variable number of tandem repeat (VNTR), microsatellites or short tandem repeats (STR) and wherein the presence of said genetic marker, or said risk allele is indicative of androgenetic alopecia or a predisposition for androgenetic alopecia and wherein the presence of said non-risk allele is indicative of the absence of androgenetic alopecia or a predisposition for androgenetic alopecia.

The term “androgenetic alopecia (AGA)” as used in accordance with the present invention refers to hair loss in humans. Androgenetic alopecia ([MIM 109200]) occurs both in men and women. In men, this condition is commonly known as male pattern baldness (MPB), as hair is lost in a well-defined pattern, beginning above both temples. In women, onset of androgenetic alopecia tends to occur later and in a milder form (Hanneken et al. (2003), Hautarzt 54:703-712). The pattern of hair loss in women also differs from male-pattern baldness. In women, the hair becomes thinner all over the head, and the hairline does not recede.

Nucleic acid molecules, in accordance with the present invention, have the same bearing as understood in the art. They include DNA, such as cDNA or genomic DNA, and RNA. It is understood that the term “RNA” as used herein comprises all forms of RNA including mRNA, ncRNA (non-coding RNA), tRNA and rRNA. The term “non-coding RNA” includes siRNA (small interfering RNA), miRNA (micro RNA), rasiRNA (repeat associated RNA), snoRNA (small nucleolar RNA), and snRNA (small nuclear RNA). At the same time, other forms of RNA, including the above mentioned specific forms, are deliberately envisaged in the respective embodiments. The term nucleic acid molecule is used interchangeably herein with the terms oligonucleotide or polynucleotide.

In accordance with the present invention, the nucleic acid molecules consist most preferably of at least 17 nucleotides, but may also consist of at least 19, 25, 50, 100, 150, 200 or more nucleotides. The present invention encompasses a large number of nucleic acid molecules, which all contain as a common denominator the respective nucleotide at position 201 of any one of SEQ ID NOs:1 to 292. For example, the 17-mer of the invention may contain as a common denominator the nucleotide at position 201 of any one of SEQ ID NOs:1 to 292, wherein said nucleotide at position 201 may be located within the 17-mer at position 1, 2, 3, 4, 5, 6, 7, 8, 9, 10, 11, 12, 13, 14, 15, 16 or 17 of the 17-mer if viewed from its 5′ end.

The term “hybridises/hybridising” as used herein refers to a pairing of a nucleic acid molecule to a (partially) complementary strand of this nucleic acid molecule which thereby form a hybrid.

It is well known in the art how to perform hybridisation experiments with nucleic acid molecules, i.e. the person skilled in the art knows what hybridisation conditions s/he has to use in accordance with the present invention. Such hybridisation conditions are referred to in standard text books such as Molecular Cloning A Laboratory Manual, Cold Spring Harbor Laboratory (1989) N.Y. Preferred in accordance with the present invention are nucleic acid molecules capable of hybridising under stringent hybridisation conditions to the nucleic acid molecules of the invention that are associated with androgenetic alopecia, i.e. that do not cross hybridise to unrelated nucleic acid molecules.

Nucleic acid hybridisation will be affected by such conditions as salt concentration, temperature, or organic solvents, in addition to the base composition, length of the complementary strands and the number of nucleotide base mismatches between the hybridising nucleic acids, as will be readily appreciated by those skilled in the art. Stringent temperature conditions will generally include temperatures in excess of 30° C., typically 37° C., and preferably in excess of 45° C. Stringent salt conditions will ordinarily be less than 1000 mM, typically less than 500 mM and preferably less than 200 mM. However, the combination of parameters is much more important than the measure of any single parameter; see, e.g., Wetmur and Davidson, 1968. Probe sequences may also hybridise specifically to duplex DNA under certain conditions to form triplex or higher order DNA complexes. The preparation of such probes and suitable hybridisation conditions are well known in the art. Nucleic acid molecules capable of hybridising to the nucleic acid molecules of the invention are preferably at least 70%, more preferably at least 75%, at least 80%, at least 85%, at least 90%, at least 95% or 100% identical to the nucleic acid sequences of the invention.

Generally, stringent hybridisation conditions refer to conditions which comprise, e.g. an overnight incubation at 65° C. in 4×SSC (600 mM NaCl, 60 mM sodium citrate) followed by washing at 65° C. in 0.1×SSC for one hour. Alternatively, hybridization conditions can comprise e.g. an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulphate, and 20 μg/ml denatured, sheared salmon sperm DNA, followed by washing the filters in 0.1×SSC at about 65° C. Said conditions for hybridization are also known by a person skilled in the art as “highly stringent conditions for hybridization”. Also contemplated are nucleic acid molecules that hybridize to the oligo- or polynucleotides of the invention at lower stringency hybridization conditions (“low stringency conditions for hybridization”). Changes in the stringency of hybridization and signal detection are primarily accomplished through the manipulation of formamide concentration (lower percentages of formamide result in lowered stringency), salt conditions, or temperature. For example, lower stringency conditions include an overnight incubation at 50° C. in 4×SSC or an overnight incubation at 37° C. in a solution comprising 6×SSPE (20×SSPE=3M NaCl; 0.2M NaH₂PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve an even lower stringency, washes performed following stringent hybridization can be done at higher salt concentrations (e.g. 5×SSC). It is of note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Such modifications can generally be effected by the skilled person without further ado. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The embodiment recited herein above preferably refers to highly stringent conditions and alternatively to conditions of lower stringency.

In accordance with the present invention, the term “a nucleic acid molecule the complementary strand of which hybridises under stringent conditions to the nucleic acid molecule of . . . ” has the same bearing as understood in the art. It refers to a nucleic acid molecule being of the same strand as the nucleic acid molecule of the invention and having the above defined sequence identity with the nucleic acid molecule of the invention, thus enabling for its complementary strand to hybridise to the nucleic acid molecule of the invention. The nucleic acid molecule of this embodiment has the prescribed nucleotide in the position 201 of the corresponding nucleic acid molecule of the invention. Thus, whereas no 100% sequence identity over the entire length of said nucleic acid molecule to the nucleic acid molecule of the invention is required for its complementary strand to hybridise to the nucleic acid molecule of the invention, a 100% sequence identity is required in at least the nucleotide corresponding to the nucleotide in position 201 of the respective nucleic acid molecule of the invention.

The term “complementary nucleic acid molecule”, as used herein, refers to a nucleic acid molecule the sequence of which is uniquely fitting to (e.g. hybridising to/complementary to preferably 100%) the sequence of the nucleic acid molecule of the invention. That is, the “complementary nucleic acid molecule” will be uniquely fitting to the nucleic acid molecule of the invention having the risk-allele, where said nucleic acid molecule of the invention has an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292, but will not be fitting to the sequences containing the non-risk allele as defined in SEQ ID NOs:1 to 292 at position 201. The risk and non-risk alleles at position 201 of the respective SEQ ID numbers are shown in table 1 below. Alternatively, the “complementary nucleic acid molecule” will be uniquely fitting to the nucleic acid molecule of the invention having the non-risk allele, where said nucleic acid molecule of the invention has an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; but will not be fitting to the sequences containing the risk allele as defined in SEQ ID NOs:1 to 292 at position 201. The risk and non-risk alleles at position 201 of the respective SEQ ID numbers are shown in table 1 below. The above definition also applies to the term “complementary strand”.

As complementary nucleic acid molecules are also associated with androgenetic alopecia, they may be directly analysed for the presence of the respective complementary nucleotide at position 201 of the nucleic acid molecule of the invention, which is indicative of the presence or predisposition for androgenetic alopecia. In other words, a thymidine at position 201 of a complementary nucleic acid molecule to any one of SEQ ID NOs:1 to 77, a cytidine at position 201 of a complementary nucleic acid molecule to any one of SEQ ID NOs:78 to 149, an adenosine at position 201 of a complementary nucleic acid molecule to any one of SEQ ID NOs:150 to 219 or a guanosine at position 201 of a complementary nucleic acid molecule to any one of SEQ ID NOs:220 to 292 would be indicative of the presence or predisposition for androgenetic alopecia.

The same applies to complementary nucleic acid molecules of the nucleic acid molecule of the invention being indicative of a reduced risk for androgenetic alopecia or the absence of either androgenetic alopecia or a predisposition for this condition. Thus, a thymidine in a complementary nucleic acid molecule at a position corresponding to position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a guanosine in a complementary nucleic acid molecule at a position corresponding to position 201 of any one of SEQ ID NOs: 3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, an cytidine in a complementary nucleic acid molecule at a position corresponding to position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or an adenosine in a complementary nucleic acid molecule at a position corresponding to position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292 would be indicative for the absence of androgenetic alopecia or a predisposition therefore.

The nucleic acid molecules of the present invention are characterized as being associated with androgenetic alopecia. The sequences of any one of SEQ ID NOs:1 to 292 were uniformly chosen to contain the 200 bp upstream and the 200 bp downstream of the position of the respective risk allele or non-risk allele at the genetic locus, hence said alleles are uniformly located at position 201 of any of SEQ ID NOs:1 to 292. The nucleic acid molecules of (a) contain the respective risk alleles associated with the presence or predisposition for androgenetic alopecia, whereas the nucleic acid molecules of (b) contain the respective non-risk alleles associated with the presence or predisposition for androgenetic alopecia.

The term “risk allele” in accordance with the present invention refers to the alleles with a higher frequency in the affected group than in the control group, whereas the term “non-risk allele” refers to the alleles with higher frequency in the non affected control groups. Both the risk alleles as well as the non-risk alleles can be unambiguously identified with information of position and the sequences contained in the SEQ ID NOs:1 to 292 as provided in the present application. Furthermore, table 1 details the individual risk alleles and non-risk alleles in the corresponding SEQ ID NOs as well as their position in the chromosomal region 20p11.

TABLE 1
SNPs associated with androgenetic
alopecia on chromosome 20p11

| SEQ | | | | | Non- |
|---|---|---|---|---|---|
| ID | Name of | Position | Position of | Risk | risk |
| NO: | SNP | of SNP | sequence | allele | allele |
| 1 | rs2424407 | chr20: | chr20: 21692914- | A | G |
| | | 21693114 | 21693314 | | |
| 2 | rs6035945 | chr20: | chr20: 21702101- | A | G |
| | | 21702301 | 21702501 | | |
| 3 | rs6113363 | chr20: | chr20: 21705733- | A | C |
| | | 21705933 | 21706133 | | |
| 4 | rs2208309 | chr20: | chr20: 21719561- | A | G |
| | | 21719761 | 21719961 | | |
| 5 | rs12625990 | chr20: | chr20: 21726484- | A | G |
| | | 21726684 | 21726884 | | |
| 6 | rs6035955 | chr20: | chr20: 21728053- | A | T |
| | | 21728253 | 21728453 | | |
| 7 | rs6047633 | chr20: | chr20: 21729163- | A | C |
| | | 21729363 | 21729563 | | |
| 8 | rs6082514 | chr20: | chr20: 21729956- | A | G |
| | | 21730156 | 21730356 | | |
| 9 | rs6047636 | chr20: | chr20: 21732676- | A | G |
| | | 21732876 | 21733076 | | |
| 10 | rs6047645 | chr20: | chr20: 21748245- | A | G |
| | | 21748445 | 21748645 | | |
| 11 | rs6035959 | chr20: | chr20: 21749269- | A | G |
| | | 21749469 | 21749669 | | |
| 12 | rs6082519 | chr20: | chr20: 21749301- | A | T |
| | | 21749501 | 21749701 | | |
| 13 | rs2224367 | chr20: | chr20: 21749774- | A | G |
| | | 21749974 | 21750174 | | |
| 14 | rs4281974 | chr20: | chr20: 21751381- | A | G |
| | | 21751581 | 21751781 | | |
| 15 | rs6047649 | chr20: | chr20: 21755092- | A | T |
| | | 21755292 | 21755492 | | |
| 16 | rs1535199 | chr20: | chr20: 21766108- | A | G |
| | | 21766308 | 21766508 | | |
| 17 | rs4405823 | chr20: | chr20: 21771964- | A | T |
| | | 21772164 | 21772364 | | |
| 18 | rs12479719 | chr20: | chr20: 21798586- | A | G |
| | | 21798786 | 21798986 | | |
| 19 | rs997078 | chr20: | chr20: 21798650- | A | T |
| | | 21798850 | 21799050 | | |
| 20 | rs6047684 | chr20: | chr20: 21801564- | A | G |
| | | 21801764 | 21801964 | | |
| 21 | rs6075854 | chr20: | chr20: 21814177- | A | G |
| | | 21814377 | 21814577 | | |
| 22 | rs4815081 | chr20: | chr20: 21824820- | A | G |
| | | 21825020 | 21825220 | | |
| 23 | rs6137473 | chr20: | chr20: 21832493- | A | G |
| | | 21832693 | 21832893 | | |
| 24 | rs6113418 | chr20: | chr20: 21837118- | A | T |
| | | 21837318 | 21837518 | | |
| 25 | rs1884593 | chr20: | chr20: 21838617- | A | C |
| | | 21838817 | 21839017 | | |
| 26 | rs6082558 | chr20: | chr20: 21838963- | A | G |
| | | 21839163 | 21839363 | | |
| 27 | rs6047731 | chr20: | chr20: 21846941- | A | G |
| | | 21847141 | 21847341 | | |
| 28 | rs6082560 | chr20: | chr20: 21849900- | A | G |
| | | 21850100 | 21850300 | | |
| 29 | rs6515191 | chr20: | chr20: 21853532- | A | T |
| | | 21853732 | 21853932 | | |
| 30 | rs6113424 | chr20: | chr20: 21854580- | A | G |
| | | 21854780 | 21854980 | | |
| 31 | rs6113425 | chr20: | chr20: 21855362- | A | G |
| | | 21855562 | 21855762 | | |
| 32 | rs2328682 | chr20: | chr20: 21866088- | A | G |
| | | 21866288 | 21866488 | | |
| 33 | rs2208050 | chr20: | chr20: 21866227- | A | G |
| | | 21866427 | 21866627 | | |
| 34 | rs6047745 | chr20: | chr20: 21866854- | A | C |
| | | 21867054 | 21867254 | | |
| 35 | rs2424410 | chr20: | chr20: 21876857- | A | C |
| | | 21877057 | 21877257 | | |
| 36 | rs6035995 | chr20: | chr20: 21879483- | A | G |
| | | 21879683 | 21879883 | | |
| 37 | rs1080275 | chr20: | chr20: 21884285- | A | G |
| | | 21884485 | 21884685 | | |
| 38 | rs6047761 | chr20: | chr20: 21886082- | A | C |
| | | 21886282 | 21886482 | | |
| 39 | rs4815086 | chr20: | chr20: 21886330- | A | C |
| | | 21886530 | 21886730 | | |
| 40 | rs969921 | chr20: | chr20: 21894984- | A | T |
| | | 21895184 | 21895384 | | |
| 41 | rs2207878 | chr20: | chr20: 21899647- | A | G |
| | | 21899847 | 21900047 | | |
| 42 | rs6047768 | chr20: | chr20: 21901449- | A | G |
| | | 21901649 | 21901849 | | |
| 43 | rs1555264 | chr20: | chr20: 21909210- | A | G |
| | | 21909410 | 21909610 | | |
| 44 | rs2424427 | chr20: | chr20: 21931264- | A | G |
| | | 21931464 | 21931664 | | |
| 45 | rs1980551 | chr20: | chr20: 21933581- | A | G |
| | | 21933781 | 21933981 | | |
| 46 | rs201587 | chr20: | chr20: 21975474- | A | G |
| | | 21975674 | 21975874 | | |
| 47 | rs201593 | chr20: | chr20: 21981619- | A | G |
| | | 21981819 | 21982019 | | |
| 48 | rs913063 | chr20: | chr20: 21990218- | A | C |
| | | 21990418 | 21990618 | | |
| 49 | rs1160312 | chr20: | chr20: 21998303- | A | G |
| | | 21998503 | 21998703 | | |
| 50 | rs1887056 | chr20: | chr20: 21999611- | A | C |
| | | 21999811 | 22000011 | | |
| 51 | rs6113491 | chr20: | chr20: 22005215- | A | C |
| | | 22005415 | 22005615 | | |
| 52 | rs6113495 | chr20: | chr20: 22009287- | A | G |
| | | 22009487 | 22009687 | | |
| 53 | rs11698943 | chr20: | chr20: 22013266- | A | G |
| | | 22013466 | 22013666 | | |
| 54 | rs6036037 | chr20: | chr20: 22018325- | A | C |
| | | 22018525 | 22018725 | | |
| 55 | rs6113508 | chr20: | chr20: 22023989- | A | G |
| | | 22024189 | 22024389 | | |
| 56 | rs6036041 | chr20: | chr20: 22030248- | A | G |
| | | 22030448 | 22030648 | | |
| 57 | rs201146 | chr20: | chr20: 22036450- | A | C |
| | | 22036650 | 22036850 | | |
| 58 | rs201149 | chr20: | chr20: 22038187- | A | T |
| | | 22038387 | 22038587 | | |
| 59 | rs201150 | chr20: | chr20: 22038381- | A | T |
| | | 22038581 | 22038781 | | |
| 60 | rs6137547 | chr20: | chr20: 22057695- | A | G |
| | | 22057895 | 22058095 | | |
| 61 | rs804531 | chr20: | chr20: 22076354- | A | G |
| | | 22076554 | 22076754 | | |
| 62 | rs6113543 | chr20: | chr20: 22077123- | A | G |
| | | 220773232 | 22077523 | | |
| 63 | rs804533 | chr20: | chr20: 22077732- | A | G |
| | | 22077932 | 22078132 | | |
| 64 | rs6137561 | chr20: | chr20: 22092067- | A | G |
| | | 22092267 | 22092467 | | |
| 65 | rs721353 | chr20: | chr20: 22096201- | A | G |
| | | 22096401 | 22096601 | | |
| 66 | rs804571 | chr20: | chr20: 22099812- | A | G |
| | | 22100012 | 22100212 | | |
| 67 | rs804580 | chr20: | chr20: 22108781- | A | C |
| | | 22108981 | 22109181 | | |
| 68 | rs6137580 | chr20: | chr20: 22135493- | A | G |
| | | 22135693 | 22135893 | | |
| 69 | rs804600 | chr20: | chr20: 22137717- | A | G |
| | | 22137917 | 22138117 | | |
| 70 | rs6132492 | chr20: | chr20: 22140992- | A | G |
| | | 22141192 | 22141392 | | |
| 71 | rs6137582 | chr20: | chr20: 22145011- | A | G |
| | | 22145211 | 22145411 | | |
| 72 | rs804618 | chr20: | chr20: 22152265- | A | G |
| | | 22152465 | 22152665 | | |
| 73 | rs1090293 | chr20: | chr20: 22154956- | A | G |
| | | 22155156 | 22155356 | | |
| 74 | rs6106481 | chr20: | chr20: 22171440- | A | G |
| | | 22171640 | 22171840 | | |
| 75 | rs6113592 | chr20: | chr20: 22177305- | A | G |
| | | 22177505 | 22177705 | | |
| 76 | rs6047978 | chr20: | chr20: 22187248- | A | C |
| | | 22187448 | 22187648 | | |
| 77 | rs804688 | chr20: | chr20: 22190136- | A | G |
| | | 22190336 | 22190536 | | |
| 78 | rs6137432 | chr20: | chr20: 21701599- | G | A |
| | | 21701799 | 21701999 | | |
| 79 | rs2328672 | chr20: | chr20: 21718198- | G | A |
| | | 21718398 | 21718598 | | |
| 80 | rs1476396 | chr20: | chr20: 21718457- | G | C |
| | | 21718657 | 21718857 | | |
| 81 | rs2208310 | chr20: | chr20: 21719645- | G | T |
| | | 21719845 | 21720045 | | |
| 82 | rs2208311 | chr20: | chr20: 21721049- | G | T |
| | | 21721249 | 21721449 | | |
| 83 | rs1476398 | chr20: | chr20: 21723583- | G | A |
| | | 21723783 | 21723983 | | |
| 84 | rs2208313 | chr20: | chr20: 21724626- | G | T |
| | | 21724826 | 21725026 | | |
| 85 | rs11698973 | chr20: | chr20: 21724752- | G | T |
| | | 21724952 | 21725152 | | |
| 86 | rs2876622 | chr20: | chr20: 21725715- | G | A |
| | | 21725915 | 21726115 | | |
| 87 | rs6035954 | chr20: | chr20: 21725874- | G | A |
| | | 21726074 | 21726274 | | |
| 88 | rs6047635 | chr20: | chr20: 21729425- | G | T |
| | | 21729625 | 21729825 | | |
| 89 | rs1540930 | chr20: | chr20: 21735975- | G | C |
| | | 21736175 | 21736375 | | |
| 90 | rs6137445 | chr20: | chr20: 21737603- | G | A |
| | | 21737803 | 21738003 | | |
| 91 | rs6137446 | chr20: | chr20: 21738661- | G | A |
| | | 21738861 | 21739061 | | |
| 92 | rs6047641 | chr20: | chr20: 21739654- | G | T |
| | | 21739854 | 21740054 | | |
| 93 | rs1540932 | chr20: | chr20: 21745320- | G | A |
| | | 21745520 | 21745720 | | |
| 94 | rs2328640 | chr20: | chr20: 21751528- | G | A |
| | | 21751728 | 21751928 | | |
| 95 | rs17738464 | chr20: | chr20: 21753389- | G | C |
| | | 21753589 | 21753789 | | |
| 96 | rs2224260 | chr20: | chr20: 21756059- | G | A |
| | | 21756259 | 21756459 | | |
| 97 | rs6113394 | chr20: | chr20: 21768949- | G | T |
| | | 21769149 | 21769349 | | |
| 98 | rs6113395 | chr20: | chr20: 21769062- | G | A |
| | | 21769262 | 21769462 | | |
| 99 | rs8122229 | chr20: | chr20: 21772082- | G | A |
| | | 21772282 | 21772482 | | |
| 100 | rs2328647 | chr20: | chr20: 21778307- | G | A |
| | | 21778507 | 21778707 | | |
| 101 | rs2064773 | chr20: | chr20: 21781537- | G | A |
| | | 21781737 | 21781937 | | |
| 102 | rs1014883 | chr20: | chr20: 21792430- | G | A |
| | | 21792630 | 21792830 | | |
| 103 | rs4815080 | chr20: | chr20: 21811197- | G | A |
| | | 21811397 | 21811597 | | |
| 104 | rs6075852 | chr20: | chr20: 21813951- | G | A |
| | | 21814151 | 21814351 | | |
| 105 | rs1998076 | chr20: | chr20: 21827845- | G | A |
| | | 21828045 | 21828245 | | |
| 106 | rs6137476 | chr20: | chr20: 21845456- | G | A |
| | | 21845656 | 21845856 | | |
| 107 | rs2208054 | chr20: | chr20: 21847508- | G | A |
| | | 21847708 | 21847908 | | |
| 108 | rs2208055 | chr20: | chr20: 21847664- | G | A |
| | | 21847864 | 21848064 | | |
| 109 | rs4390829 | chr20: | chr20: 21847917- | G | A |
| | | 21848117 | 21848317 | | |
| 110 | rs2281526 | chr20: | chr20: 21856132- | G | C |
| | | 21856332 | 21856532 | | |
| 111 | rs2024885 | chr20: | chr20: 21865193- | G | A |
| | | 21865393 | 21865593 | | |
| 112 | rs2208051 | chr20: | chr20: 21866271- | G | A |
| | | 21866471 | 21866671 | | |
| 113 | rs2328684 | chr20: | chr20: 21866442- | G | T |
| | | 21866642 | 21866842 | | |
| 114 | rs6113435 | chr20: | chr20: 21868488- | G | C |
| | | 21868688 | 21868888 | | |
| 115 | rs4815087 | chr20: | chr20: 21886403- | G | C |
| | | 21886603 | 21886803 | | |
| 116 | rs201543 | chr20: | chr20: 21894728- | G | A |
| | | 21894928 | 21895128 | | |
| 117 | rs6106434 | chr20: | chr20: 21897393- | G | A |
| | | 21897593 | 21897793 | | |
| 118 | rs6047769 | chr20: | chr20: 21905849- | G | A |
| | | 21906049 | 21906249 | | |
| 119 | rs6036003 | chr20: | chr20: 21909764- | G | A |
| | | 21909964 | 21910164 | | |
| 120 | rs2424425 | chr20: | chr20: 21926909- | G | A |
| | | 21927109 | 21927309 | | |
| 121 | rs6047798 | chr20: | chr20: 21933298- | G | A |
| | | 21933498 | 21933698 | | |
| 122 | rs127747 | chr20: | chr20: 21936630- | G | A |
| | | 21936830 | 21937030 | | |
| 123 | rs201564 | chr20: | chr20: 21955402- | G | A |
| | | 21955602 | 21955802 | | |
| 124 | rs4815090 | chr20: | chr20: 21983216- | G | T |
| | | 21983416 | 21983616 | | |
| 125 | rs6113483 | chr20: | chr20: 21984324- | G | C |
| | | 21984524 | 21984724 | | |
| 126 | rs4813445 | chr20: | chr20: 21994977- | G | A |
| | | 21995177 | 21995377 | | |
| 127 | rs6113496 | chr20: | chr20: 22009432- | G | T |
| | | 22009632 | 22009832 | | |
| 128 | rs6113501 | chr20: | chr20: 22012773- | G | A |
| | | 22012973 | 22013173 | | |
| 129 | rs6113509 | chr20: | chr20: 22025152- | G | A |
| | | 22025352 | 22025552 | | |
| 130 | rs1883751 | chr20: | chr20: 22028340- | G | A |
| | | 22028540 | 22028740 | | |
| 131 | rs201145 | chr20: | chr20: 22035470- | G | A |
| | | 22035670 | 22035870 | | |
| 132 | rs6047917 | chr20: | chr20: 22052221- | G | A |
| | | 22052421 | 22052621 | | |
| 133 | rs11698068 | chr20: | chr20: 22054030- | G | A |
| | | 22054230 | 22054430 | | |
| 134 | rs804516 | chr20: | chr20: 22063529- | G | A |
| | | 22063729 | 22063929 | | |
| 135 | rs804520 | chr20: | chr20: 22066941- | G | A |
| | | 22067141 | 22067341 | | |
| 136 | rs973106 | chr20: | chr20: 22067632- | G | A |
| | | 22067832 | 22068032 | | |
| 137 | rs6113539 | chr20: | chr20: 22070929- | G | A |
| | | 22071129 | 22071329 | | |
| 138 | rs6047929 | chr20: | chr20: 22084586- | G | A |
| | | 22084786 | 22084986 | | |
| 139 | rs804573 | chr20: | chr20: 22100030- | G | A |
| | | 22100230 | 22100430 | | |
| 140 | rs6137568 | chr20: | chr20: 22110856- | G | A |
| | | 22111056 | 22111256 | | |
| 141 | rs804596 | chr20: | chr20: 22134393- | G | A |
| | | 22134593 | 22134793 | | |
| 142 | rs804598 | chr20: | chr20: 22136111- | G | A |
| | | 22136311 | 22136511 | | |
| 143 | rs12329414 | chr20: | chr20: 22139625- | G | T |
| | | 22139825 | 22140025 | | |
| 144 | rs961157 | chr20: | chr20: 22141323- | G | A |
| | | 22141523 | 22141723 | | |
| 145 | rs804610 | chr20: | chr20: 22142570- | G | A |
| | | 22142770 | 22142970 | | |
| 146 | rs804614 | chr20: | chr20: 22148214- | G | A |
| | | 22148414 | 22148614 | | |
| 147 | rs709007 | chr20: | chr20: 22151587- | G | A |
| | | 22151787 | 22151987 | | |
| 148 | rs4815098 | chr20: | chr20: 22157338- | G | A |
| | | 22157538 | 22157738 | | |
| 149 | rs1337907 | chr20: | chr20: 22162701- | G | A |
| | | 22162901 | 22163101 | | |
| 150 | rs6137433 | chr20: | chr20: 21703550- | T | C |
| | | 21703750 | 21703950 | | |
| 151 | rs6035946 | chr20: | chr20: 21706474- | T | A |
| | | 21706674 | 21706874 | | |
| 152 | rs975165 | chr20: | chr20: 21712536- | T | C |
| | | 21712736 | 21712936 | | |
| 153 | rs2208308 | chr20: | chr20: 21718708- | T | G |
| | | 21718908 | 21719108 | | |
| 154 | rs6047632 | chr20: | chr20: 21727997- | T | C |
| | | 21728197 | 21728397 | | |
| 155 | rs11906825 | chr20: | chr20: 21731905- | T | A |
| | | 21732105 | 21732305 | | |
| 156 | rs6047637 | chr20: | chr20: 21732873- | T | C |
| | | 21733073 | 21733273 | | |
| 157 | rs6137444 | chr20: | chr20: 21733439- | T | C |
| | | 21733639 | 21733839 | | |
| 158 | rs6075840 | chr20: | chr20: 21736259- | T | A |
| | | 21736459 | 21736659 | | |
| 159 | rs6137448 | chr20: | chr20: 21738808- | T | A |
| | | 21739008 | 21739208 | | |
| 160 | rs6047643 | chr20: | chr20: 21741200- | T | A |
| | | 21741400 | 21741600 | | |
| 161 | rs2180700 | chr20: | chr20: 21744655- | T | G |
| | | 21744855 | 21745055 | | |
| 162 | rs2328673 | chr20: | chr20: 21747492- | T | A |
| | | 21747692 | 21747892 | | |
| 163 | rs6047646 | chr20: | chr20: 21748310- | T | G |
| | | 21748510 | 21748710 | | |
| 164 | rs6035957 | chr20: | chr20: 21748385- | T | C |
| | | 21748585 | 21748785 | | |
| 165 | rs16983003 | chr20: | chr20: 21751114- | T | A |
| | | 21751314 | 21751514 | | |
| 166 | rs17665823 | chr20: | chr20: 21752141- | T | C |
| | | 21752341 | 21752541 | | |
| 167 | rs12624315 | chr20: | chr20: 21752928- | T | G |
| | | 21753128 | 21753328 | | |
| 168 | rs6082520 | chr20: | chr20: 21759918- | T | C |
| | | 21760118 | 21760318 | | |
| 169 | rs6113393 | chr20: | chr20: 21762992- | T | C |
| | | 21763192 | 21763392 | | |
| 170 | rs6106421 | chr20: | chr20: 21769017- | T | C |
| | | 21769217 | 21769417 | | |
| 171 | rs6047663 | chr20: | chr20: 21769710- | T | C |
| | | 21769910 | 21770110 | | |
| 172 | rs6047666 | chr20: | chr20: 21774562- | T | C |
| | | 21774762 | 21774962 | | |
| 173 | rs6047676 | chr20: | chr20: 21790689- | T | C |
| | | 21790889 | 21791089 | | |
| 174 | rs1014885 | chr20: | chr20: 21792627- | T | C |
| | | 21792827 | 21793027 | | |
| 175 | rs2180439 | chr20: | chr20: 21800900- | T | C |
| | | 21801100 | 21801300 | | |
| 176 | rs6047685 | chr20: | chr20: 21803704- | T | A |
| | | 21803904 | 21804104 | | |
| 177 | rs6035971 | chr20: | chr20: 21803948- | T | A |
| | | 21804148 | 21804348 | | |
| 178 | rs6035979 | chr20: | chr20: 21822972- | T | G |
| | | 21823172 | 21823372 | | |
| 179 | rs4544515 | chr20: | chr20: 21833111- | T | C |
| | | 21833311 | 21833511 | | |
| 180 | rs1884588 | chr20: | chr20: 21833300- | T | C |
| | | 21833500 | 21833700 | | |
| 181 | rs6113416 | chr20: | chr20: 21834162- | T | C |
| | | 21834362 | 21834562 | | |
| 182 | rs6047715 | chr20: | chr20: 21835066- | T | A |
| | | 21835266 | 21835466 | | |
| 183 | rs1884592 | chr20: | chr20: 21838490- | T | C |
| | | 21838690 | 21838890 | | |
| 184 | rs6113431 | chr20: | chr20: 21864156- | T | A |
| | | 21864356 | 21864556 | | |
| 185 | rs2328680 | chr20: | chr20: 21865906- | T | C |
| | | 21866106 | 21866306 | | |
| 186 | rs1007169 | chr20: | chr20: 21866110- | T | G |
| | | 21866310 | 21866510 | | |
| 187 | rs6047744 | chr20: | chr20: 21866698- | T | G |
| | | 21866898 | 21867098 | | |
| 188 | rs2009652 | chr20: | chr20: 21920381- | T | C |
| | | 21920581 | 21920781 | | |
| 189 | rs6036007 | chr20: | chr20: 21922390- | T | C |
| | | 21922590 | 21922790 | | |
| 190 | rs201548 | chr20: | chr20: 21922635- | T | C |
| | | 21922835 | 21923035 | | |
| 191 | rs6106438 | chr20: | chr20: 21922826- | T | C |
| | | 21923026 | 21923226 | | |
| 192 | rs6113456 | chr20: | chr20: 21924894- | T | G |
| | | 21925094 | 21925294 | | |
| 193 | rs2424426 | chr20: | chr20: 21928525- | T | C |
| | | 21928725 | 21928925 | | |
| 194 | rs201562 | chr20: | chr20: 21947974- | T | G |
| | | 21948174 | 21948374 | | |
| 195 | rs201563 | chr20: | chr20: 21948081- | T | C |
| | | 21948281 | 21948481 | | |
| 196 | rs201565 | chr20: | chr20: 21957135- | T | C |
| | | 21957335 | 21957535 | | |
| 197 | rs201569 | chr20: | chr20: 21960662- | T | C |
| | | 21960862 | 21961062 | | |
| 198 | rs201571 | chr20: | chr20: 21961314- | T | C |
| | | 21961514 | 21961714 | | |
| 199 | rs201572 | chr20: | chr20: 21962554- | T | C |
| | | 21962754 | 21962954 | | |
| 200 | rs201594 | chr20: | chr20: 21982471- | T | C |
| | | 21982671 | 21982871 | | |
| 201 | rs6047844 | chr20: | chr20: 21985375- | T | C |
| | | 21985575 | 21985775 | | |
| 202 | rs6036026 | chr20: | chr20: 21992005- | T | C |
| | | 21992205 | 21992405 | | |
| 203 | rs4813446 | chr20: | chr20: 21997559- | T | C |
| | | 21997759 | 21997959 | | |
| 204 | rs6113524 | chr20: | chr20: 22041992- | T | A |
| | | 22042192 | 22042392 | | |
| 205 | rs1303872 | chr20: | chr20: 22058834- | T | C |
| | | 22059034 | 22059234 | | |
| 206 | rs6113548 | chr20: | chr20: 22084639- | T | C |
| | | 22084839 | 22085039 | | |
| 207 | rs6113551 | chr20: | chr20: 22087734- | T | A |
| | | 22087934 | 22088134 | | |
| 208 | rs6137567 | chr20: | chr20: 22110656- | T | C |
| | | 22110856 | 22111056 | | |
| 209 | rs767457 | chr20: | chr20: 22133132- | T | C |
| | | 22133332 | 22133532 | | |
| 210 | rs804609 | chr20: | chr20: 22142275- | T | C |
| | | 22142475 | 22142675 | | |
| 211 | rs6113583 | chr20: | chr20: 22142865- | T | C |
| | | 22143065 | 22143265 | | |
| 212 | rs2876633 | chr20: | chr20: 22145486- | T | A |
| | | 22145686 | 22145886 | | |
| 213 | rs804616 | chr20: | chr20: 22149639- | T | C |
| | | 22149839 | 22150039 | | |
| 214 | rs804617 | chr20: | chr20: 22151280- | T | G |
| | | 22151480 | 22151680 | | |
| 215 | rs1090292 | chr20: | chr20: 22154769- | T | C |
| | | 22154969 | 22155169 | | |
| 216 | rs1415801 | chr20: | chr20: 22159214- | T | G |
| | | 22159414 | 22159614 | | |
| 217 | rs1337908 | chr20: | chr20: 22189266- | T | A |
| | | 22189466 | 22189666 | | |
| 218 | rs10485630 | chr20: | chr20: 22193667- | T | G |
| | | 22193867 | 22194067 | | |
| 219 | rs6047982 | chr20: | chr20: 22194250- | T | G |
| | | 22194450 | 22194650 | | |
| 220 | rs6047613 | chr20: | chr20: 21694105- | C | G |
| | | 21694305 | 21694505 | | |
| 221 | rs6082508 | chr20: | chr20: 21716842- | C | A |
| | | 21717042 | 21717242 | | |
| 222 | rs2224366 | chr20: | chr20: 21719709- | C | G |
| | | 21719909 | 21720109 | | |
| 223 | rs6047628 | chr20: | chr20: 21722138- | C | G |
| | | 21722338 | 21722538 | | |
| 224 | rs6047630 | chr20: | chr20: 21722642- | C | G |
| | | 21722842 | 21723042 | | |
| 225 | rs2208312 | chr20: | chr20: 21724535- | C | T |
| | | 21724735 | 21724935 | | |
| 226 | rs2208314 | chr20: | chr20: 21724675- | C | A |
| | | 21724875 | 21725075 | | |
| 227 | rs6082512 | chr20: | chr20: 21728705- | C | T |
| | | 21728905 | 21729105 | | |
| 228 | rs6082513 | chr20: | chr20: 21728772- | C | A |
| | | 21728972 | 21729172 | | |
| 229 | rs6113378 | chr20: | chr20: 21728832- | C | T |
| | | 21729032 | 21729232 | | |
| 230 | rs873137 | chr20: | chr20: 21731931- | C | A |
| | | 21732131 | 21732331 | | |
| 231 | rs6113381 | chr20: | chr20: 21732750- | C | T |
| | | 21732950 | 21733150 | | |
| 232 | rs6113382 | chr20: | chr20: 21735368- | C | A |
| | | 21735568 | 21735768 | | |
| 233 | rs4815066 | chr20: | chr20: 21737875- | C | A |
| | | 21738075 | 21738275 | | |
| 234 | rs16982990 | chr20: | chr20: 21738782- | C | T |
| | | 21738982 | 21739182 | | |
| 235 | rs1540931 | chr20: | chr20: 21745149- | C | A |
| | | 21745349 | 21745549 | | |
| 236 | rs2328642 | chr20: | chr20: 21753961- | C | G |
| | | 21754161 | 21754361 | | |
| 237 | rs11696327 | chr20: | chr20: 21757209- | C | T |
| | | 21757409 | 21757609 | | |
| 238 | rs2224261 | chr20: | chr20: 21761835- | C | T |
| | | 21762035 | 21762235 | | |
| 239 | rs6047657 | chr20: | chr20: 21761930- | C | A |
| | | 21762130 | 21762330 | | |
| 240 | rs1014884 | chr20: | chr20: 21792494- | C | T |
| | | 21792694 | 21792894 | | |
| 241 | rs6047677 | chr20: | chr20: 21795279- | C | G |
| | | 21795479 | 21795679 | | |
| 242 | rs987050 | chr20: | chr20: 21799849- | C | A |
| | | 21800049 | 21800249 | | |
| 243 | rs6047683 | chr20: | chr20: 21800993- | C | A |
| | | 21801193 | 21801393 | | |
| 244 | rs1012184 | chr20: | chr20: 21804714- | C | T |
| | | 21804914 | 21805114 | | |
| 245 | rs6047688 | chr20: | chr20: 21807007- | C | G |
| | | 21807207 | 21807407 | | |
| 246 | rs4815077 | chr20: | chr20: 21807443- | C | T |
| | | 21807643 | 21807843 | | |
| 247 | rs6113404 | chr20: | chr20: 21811625- | C | T |
| | | 21811825 | 21812025 | | |
| 248 | rs6075855 | chr20: | chr20: 21814347- | C | T |
| | | 21814547 | 21814747 | | |
| 249 | rs6035978 | chr20: | chr20: 21822939- | C | T |
| | | 21823139 | 21823339 | | |
| 250 | rs6047705 | chr20: | chr20: 21823455- | C | T |
| | | 21823655 | 21823855 | | |
| 251 | rs1884589 | chr20: | chr20: 21833419- | C | A |
| | | 21833619 | 21833819 | | |
| 252 | rs6047732 | chr20: | chr20: 21847048- | C | G |
| | | 21847248 | 21847448 | | |
| 253 | rs3818179 | chr20: | chr20: 21848923- | C | A |
| | | 21849123 | 21849323 | | |
| 254 | rs3818182 | chr20: | chr20: 21849198- | C | A |
| | | 21849398 | 21849598 | | |
| 255 | rs927059 | chr20: | chr20: 21861994- | C | T |
| | | 21862194 | 21862394 | | |
| 256 | rs2328681 | chr20: | chr20: 21866013- | C | T |
| | | 21866213 | 21866413 | | |
| 257 | rs2328683 | chr20: | chr20: 21866247- | C | T |
| | | 21866447 | 21866647 | | |
| 258 | rs4813442 | chr20: | chr20: 21887456- | C | T |
| | | 21887656 | 21887856 | | |
| 259 | rs970616 | chr20: | chr20: 21899854- | C | A |
| | | 21900054 | 21900254 | | |
| 260 | rs1555265 | chr20: | chr20: 21909272- | C | T |
| | | 21909472 | 21909672 | | |
| 261 | rs169311 | chr20: | chr20: 21910133- | C | A |
| | | 21910333 | 21910533 | | |
| 262 | rs6047790 | chr20: | chr20: 21926295- | C | T |
| | | 21926495 | 21926695 | | |
| 263 | rs1980552 | chr20: | chr20: 21933805- | C | T |
| | | 21934005 | 21934205 | | |
| 264 | rs6047799 | chr20: | chr20: 21934112- | C | A |
| | | 21934312 | 21934512 | | |
| 265 | rs201559 | chr20: | chr20: 21944915- | C | T |
| | | 21945115 | 21945315 | | |
| 266 | rs201561 | chr20: | chr20: 21947013- | C | G |
| | | 21947213 | 21947413 | | |
| 267 | rs201574 | chr20: | chr20: 21963364- | C | T |
| | | 21963564 | 21963764 | | |
| 268 | rs6137526 | chr20: | chr20: 21986526- | C | T |
| | | 21986726 | 21986926 | | |
| 269 | rs6132477 | chr20: | chr20: 21988222- | C | T |
| | | 21988422 | 21988622 | | |
| 270 | rs6137527 | chr20: | chr20: 21991721- | C | T |
| | | 21991921 | 21992121 | | |
| 271 | rs6036029 | chr20: | chr20: 21996977- | C | T |
| | | 21997177 | 21997377 | | |
| 272 | rs6137533 | chr20: | chr20: 22018463- | C | T |
| | | 22018663 | 22018863 | | |
| 273 | rs4815092 | chr20: | chr20: 22029133- | C | G |
| | | 22029333 | 22029533 | | |
| 274 | rs6113534 | chr20: | chr20: 22065578- | C | G |
| | | 22065778 | 22065978 | | |
| 275 | rs708996 | chr20: | chr20: 22069958- | C | G |
| | | 22070158 | 22070358 | | |
| 276 | rs804524 | chr20: | chr20: 22070610- | C | T |
| | | 22070810 | 22071010 | | |
| 277 | rs1415798 | chr20: | chr20: 22078329- | C | G |
| | | 22078529 | 22078729 | | |
| 278 | rs1415799 | chr20: | chr20: 22078994- | C | T |
| | | 22079194 | 22079394 | | |
| 279 | rs2328701 | chr20: | chr20: 22083801- | C | G |
| | | 22084001 | 22084201 | | |
| 280 | rs804553 | chr20: | chr20: 22092575- | C | T |
| | | 22092775 | 22092975 | | |
| 281 | rs6106474 | chr20: | chr20: 22136598- | C | T |
| | | 22136798 | 22136998 | | |
| 282 | rs804605 | chr20: | chr20: 22139857- | C | T |
| | | 22140057 | 22140257 | | |
| 283 | rs804606 | chr20: | chr20: 22140246- | C | T |
| | | 22140446 | 22140646 | | |
| 284 | rs6082642 | chr20: | chr20: 22144220- | C | G |
| | | 22144420 | 22144620 | | |
| 285 | rs742655 | chr20: | chr20: 22147303- | C | T |
| | | 22147503 | 22147703 | | |
| 286 | rs804615 | chr20: | chr20: 22148975- | C | T |
| | | 22149175 | 22149375 | | |
| 287 | rs709006 | chr20: | chr20: 22151540- | C | T |
| | | 22151740 | 22151940 | | |
| 288 | rs12625314 | chr20: | chr20: 22160746- | C | T |
| | | 22160946 | 22161146 | | |
| 289 | rs12625315 | chr20: | chr20: 22160762- | C | T |
| | | 22160962 | 22161162 | | |
| 290 | rs1337906 | chr20: | chr20: 22162621- | C | T |
| | | 22162821 | 22163021 | | |
| 291 | rs804684 | chr20: | chr20: 22182536- | C | T |
| | | 22182736 | 22182936 | | |
| 292 | rs6113604 | chr20: | chr20: 22200075- | C | T |
| | | 22200275 | 22200475 | | |

The term “genetic marker” in accordance with the present invention refers to a DNA sequence at a genomic locus that varies due to mutation and that is associated with a disease. A genetic marker may be a short DNA sequence, such as a sequence surrounding a single base-pair change (single nucleotide polymorphism), or a long one, like for example microsatellites.

The term single nucleotide polymorphism (SNP) in accordance with the present invention refers to a DNA sequence variation occurring when a single nucleotide—A, T, C, or G—in the genome differs between paired chromosomes in an individual or between members of a species. Almost all common SNPs have only two alleles. For a variation to be considered a SNP, it must occur in at least 1% of the population. Within a population, SNPs can be assigned a minor allele frequency, which is the lowest allele frequency at a locus that is observed in a particular population. Single nucleotide polymorphisms may fall within coding sequences of genes, non-coding regions of genes, or in the intergenic regions between genes. SNPs within a coding sequence will not necessarily change the amino acid sequence of the protein that is produced, due to degeneracy of the genetic code. A SNP in which both forms lead to the same polypeptide sequence is termed synonymous or a silent mutation. If a different polypeptide sequence is produced they are non-synonymous. SNPs that are not in protein-coding regions nonetheless can affect gene splicing, transcription factor binding, or the sequence of non-coding RNA.

The term “variable number of tandem repeats (VNTR)” refers to short nucleotide sequences generally ranging from 14 to 100 nucleotides organised into clusters of tandem repeats that are usually repeated in the range of between 4 and 40 times per occurrence. Clusters of such repeats are scattered on chromosomes and each variant constitutes an allele.

The term “microsatellites” or “short tandem repeats” (STR) relates to polymorphic loci present in nuclear and organellar DNA consisting typically of repeating units of 1 to 10 base pairs in length. The repeated sequences are directly adjacent to each other and in the same orientation. Microsatellites and STRs are characterised by a high allelic variability due to an increased rate of mutation compared to other neutral regions of DNA.

Using a genome-wide association study (GWAS) in 296 individuals with androgenetic alopecia (male pattern baldness) and 347 controls, the genetic basis of androgenetic alopecia was investigated in a genome-wide context. The chromosome 20p11 locus was found to have a strong effect on the development of early onset androgenetic alopecia. Analysis of two of the associated SNPs on chromosome 20p11 (rs2180439 and rs6113491) for interaction with the primary associated SNP at the AR locus (rs1041668) did not provide evidence for an interaction with the AR locus (see Example 5). The chromosome 20p11 locus, therefore, shows no evident connection to the androgen pathway. Thus, the present inventors identified a chromosomal region associated with androgenetic alopecia. Within this region, 52 SNPs have been shown to be associated with androgenetic alopecia (P<0.05). These SNPs are located at position 201 of any one of SEQ ID NOs: 1, 7, 23, 27, 30, 36, 39, 41, 42, 43, 51, 53, 60, 61, 62, 74, 77, 86, 92, 103, 104, 105, 106, 111, 116, 117, 118, 121, 132, 136, 141, 145, 153, 156, 157, 166, 175, 183, 198, 200, 208, 215, 255, 257, 258, 259, 270, 278, 286, 289, 290, and 292. The human genome has been analyzed in detail by the Human HapMap Project and the correlation between the alleles of two SNPs (at high correlation two SNPs are in linkage disequilibrium) in a genomic region is well known and the information is publicly available at www.hapmap.org. By combining the association data with the publicly available linkage disequilibrium data the inventors defined 240 additionally SNPs which are in linkage disequilibrium with at least one of the 52 associated SNPs. The risk alleles of the 240 SNPs are clearly defined by the risk alleles of the 52 associated SNPs and can therefore be used as a substitute of these. Any of the genetic markers within the chromosomal region can thus be used by the skilled person to determine the presence or predisposition for androgenetic alopecia.

Due to the abundance of established methods for assessing the genotypes of SNPs, it is now possible to conveniently diagnose the presence or a genetic predisposition for androgenetic alopecia in a short amount of time, at low cost, with high accuracy and without significant trouble for the person under investigation. Detection of the non-risk allele may also conveniently be used to demonstrate that a person does not have androgenetic alopecia or a predisposition therefore.

Methods for testing a sample for the presence of the nucleic acid molecule of the invention, for the presence of said genetic marker or for the presence of said risk or non-risk allele include, but are not limited to, nucleic acid amplification and oligonucleotide extension, sequencing or hybridization assays.

Examples for nucleic acid amplification assays and means to perform such include without limitation PCR, (including nested PCR, RT-PCR, PCR extension assays, Nucleic Acid Sequence Base Amplification (NASBA), single-strand confirmation polymorphism (SSCP) PCR), amplification refractory mutation systems (ARMSTM) and amplification refractory mutation system linear extension (ALEXTM) assays. Details of such methods can be found in art, see, for example, Newton et al., Nucleic Acids Res. 17 (1989) 2503-2516; Agrawal (Ed.), “Protocols for Oligonucleotides and Analogs: Synthesis and Properties (Methods in Molecular Biology, 20)”, Humana Press, 1993; Hague et al., Diagn. Mol. Pathol. 7 (1998) 248-252; Innis et al. (Ed.), “PCR Applications: Protocols for Functional Genomics”, Academic Press, 1999; Chen and Janes (Ed.), “PCR Cloning Protocols: From Molecular Cloning to Genetic”, 2nd edition, Humana Press, 2002; Pissard et al., Clin. Chem. 48 (2002) 769-772; Blondal et al., Nucleic Acids Res 31 (2003) e155; Steemers et al., Nature Meth. 3 (2006) 31-33; Kakavas et al., J. Clin. Lab. Anal. 20 (2006) 1-7.

Examples for sequencing assays comprise without limitation approaches of sequence analysis by direct sequencing, fluorescent SSCP in an automated DNA sequencer and Pyrosequencing. These procedures are common in the art, see e.g. Adams et al. (Ed.), “Automated DNA Sequencing and Analysis”, Academic Press, 1994; Alphey, “DNA Sequencing: From Experimental Methods to Bioinformatics”, Springer Verlag Publishing, 1997; Ramon et al., J. Transl. Med. 1 (2003) 9; Meng et al., J. Clin. Endocrinol. Metab. 90 (2005) 3419-3422.

Examples for hybridization assays comprise without limitation Northern and Southern blot assays, heteroduplex analysis, detection of mutations by sequence specific oligonucleotide hybridization, allele-specific oligonucleotide hybridization on DNA chips, assays based on IIlumina's® technology, assays based on the BeadArray® technology, see, for example, Barnes et al., Nucleic Acids Res. 33 (2005) 5914-5923; Fan et al., Biotechniques 39 (2005) 583-588; Shen et al., Mutat. Res.-Fund. Mol. M. 573 (2005) 70-82; Steemers and Gunderson, Pharmacogenomics, 6 (2005) 777-782.

In a preferred embodiment said determining of the presence of said genetic marker, said nucleic acid molecule or the presence of said risk or non-risk allele comprises PCR based techniques, RFLP- or AFLP based techniques, DNA sequencing-based techniques, hybridization-based techniques, single-strand conformation polymorphism analysis (SSCA), denaturating gradient gel electrophoresis (DGGE), mismatch cleavage detection, heteroduplex analysis, primer extension-based techniques, and 5′-nuclease assay-based techniques. Said techniques are well known to the person skilled in the art.

In a preferred embodiment, the method further comprises detecting an alteration in the expression of a nucleic acid molecule encoding a polypeptide having PAX1 function, wherein said nucleic acid molecule comprises (a) a nucleic acid molecule encoding a polypeptide having the amino acid sequence of SEQ ID NO:293; (b) a nucleic acid molecule having the DNA sequence of SEQ ID NO:294; (c) a nucleic acid molecule having the sequence of SEQ ID NO:294, wherein each thymidine is replaced by uridine; wherein the alteration of expression as compared to the amount of nucleic acid molecule expression in the control sample is indicative of androgenetic alopecia or a predisposition for androgenetic alopecia.

The transcriptional activator PAX1 (paired box 1) is a member of the paired box-containing class of developmental control genes and is located in the chromosomal region 20p11.2. PAX1 is involved in the generation of the vertebral column.

The term “polypeptide” as used herein has the same bearing as understood in the art. It describes molecules having linear molecular chains of amino acids and containing more than 20 amino acids. Polypeptides may further form oligomers consisting of at least two identical or different molecules. The corresponding higher order structures of such multimers are, correspondingly, termed homo- or heterodimers, homo- or heterotrimers etc. Also encompassed by the term “polypeptide” are proteins as well as fragments of proteins, wherein the term “fragment of proteins” refers to a portion of a protein comprising at least the amino acid residues necessary to maintain the biological activity of the protein.

The term “a polypeptide having PAX1 function” in accordance with the present invention refers to a polypeptide having or essentially having PAX1 function, wherein said function can be determined e.g. by its trans-activating capacity in reporter gene assays or by its activation of NKX3-2 (BAPX1), a downstream gene of PAX1.

The expression levels of a nucleic acid molecule encoding a polypeptide having PAX1 function can be measured by any method known in the art that can provide quantitative information regarding the levels to be measured. The methods preferably are highly sensitive and provide reproducible results. In particular, methods based upon the polymerase chain reaction (real-time PCR) and related amplification technologies, such as NASBA and other isothermal amplification technologies, may be used. A suitable approach may be, for example, real-time PCR employing the relative quantification approach using GAPDH or β-actin as housekeeping gene.

Amounts of purified protein in solution can be determined by physical methods, e.g. photometry. Methods of quantifying a particular protein in a mixture rely on specific binding, e.g of antibodies. Specific detection and quantitation methods exploiting the specificity of antibodies comprise immunohistochemistry (in situ) and surface plasmon resonance. Western blotting combines separation of a mixture of proteins by electrophoresis and specific detection with antibodies.

The present inventors found that PAX1 is located in proximity to the locus identified to be associated with androgenetic alopecia. Furthermore, as shown in Example 6, PAX1 was found to be expressed at considerable levels in the scalp. These expression data are compatible with a role of this gene in the development of androgenetic alopecia.

In a further preferred embodiment, the sample is blood, serum, plasma, fetal tissue, saliva, urine, mucosal tissue, mucus, vaginal tissue, fetal tissue obtained from the vagina, skin, hair, hair follicle or another human tissue.

The present invention also relates to a genetic marker for androgenetic alopecia characterised in that the genetic marker is located in a chromosomal region of human chromosome 20p11 and wherein the genetic marker is selected from the group consisting of a single nucleotide polymorphism (SNP), variable number of tandem repeat (VNTR), microsatellites or short tandem repeats (STR).

As described above, any of the genetic markers within the chromosomal region can thus be used for determining the presence of or predisposition for androgenetic alopecia. Thus, preferably the genetic marker of the invention is for use in the diagnosis of androgenetic alopecia.

In a preferred embodiment of the method of the invention or the genetic marker of the invention, the chromosomal region of human chromosome 20p11 consists of the region having the sequence of SEQ ID NO:1 at the 5′ end and the sequence of SEQ ID NO:292 at the 3′ end.

Thus, the chromosomal region is enclosed between the sequence as defined by SEQ ID NO:1 at the 5′ end and the sequence as defined by SEQ ID NO:292 at the 3′ end. In other terms, the chromosomal region spans from position chr20:21692914 to position chr20:22200475 (the genomic positions are based on NCBI build 36).

In a more preferred embodiment, the chromosomal region of human chromosome 20p11 comprises at least one of SEQ ID NOs:1 to 292.

In a further preferred embodiment, the genetic marker is a SNP selected from the group consisting of the SNPs comprised in any one of SEQ ID NOs:1 to 292.

SEQ ID NOs:1 to 292 and the respective SNPs have been summarised in table 1 above. Table 1 further details the position of the respective SNPs in the chromosomal region 20p11 and the corresponding alleles.

Further, the invention relates to a nucleic acid molecule selected from the group consisting of (a) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292; (b) a nucleic acid molecule the complementary strand of which hybridises under stringent conditions to the nucleic acid molecule of (a), wherein said nucleic acid molecule has at a position corresponding to position 201 of any one of SEQ ID NOs:1 to 77 an adenosine, corresponding to position 201 of any one of SEQ ID NOs:78 to 149 a guanosine, corresponding to position 201 of any one of SEQ ID NOs:150 to 219 a thymidine or corresponding to position 201 of any one of SEQ ID NOs:220 to 292 a cytidine; (c) a nucleic acid molecule identical to the nucleic acid molecule of (a) or (b) wherein each thymidine is replaced by uridine.

In this embodiment, the nucleic acid molecules contain the respective risk alleles associated with the presence or predisposition for androgenetic alopecia. Thus, preferably the nucleic acid molecule of the invention is for use in the diagnosis of androgenetic alopecia.

Further, the invention relates to a nucleic acid molecule selected from the group consisting of (a) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; (b) a nucleic acid molecule the complementary strand of which hybridises under stringent conditions to the nucleic acid molecule of (a), wherein said nucleic acid molecule has at a position corresponding to position 201 of any one of SEQ ID NOs: SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264 an adenosine, corresponding to position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215 a cytidine, corresponding to position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 a guanosine or corresponding to position 201 of any one of SEQ ID NOs: 6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292 a thymidine; and (c) a nucleic acid molecule identical to any one of the nucleic acid molecules of (a) or (b) wherein each thymidine is replaced by uridine.

In this embodiment, the nucleic acid molecules contain the respective non-risk alleles associated with the absence of androgenetic alopecia or the absence of a predisposition for androgenetic alopecia. Thus, the detection of these nucleic acid molecules is diagnostic of an absence of androgenetic alopecia or the absence of a predisposition for androgenetic alopecia. Preferably, this nucleic acid molecule of the invention is for use in the diagnosis of androgenetic alopecia.

Performing a genome-wide association analysis the present inventors found that 52 SNPs at the chromosome 20p11 locus showed association with androgenetic alopecia (P<0.05). These SNPs are located at position 201 of any one of SEQ ID NOs:1, 7, 23, 27, 30, 36, 39, 41, 42, 43, 51, 53, 60, 61, 62, 74, 77, 86, 92, 103, 104, 105, 106, 111, 116, 117, 118, 121, 132, 136, 141, 145, 153, 156, 157, 166, 175, 183, 198, 200, 208, 215, 255, 257, 258, 259, 270, 278, 286, 289, 290, and 292. The human genome has been analyzed in detail by the Human HapMap Project and the linkage disequilibrium between two SNPs in a genomic region is well known and the information is publicly available at www.hapmap.org. By combining the association data with the publicly available linkage disequilibrium data the inventors defined 240 additionally SNPs which are in linkage disequilibrium with at least one of the 52 associated SNPs. The risk alleles of the 240 SNPs are clearly defined by the risk alleles of the 52 associated SNPs and can therefore be used as a substitute of these. Any of these 292 genetic markers within the chromosomal region can thus be used by the skilled person to determine the presence or predisposition for androgenetic alopecia.

In a preferred embodiment the genetic marker is a SNP selected from the group consisting of the SNPs defined by any one of SEQ ID NOs:1, 7, 23, 27, 30, 36, 39, 41, 42, 43, 51, 53, 60, 61, 62, 74, 77, 86, 92, 103, 104, 105, 106, 111, 116, 117, 118, 121, 132, 136, 141, 145, 153, 156, 157, 166, 175, 183, 198, 200, 208, 215, 255, 257, 258, 259, 270, 278, 286, 289, 290 or 292. In another preferred embodiment of the method of the invention or the nucleic acid molecule of the invention, the nucleic acid molecule consists of at least 17 consecutive nucleotides from any of SEQ ID NOs:1, 7, 23, 27, 30, 36, 39, 41, 42, 43, 51, 53, 60, 61, 62, 74, 77, 86, 92, 103, 104, 105, 106, 111, 116, 117, 118, 121, 132, 136, 141, 145, 153, 156, 157, 166, 175, 183, 198, 200, 208, 215, 255, 257, 258, 259, 270, 278, 286, 289, 290 or 292.

In a more preferred embodiment, the genetic marker is a SNP selected from the group consisting of the SNPs defined by any one of SEQ ID NOs: 51, 105, 157, 175 or 198. In another more preferred embodiment of the method of the invention or the nucleic acid molecule of the invention, the nucleic acid molecule consists of at least 17 consecutive nucleotides from any of SEQ ID NOs: 51, 105, 157, 175 or 198.

In a preferred embodiment of the method of the invention or the nucleic acid molecule of the invention, the nucleic acid molecule is genomic DNA.

This preferred embodiment of the invention reflects the fact that usually the analysis would be carried out on the basis of genomic DNA from body fluid, cells or tissue isolated from the person under investigation.

The invention further relates to a nucleic acid molecule which is complementary to the nucleic acid molecule of the invention.

Complementary nucleic acid molecules are for example useful in the analysis of the genetic setup in the recited positions in hybridisation assays, such as in Southern blot analysis of DNA preparations, but also in PCRs or primer extension protocols respectively. Thus, for example, a 17mer exactly complementary either to a sequence having the non-risk allele or to a sequence having the risk allele associated with androgenetic alopecia may be used to differentiate between the polymorphic variants. This is because a nucleic acid molecule labelled with a detectable label not exactly complementary to the DNA in the analyzed sample will not give rise to a detectable signal, if appropriate hybridization and washing conditions are chosen. Preferably, these conditions are highly stringent conditions as described above.

In this regard, it is important to note that the nucleic acid molecule of the invention as well as the complementary nucleic acid molecule may be detectably labelled. Detectable labels include radioactive labels such as ³H, or ³²P or fluorescent labels. Labelling of nucleic acids is well understood in the art and described, for example, in Sambrook and Russel “Molecular Cloning, A Laboratory Manual”, Cold Spring Harbor Laboratory, N.Y. (2001).

Furthermore, as complementary nucleic acid molecules are also associated with androgenetic alopecia, they may, as described above, be directly analysed for the presence of the respective complementary nucleotide at position 201 of the nucleic acid molecule of the invention, which is indicative of the presence or predisposition for androgenetic alopecia.

In a further embodiment, the invention also relates to a vector comprising the nucleic acid molecule of the invention as described above.

The term “vector” in accordance with the present invention refers to a vector that is a plasmid, cosmid, virus, bacteriophage or another vector conventionally used e.g. in genetic engineering. Incorporation of the nucleic acid into a vector offers the possibility of introducing the nucleic acid molecule efficiently into the cells and preferably the DNA of a recipient. The recipient may be a single cell such as cell from a cell line. Such a measure renders it possible to express, when expression vectors are chosen, the respective nucleic acid molecule in the recipient. Thus, incorporation of the nucleic acid molecule into an expression vector opens up the way to a permanently elevated level of the encoded protein in any cell or a subset of selected cells of the recipient. Furthermore, chromosomal loci may be modified when using specific targeting vectors comprising a nucleic acid molecule to be replaced for the homologous chromosomal sequence of the host cell. Typical targeting vectors contain a positive selectable marker and the replacement construct, with two regions of homology to the target sequence located on either side. When transfected into cells, a double crossover event will replace the target sequences with the replacement construct sequences.

The nucleic acid molecule may be inserted into several commercially available vectors. Non-limiting examples include vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen), pCINeo (Promega), Okayama-Berg cDNA expression vector pcDV1 (Pharmacia), pRc/CMV, pcDNA1, pSPORT1 (GIBCO BRL), pGEMHE (Promega), pSVL and pMSG (Pharmacia, Uppsala, Sweden), pRSVcat (ATCC 37152), pSV2dhfr (ATCC 37146) or pBC12MI (ATCC 67109).

The polynucleotide of the present invention referred to above may also be inserted into vectors such that a translational fusion with another polynucleotide is generated. The other polynucleotide may encode a protein which may e.g. increase the solubility and/or facilitate the purification of the fusion protein. Non-limiting examples include pET32, pET41, pET43. The vectors may also contain an additional expressible polynucleotide coding for one or more chaperones to facilitate correct protein folding. Suitable bacterial expression hosts comprise e.g. strains derived from BL21 (such as BL21(DE3), BL21(DE3)PlysS, BL21(DE3)RIL, BL21(DE3)PRARE) or Rosetta®.

For vector modification techniques, see Sambrook and Russel (“Molecular Cloning, A Laboratory Manual”, Cold Spring Harbor Laboratory, N.Y. (2001)). Generally, vectors can contain one or more origin of replication (ori) and inheritance systems for cloning or expression, one or more markers for selection in the host, e.g., antibiotic resistance, and one or more expression cassettes.

The coding sequences inserted in the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in eukaryotic cells are well known to those skilled in the art. These elements comprise regulatory sequences ensuring the initiation of the transcription (e.g. translation initiation codon, promoters, enhancers, and/or insulators), internal ribosomal entry sites (IRES) (Owens Proc. Natl. Acad. Sci. USA 2001, 98: 1471) and optionally poly-A signals ensuring termination of transcription and stabilization of the transcript. Additional regulatory elements may include transcriptional as well as translational enhancers, and/or naturally-associated or heterologous promoter regions. Preferably, the nucleic acid molecule is operatively linked to such expression control sequences allowing expression in eukaryotic cells. The vector may further comprise nucleotide sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Possible examples for regulatory elements ensuring the initiation of transcription comprise the cytomegalovirus (CMV) promoter, SV40-promoter, RSV-promoter (Rous sarcome virus), the lacZ promoter, the gai10 promoter, human elongation factor 1a-promoter, CMV enhancer, CaM-kinase promoter, the Autographa californica multiple nuclear polyhedrosis virus (AcMNPV) polyhedral promoter or the SV40-enhancer. Examples for further regulatory elements in prokaryotes and eukaryotic cells comprise transcription termination signals, such as SV40-poly-A site or the tk-poly-A site or the SV40, lacZ and AcMNPV polyhedral polyadenylation signals, downstream of the polynucleotide. Moreover, elements such as origin of replication, drug resistance gene, regulators (as part of an inducible promoter) may also be included. Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter).

The co-transfection with a selectable marker such as dhfr, gpt, neomycin, hygromycin, ampicillin and kanamycin allows the identification and isolation of the transfected cells. The transfected nucleic acid can also be amplified to express large amounts of the encoded (poly)peptide. The DHFR (dihydrofolate reductase) marker is useful to develop cell lines that carry several hundred or even several thousand copies of the gene of interest. Another useful selection marker is the enzyme glutamine synthase (GS) (Murphy et al., Biochem J. 1991, 227:277; Bebbington et al., Bio/Technology 1992, 10:169). Using these markers, the mammalian cells are grown in selective medium and the cells with the highest resistance are selected. As indicated above, the expression vectors will preferably include at least one selectable marker. Such markers include dihydrofolate reductase, G418 or neomycin resistance for eukaryotic cell culture.

Specifically-designed vectors allow the shuttling of DNA between different hosts, such as bacteria-fungal cells or bacteria-animal cells (e.g. the Gateway® system available at Invitrogen).

The nucleic acid molecules of the invention as described herein above may be designed for direct introduction or for introduction via liposomes, phage vectors or viral vectors (e.g. adenoviral, retroviral) into the cell. Additionally, baculoviral systems or systems based on Vaccinia Virus or Semliki Forest Virus can be used as eukaryotic expression system for the nucleic acid molecules of the invention.

In a further embodiment, the present invention also relates to a host genetically engineered with the nucleic acid molecule of the invention or with the vector comprising the nucleic acid molecule of the invention as described above.

Preferably, introduction of the nucleic acid molecule of the invention or the vector comprising the nucleic acid molecule of the invention leads to integration of the nucleic acid molecule into the genome of the host. It is particularly envisaged that nucleic acid molecules of the invention may be introduced into the genome of the host by homologous recombination using techniques well known to the person skilled in the art. By integration of the nucleic acid molecules of the invention into the genome, regulatory effects of the particular SNPs, such as for example transcriptional regulation of neighbouring genes, may be investigated in suitable hosts.

The host cell may be any prokaryote or eukaryotic cell. Suitable prokaryotes/bacteria are those generally used for cloning like E. coli (e.g., E. coli strains BL21(DE3), HB101, DH5α, XL1 Blue, Y1090 and JM101), Salmonella typhimurium, Serratia marcescens, Pseudomonas putida, Pseudomonas fluorescens, Streptomyces lividans, Lactococcus lactis, Mycobacterium smegmatis or Bacillus subtilis. A suitable eukaryotic host cell may be an animal cell such as a mammalian cell, an amphibian cell, a fish cell, an insect cell such as Drosophila S2 and Spodoptera Sf9 cells, a fungal cell or a plant cell. Mammalian host cells that could be used include, human Hela, HEK293, H9, Bowes melanoma cells and Jurkat cells, mouse NIH3T3 and C127 cells, Cos 1, Cos 7 and CV1, quail QC1-3 cells, mouse L cells, Chinese hamster ovary (CHO) cells or non-human embryonic stem cells. Also within the scope of the present invention are primary mammalian cells such as the immortalised keratinocyte cell line HaCaT, primary keratinocytes, primary hair folllicle cells or mouse embryonic fibroblasts (MEF). Appropriate culture mediums and conditions for the above-described host cells are well known in the art.

A method for the production of a transgenic non-human animal, for example a transgenic mouse, comprises introduction of the aforementioned nucleic acid molecule or targeting vector into a germ cell, an embryonic cell, stem cell or an egg or a cell derived thereof. The non-human animal can be used in accordance with the invention in a method for identifying potential roles of the risk alleles of the nucleic acid molecule of the invention. Production of transgenic embryos and screening of those can be performed, e.g., as described by A. L. Joyner Ed., Gene Targeting, A Practical Approach (1993), Oxford University Press. The DNA of the embryonic membranes of embryos can be analyzed using, e.g., PCR amplification and sequencing or Southern blots with an appropriate probe; see supra. A general method for making transgenic non-human animals is described in the art, see for example WO 94/24274. For making transgenic non-human organisms (which include homologously targeted non-human animals), embryonal stem cells (ES cells) are preferred. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al., Nature 326:292-295 (1987)), the D3 line (Doetschman et al., J. Embryol. Exp. Morph. 87:27-45 (1985)), the CCE line (Robertson et al., Nature 323:445-448 (1986)), and the AK-7 line (Zhuang et al., Cell 77:875-884 (1994)). The success of generating a mouse line from ES cells bearing a specific targeted mutation depends on the pluripotence of the ES cells (i.e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant non-human females and are born, e.g. as chimeric mice. The resultant transgenic mice are backcrossed and screened for the presence of the correctly targeted transgene(s), usually by PCR or Southern blot analysis on tail biopsy DNA of offspring so as to identify transgenic mice.

The transgenic non-human animals may, for example, be transgenic mice, rats, hamsters, dogs, monkeys, rabbits, pigs, or cows. Preferably, said transgenic non-human animals are mice.

In another embodiment, the invention relates to a diagnostic composition for the detection of androgenetic alopecia or a predisposition for androgenetic alopecia comprising the nucleic acid molecules, the complementary nucleic acid molecules, the vector or the host cell of the invention.

The term “diagnostic composition” relates to compositions for diagnosing a predisposition of individual patients for androgenetic alopecia. The diagnostic composition of the invention comprises at least one of the nucleic acid molecules, the complementary nucleic acid molecules, the vector or the host cell recited above. Said composition may further comprise appropriate buffer(s), and enzymes such as reverse transcriptase, thermostable polymerases etc. The diagnostic composition of the invention may be used to test for the presence of the nucleic acid molecule of the invention using methods well known in the art. Such methods have been described above and are also described, e.g. in Sambrook and Russel “Molecular Cloning, A Laboratory Manual”, Cold Spring Harbor Laboratory, N.Y. (2001).

The invention also relates to a kit comprising the nucleic acid molecules of the invention, the complementary nucleic acid molecules, the vector containing the nucleic acid molecule or the host of the invention.

The various components of the kit may be packaged in one or more containers such as one or more vials. The vials may, in addition to the components, comprise preservatives or buffers for storage.

The figures show:

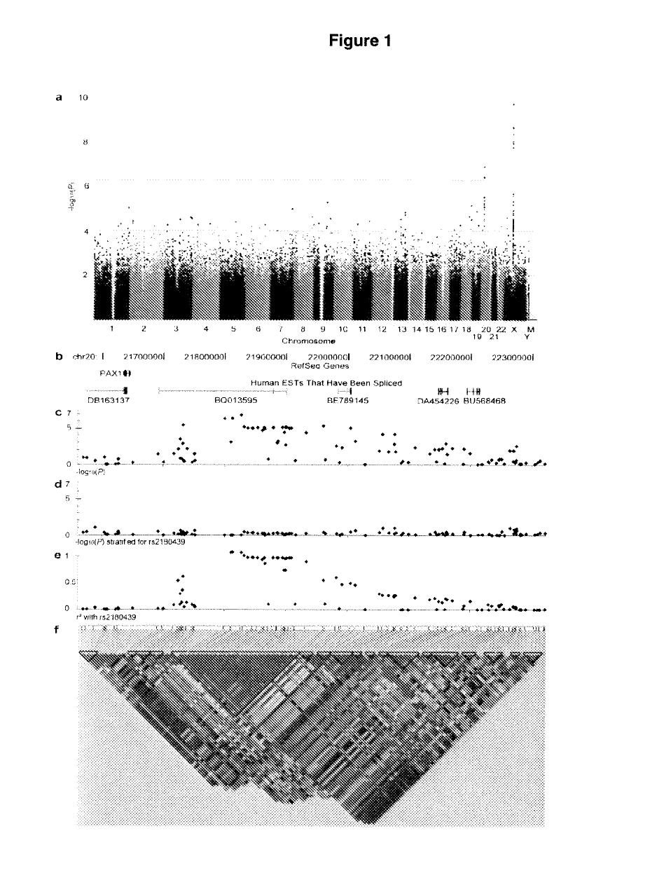

FIG. 1: Genome-wide scan for association with AGA and details of the region 21,571,171-22,321,597 on chromosome 20p11. (a) Negative log₁₀ of the Cochran-Armitage trend test P for the autosomes and of the allele frequency difference test P of chromosomes X, Y and the mitochondrion for quality-control-positive SNPs. Chromosomes are shown in alternating colours, with SNPs selected for the replication step highlighted in red. Genome-wide significance level is indicated by dashed red line. (b) Transcript information of the chromosome 20 locus (UCSC genome browser). (c) Negative log₁₀ association P are displayed for the GWAS (with rs2180439 indicated in red) and for an analysis stratified for rs2180439 (d). (e) The LD measured by r² between each SNP genotyped in the region and rs2180439 is shown. (f) LD displayed by GOLD heatmap and haplotype blocks indicated by black triangles were analyzed using Haploview software. High D′ is indicated in red, low D′ is indicated in blue.

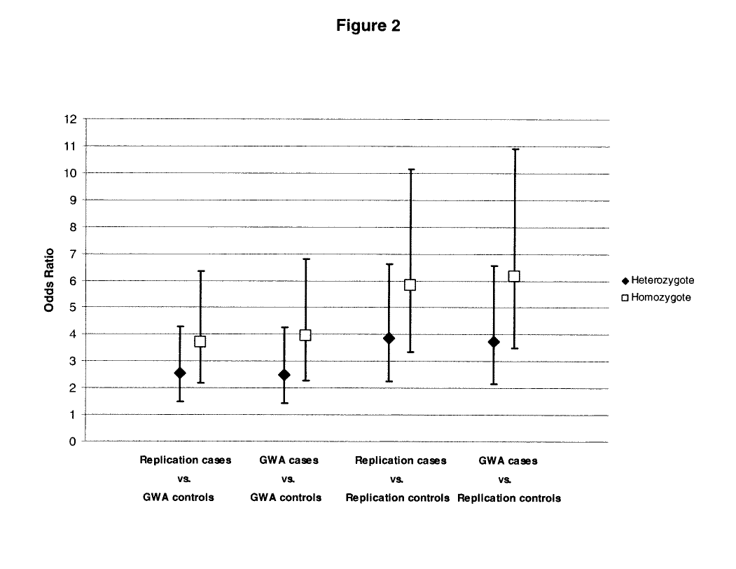

FIG. 2: Genotype odds ratios of rs2180439 with increasing phenotypic divergence between cases and controls. All cases had AGA that was representative of the most severely affected 10% of the distribution for the respective age class. Thereof, cases used in the GWAS were the most severely affecteds and cases used in the replication step were less extreme. Controls of the GWAS were population based where males with extreme AGA had been excluded. Controls of the replication step represented the least-affected 20% of the distribution for their age class. ORs for heterozygotes (C/T) are indicated by filled diamond, ORs for homozygotes (TIT) are indicated by open squares, 95% CIs are indicated by bars.

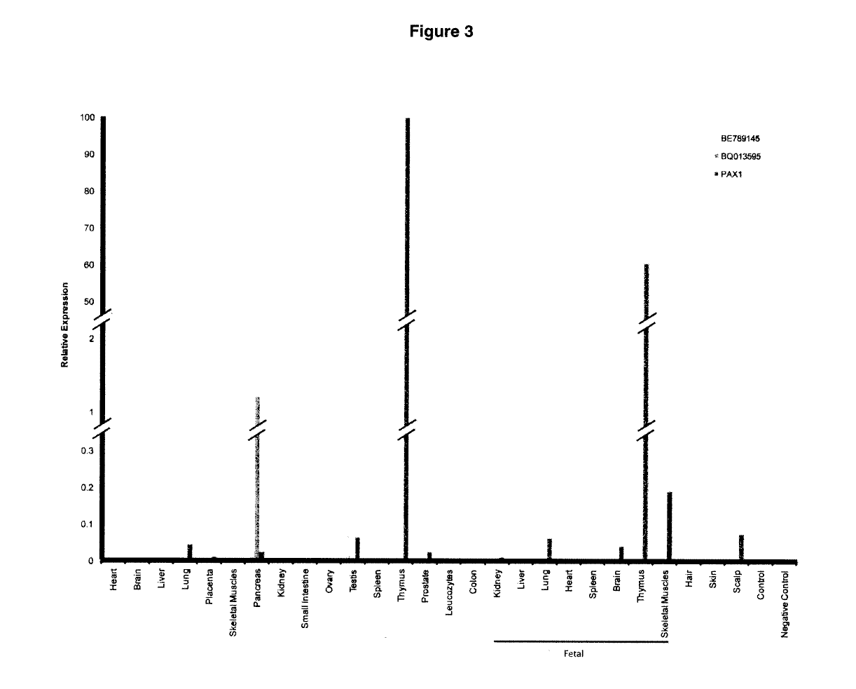

FIG. 3: Expression of BQ013595, BE789145, and PAX1 in human tissues. Expression levels were determined by real time PCR, normalized by cyclophilin and displayed relative to the strongest expression (PAX1 in adult thymus). PAX1 expression is detectable in scalp.

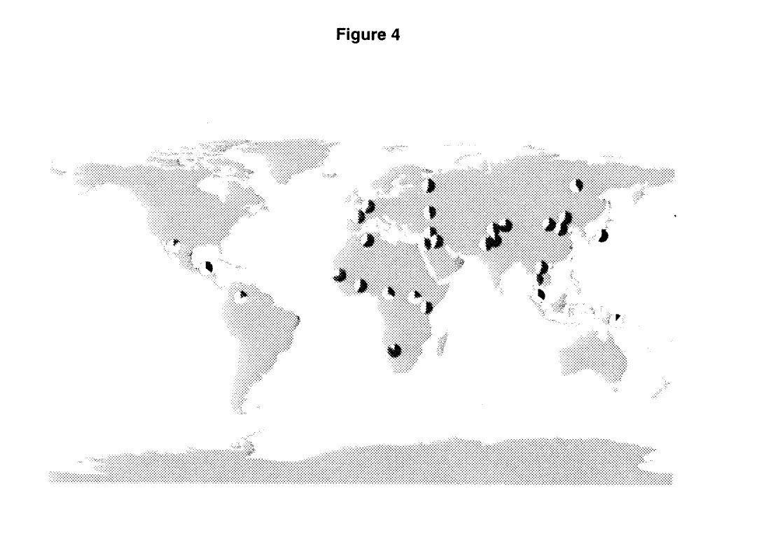

FIG. 4: Global distribution of the rs2180439 risk allele. Genotype data of the HGDP-CEPH Diversity Panel was extracted from the HGDP-CEPH Diversity Panel Database Supplement 2 at http://www.cephb.fr/hgdp-cephdb/. Worldwide distribution of the AGA risk allele T is indicated by the proportion of black shading in pie charts. Lower risk allele frequencies were observed in Oceanians and Native Americans

The examples illustrate the invention:

### EXAMPLE 1

### Materials and Methods

### German Sample

Families and unrelated AGA and non-AGA individuals were recruited through various sources including press reports, and advertisements in magazines, newspapers and placards. Blood samples were drawn from 754 affected males who were <30 years of age having AGA of grades IV-VII, based on the classification of Hamilton J B (1951), Ann N Y Acad Sci 53:708-728 and modified by Norwood O T (1975), South Med J 68:1359-1365, or <40 years having AGA of grades V-VII. Based on these criteria, the AGA sample was representative of the most severely affected 10% of the distribution for the respective age class (Hamilton J B (1951), Ann N Y Acad Sci 53:708-728; Norwood O T (1975), South Med J 68:1359-1365). Of the affecteds, the 296 unrelated individuals with the strongest hair loss (mean±SD age [in years] 33.3±4.9, Hamilton/Norwood grades: median VI, lower quartile V, upper quartile VII) were selected for GWAS and 319 independent unrelated affecteds (mean±SD age [in years] 33.4±5.1, Hamilton/Norwood grades: median V, lower quartile V, upper quartile VI) were selected for the replication step. 234 non-affected males were >60 years of age and without AGA (mean±SD age [in years] 67.6±5.9), representing the least affected 20% of the distribution for this age class. 491 of the 754 affecteds were part of 352 nuclear families comprising of parents with one to four affected sons. As controls, we investigated 383 individuals (mean±SD age [in years] 61.1±5.3) from the population-based Heinz Nixdorf RECALL cohort (Schmermund A, et al. (2002), Am Heart J 144:212-218) consisting of 182 male and 201 female probands (mean±SD age [in years] 61.1±5.3) who were randomly selected from the general population. Of 129 male controls, hair status information was available: 16 individuals with grade I (Hamilton/Norwood classification), 21 individuals with grade II, one individual with grade IIa, eight individuals with grade III, 21 individuals with grade III vertex, four individuals with grade IIIa, ten individuals with grade IV, one individual with grade IVa, eleven individuals with grade V, 27 individuals with grade VI, and nine individuals with grade VII. Thirty six males with AGA grade VI and VII, respectively, were excluded from GWAS to increase phenotypic stratification between cases and controls. Females were included in the GWAS control sample as they were considered to show the representative AGA risk allele frequencies for population controls. The study was approved by the Ethics Committees of the Universities of Bonn, Düsseldorf and Essen, and informed consent was obtained from all participating individuals. All participants were of German descent.

### Australian Sample

As part of a population based twin study (Nyholt D R, et al. (2003), J Invest Dermatol 121:1561-1564), hair status was documented in 291 unrelated male individuals for whom DNA was available. Measures of hair loss were obtained in the course of an extensive semi-structured telephone interview with respondent booklet, designed to assess physical, psychological and social manifestations of alcoholism and related disorders, conducted with 6265 twins born 1964-71 from the volunteer based Australian Twin Registry. All males (45% of the sample) were asked to rate their degree of hair loss, if any, using the Hamilton-Norwood Baldness scale (Hamilton J B (1951), Ann N Y Acad Sci 53:708-728; Norwood O T (1975), South Med J 68:1359-1365), which was printed in the respondent booklet. This data collection scheme was validated in a study by Ellis J A, et al. (1998), J Invest Dermatol 110:849-853. Individuals with hair loss Hamilton/Norwood grade≧IIa were classified as AGA affecteds and individuals with Hamilton/Norwood grade I were classified as controls. The study was approved by the Human Research Ethics Committee of the Queensland Institute of Medical Research, and informed consent was obtained from all participating individuals.

### DNA Extraction

EDTA anticoagulated venous blood samples were collected from all individuals. Lymphocyte DNA from German AGA affecteds, non-affecteds, families, and Australian samples was isolated by salting out with saturated NaCl solution (Miller S A, et al. (1988), Nucleic Acids Res 16:1215) and DNA from German population-based controls was isolated using a Chemagic Magnetic Separation Module I (Chemagen, Baesweiler, Germany) according to the manufacturer recommendations. Genomic DNAs were diluted to working concentrations of 50 ng/μl for genome-wide genotyping and 2.5 ng/μl for the replication step.

### Genotypinq

Genome-wide genotyping of 317,503 SNPs in 150 AGA affecteds using the Illumina HumanHap300 BeadChip and of 561,466 SNPs in 146 affecteds and 383 population based controls using the Illumina HumanHap550 BeadChip was conducted according to the Infinium II protocol from Illumina (Illumina, San Diego, USA). A DNA sample was deemed to have failed if it generated genotypes at fewer than 95% of loci of the respective BeadChip. A SNP was deemed to have failed if fewer than 95% of case or control samples generated a genotype at the locus. SNPs were excluded with a minor allele frequency (MAF)<0.01 in controls and a Hardy-Weinberg-Equilibrium (HWE) P<10⁻⁷ in controls. 531,695 SNPs passed quality criteria and were used for association analysis.

For the replication study, the best SNPs were selected based on the P values of the Cochran-Armitage trend test for the autosomes and the pseudoautosomal region and based on the allele frequency difference test for chromosomes X, Y, and the mitochondrion. To maximize efficiency in the replication step, four tagging SNPs of chromosome 20p11 were selected using the pairwise tagging algorithm of Haploview software (Barrett J C, et al. (2005), Bioinformatics 21:263-265) (parameters: r²≧0.8 and LOD≧3) and CEU HapMap data (Frazer K A, et al. (2007), Nature 449:851-861). The genome-wide best SNP, rs19998076 (Supplementary Table 1), was forced to be included and rs2180439 which was in perfect LD with rs1998076 was added for additional confidence in the most significant SNP. The genome-wide next best 30 SNPs excluding the AR locus (chromosome X 65.5-67.2 Mb, build 36) were selected for replication analysis. Visual inspection of genotype clusters of selected SNPs revealed for rs4976846 a shifted genotype cluster for a subset of samples and the SNP was excluded from the genome-wide data set. Of the 34 selected SNPs four had to be excluded: rs11655206 and rs4805229 could not be included in the two plexes for MassArray genotyping, rs13079866 was a genotyping failure, and rs1139488 showed fuzzy genotype clusters, significant deviation from HWE, and 19 Mendel errors, respectively, in the family data.

Genotyping for the replication step in the German and Australian samples was performed using the MassArray system on a Sequenom Compact MALDI-TOF device (Sequenom Inc., San Diego, Calif., USA). Primer sequences and PCR/assay conditions can be obtained from the authors upon request. SNP call rates were >95.0% and samples with call rates <95% were excluded from the analysis.

### Statistical Analysis

Genome-wide association analysis at single marker level was performed by Cochran-Armitage trend test for the autosomes and the pseudoautosomal region and by the allele frequency difference test for chromosomes X, Y, and the mitochondrion. P<5×10⁻⁷ were assumed to be genome-wide significant as suggested by The Wellcome Trust Case Control Consortium (2007), Nature 447:661-678. Haplotype and family based analysis of the chromosome 20 locus was conducted with FAMHAP software (Becker T, et al. (2005), Ann Hum Genet 69:747-756). For case-control haplotype analysis, P values were calculated from the χ² distribution with n−1 degrees of freedom of the respective likelihood-ratio test (n=number of different haplotypes). The family data were analyzed with the permutation-based association test for nuclear families (Zhao H, et al. (2000), Am J Hum Genet 67:936-946; Knapp M, Becker T (2003), Hum Hered 56:2-9). For each marker (single locust analysis) and each marker combination (haplotype analysis), we used 10¹⁰ permutation replicates. As rs2180439 was the most associated marker in the German combined sample, we carried out further statistical analysis based on that marker in the compound data set. We used logistic regression as suggested by Cordell H J, Clayton D G (2002), Am J Hum Genet 70:124-141. First, we tested for deviation from a multiplicative model on the OR scale by comparing a 2-degree of freedom (DF) model with parameter β₁ for a multiplicative SNP-effect and parameter β₂ for a dominance effect versus a 1-DF model with just β₁. Secondly, we tested for independent effects of the remaining four SNPs of the region. Therefore, we tested a 3-DF model with parameters β₁, β₂ and β₃ (parameter for multiplicative effect of additional SNP) versus the 2-DF model for rs2180439 (β₁, β₂). If this test was significant, we further tested for additional dominance at the new SNP (parameter β₄) by comparing the 4-DF model to the 3-DF model.

In order to test for interaction with the X chromosome, we considered the initial GWAS case sample (n=296) versus the male controls (n=146). For this data set, the model with β₁, β₃ and β₄ had a P value of 5.89×10⁻⁶. Introduction of the X-chromosomal SNP rs1041668 improved the model-fit significantly (P=1.32×10⁻⁸), resulting in a 4-DF model with P=1.17×10⁻¹³. On top of this model, neither the interaction terms with rs2180439 (P=0.975) nor with rs6113491 (P=0.984) yielded significant improvement of the model fit.

### Expression Analysis

To test for tissue specific expression of BQ013595, BE789145 and PAX1, we used the Human Multiple-Tissue cDNA (MTC) Panels I, II and the Human Fetal MTC Panel (Takara Bio Europe/Clontech, Saint-Germain-en-Laye, France). In addition, total RNA from hair follicles, skin and scalp was extracted (RNeasy Micro Kit, Qiagen). Quality and quantity of the RNA was analyzed on a NanoDrop ND-1000 spectrophotometer (Peqlab Biotechnologie, Erlangen, Germany) and reverse transcription was performed using SuperScript First Strand Synthesis System for RT-PCR (Invitrogen, Karlsruhe, Germany) with oligo dT primers. cDNAs were adjusted in an initial real-time PCR experiment using the TaqMan Endogenous Controls Human Cyc (Cyclophilin, 4326316E; Applied Biosystems, Darmstadt, Germany) in which 5 μl of the MTC-cDNAs were set as reference. Relative quantifications in real-time experiments were performed in 384-well plates with the adjusted amounts of cDNAs per well in a total volume of 20 μl on the ABI Prism 7900HT Fast Real-Time PCR System (Applied Biosystems, Darmstadt, Germany) using Custom TaqMan Gene Expression Assays for BQ013595 (forward primer AGACATACTTTTCTATTCACCTTCCAATGATG, reverse primer GGTTCTGACCCTTATTATTGGAACTCA, probe AAGTCCCAACAAACTCCA), BE789145 (forward primer ACCTGACATCAACGTACAAAGATTCA, reverse primer GCGGCTTCTCCTCTGCAT, probe CATGGCCACAAGAGTTA), and PAX1 (forward primer GGCTAGAGAAACCTGCCTTAGAG, reverse primer CGCCCACGGCAGAGA, probe ACACTCAGTCGGCCTCC). Each sample was normalized by TaqMan Endogenous Controls Human Cyc, and CT values were transformed by 100,000,000/(2^{CT}). Values were calculated relative to the expression of PAX1 in thymus which was set as 100%.

### EXAMPLE 2

### Genome-Wide Association Study

We conducted a genome-wide association study (GWAS) to identify predisposing genetic factors in AGA. The analysis was performed on 296 males with early-onset AGA (Hamilton/Norwood hair loss grades: median VI, lower quartile V, upper quartile VII) and 383 population based controls (201 females and 182 males), all of German descent, were genotyped with Illumina BeadChips (HumanHap300/HumanHap550). For the analysis, 36 male controls with strong AGA were excluded resulting in 347 controls. Following quality control, we performed association analysis on 531,695 single nucleotide polymorphisms (SNPs) with a minor allele frequency (MAF) of >1% in controls. The SNP call rate was 99.78%.

We found highly significant association in a 1.7 Mb region flanking the previously implicated AR locus (FIG. 1). The best SNP (rs1998076, P=1.3×10⁻⁷) located outside the AR locus was on chromosome 20, a locus which was supported by 17 additional SNPs with P<10⁻⁵.

TABLE 2
Genome-wide association results for AGA (excluding AR locus).

| SNP | Chr. | Position [bp]^{a} | MAF cases | MAF controls | P | OR (95% CI) |
|---|---|---|---|---|---|---|
| rs1998076 | 20 | 21,828,045 | 0.282 (A) | 0.427 (A) | 1.30 × 10⁻⁷ | 1.90 (1.50-2.41) |
| rs6075852 | 20 | 21,814,151 | 0.292 (A) | 0.429 (A) | 3.50 × 10⁻⁷ | 1.83 (1.45-2.31) |
| rs2180439 | 20 | 21,801,100 | 0.292 (C) | 0.429 (C) | 3.85 × 10⁻⁷ | 1.82 (1.45-2.30) |
| rs6137444 | 20 | 21,733,639 | 0.264 (C) | 0.383 (C) | 3.11 × 10⁻⁶ | 1.74 (1.37-2.21) |
| rs201571 | 20 | 21,961,514 | 0.289 (C) | 0.411 (C) | 4.31 × 10⁻⁶ | 1.72 (1.36-2.17) |
| rs10992241 | 9 | 93,931,599 | 0.156 (C) | 0.262 (C) | 5.31 × 10⁻⁶ | 1.92 (1.44-2.54) |
| rs6137473 | 20 | 21,832,693 | 0.321 (G) | 0.445 (G) | 5.58 × 10⁻⁶ | 1.70 (1.35-2.13) |
| rs6047768 | 20 | 21,901,649 | 0.331 (G) | 0.457 (G) | 6.09 × 10⁻⁶ | 1.70 (1.35-2.13) |
| rs2207878 | 20 | 21,899,847 | 0.331 (G) | 0.457 (G) | 6.19 × 10⁻⁶ | 1.70 (1.35-2.13) |
| rs6035995 | 20 | 21,879,683 | 0.331 (G) | 0.455 (G) | 6.97 × 10⁻⁶ | 1.69 (1.35-2.13) |
| rs6113424 | 20 | 21,854,780 | 0.331 (G) | 0.455 (G) | 7.05 × 10⁻⁶ | 1.69 (1.35-2.12) |
| rs2024885 | 20 | 21,865,393 | 0.331 (A) | 0.455 (A) | 7.05 × 10⁻⁶ | 1.69 (1.35-2.12) |
| rs201543 | 20 | 21,894,928 | 0.331 (A) | 0.455 (A) | 7.05 × 10⁻⁶ | 1.69 (1.35-2.12) |
| rs17817969 | 18 | 70,473,226 | 0.223 (T) | 0.117 (T) | 7.16 × 10⁻⁶ | 2.17 (1.51-3.11) |
| rs6047769 | 20 | 21,906,049 | 0.333 (A) | 0.457 (A) | 8.47 × 10⁻⁶ | 1.68 (1.34-2.12) |
| rs1884592 | 20 | 21,838,690 | 0.331 (C) | 0.454 (C) | 8.58 × 10⁻⁶ | 1.68 (1.34-2.11) |
| rs6137476 | 20 | 21,845,656 | 0.331 (A) | 0.454 (A) | 8.58 × 10⁻⁶ | 1.68 (1.34-2.11) |
| rs6047731 | 20 | 21,847,141 | 0.331 (G) | 0.454 (G) | 8.58 × 10⁻⁶ | 1.68 (1.34-2.11) |
| rs6113491 | 20 | 22,005,415 | 0.359 (C) | 0.483 (C) | 8.63 × 10⁻⁶ | 1.66 (1.33-2.08) |
| rs1555264 | 20 | 21,909,410 | 0.332 (G) | 0.455 (G) | 8.64 × 10⁻⁶ | 1.68 (1.34-2.11) |
| rs4658627 | 1 | 242,577,799 | 0.172 (A) | 0.308 (A) | 9.60 × 10⁻⁶ | 2.14 (1.51-3.02) |
| rs11975187 | 7 | 153,863,909 | 0.490 (A) | 0.359 (G) | 1.02 × 10⁻⁵ | 1.86 (1.41-2.46) |
| rs4805229 | 19 | 33,692,043 | 0.380 (T) | 0.268 (T) | 1.34 × 10⁻⁵ | 1.67 (1.32-2.12) |
| rs9300398 | 13 | 96,690,316 | 0.363 (T) | 0.252 (T) | 1.46 × 10⁻⁵ | 1.69 (1.33-2.15) |
| rs4771987 | 13 | 96,699,391 | 0.429 (G) | 0.315 (G) | 1.88 × 10⁻⁵ | 1.63 (1.30-2.05) |
| rs3786343 | 18 | 59,798,875 | 0.458 (T) | 0.340 (T) | 2.27 × 10⁻⁵ | 1.64 (1.31-2.05) |
| rs6986303 | 8 | 134,547,711 | 0.309 (A) | 0.205 (A) | 2.33 × 10⁻⁵ | 1.74 (1.35-2.24) |
| rs9856948 | 3 | 189,240,095 | 0.419 (C) | 0.304 (C) | 2.51 × 10⁻⁵ | 1.65 (1.31-2.08) |
| rs1139488 | 9 | 115,193,721 | 0.306 (C) | 0.418 (C) | 2.77 × 10⁻⁵ | 1.63 (1.29-2.05) |
| rs13079866 | 3 | 196,641,541 | 0.061 (G) | 0.131 (G) | 2.84 × 10⁻⁵ | 2.33 (1.56-3.49) |
| rs7319347 | 13 | 66,977,210 | 0.072 (C) | 0.173 (C) | 3.00 × 10⁻⁵ | 2.70 (1.66-4.38) |
| rs17818946 | 18 | 70,519,734 | 0.170 (G) | 0.082 (G) | 3.06 × 10⁻⁵ | 2.30 (1.52-3.47) |
| rs6804475 | 3 | 113,152,321 | 0.246 (A) | 0.352 (A) | 3.22 × 10⁻⁵ | 1.66 (1.30-2.12) |
| rs500629 | 11 | 113,550,770 | 0.346 (G) | 0.241 (G) | 3.62 × 10⁻⁵ | 1.66 (1.30-2.12) |
| rs10495747 | 2 | 24,368,500 | 0.149 (C) | 0.075 (C) | 3.78 × 10⁻⁵ | 2.16 (1.50-3.10) |
| rs1464766 | 5 | 111,641,241 | 0.243 (A) | 0.372 (A) | 4.14 × 10⁻⁵ | 1.84 (1.35-2.51) |
| rs2551182 | 2 | 24,293,921 | 0.128 (T) | 0.061 (T) | 4.75 × 10⁻⁵ | 2.29 (1.54-3.39) |
| rs300639 | 9 | 32,776,553 | 0.067 (T) | 0.021 (T) | 4.83 × 10⁻⁵ | 3.45 (1.85-6.42) |
| rs10001582 | 4 | 124,578,020 | 0.364 (A) | 0.478 (A) | 4.91 × 10⁻⁵ | 1.60 (1.28-2.01) |
| rs11584662 | 1 | 184,847,447 | 0.417 (T) | 0.467 (G) | 4.92 × 10⁻⁵ | 1.60 (1.28-1.99) |
| rs1009070 | 11 | 29,484,237 | 0.106 (G) | 0.038 (G) | 5.20 × 10⁻⁵ | 3.05 (1.78-5.24) |
| rs880013 | 9 | 1,425,496 | 0.474 (T) | 0.415 (C) | 5.28 × 10⁻⁵ | 1.56 (1.25-1.95) |
| rs4896028 | 6 | 134,580,872 | 0.309 (G) | 0.209 (G) | 5.35 × 10⁻⁵ | 1.69 (1.32-2.18) |
| rs11725633 | 4 | 37,868,359 | 0.368 (T) | 0.483 (T) | 5.57 × 10⁻⁵ | 1.60 (1.28-2.00) |
| rs11655206 | 17 | 72,501,171 | 0.497 (T) | 0.361 (T) | 5.60 × 10⁻⁵ | 1.74 (1.32-2.30) |
| rs6550859 | 3 | 24,385,419 | 0.390 (G) | 0.468 (A) | 5.72 × 10⁻⁵ | 1.77 (1.34-2.34) |
| rs3786939 | 19 | 44,888,022 | 0.301 (A) | 0.184 (A) | 5.79 × 10⁻⁵ | 1.91 (1.39-2.61) |

(^{a}Build 36)

Three SNPs at this locus reached genome-wide significance based on criteria suggested elsewhere (Consortium TWTCC (2007), Nature 447:661-678).

### EXAMPLE 3

### Replication Study

To replicate the association findings, four tagging SNPs of the chromosome 20 region and the next best 29 SNPs (excluding the AR locus) were selected. In addition, rs2180439, which was in perfect LD with rs1998076, was included for the purpose of providing additional confidence in the most significant SNP. Selected markers were analyzed in an independent set of 319 affected and 234 unaffected individuals (males >60 years of age without AGA). The association with all five SNPs of the chromosome 20 locus was replicated (P=8.13×10⁻¹⁰ for rs6113491).

TABLE 3
Replication analysis in 319 German cases and 234 German controls

| SNP | Chr. | Position [bp]^{a} | MAF cases | MAF controls | P | OR (95% CI) |
|---|---|---|---|---|---|---|
| rs6113491 | 20 | 22,005,415 | 0.364 (C) | 0.447 (A) | 8.13 × 10⁻¹⁰ | 2.17 (1.7-2.77) |
| rs2180439 | 20 | 21,801,100 | 0.303 (C) | 0.485 (C) | 1.37 × 10⁻⁰⁹ | 2.17 (1.7-2.78) |
| rs1998076 | 20 | 21,828,045 | 0.301 (A) | 0.479 (A) | 3.69 × 10⁻⁰⁹ | 2.13 (1.66-2.73) |
| rs201571 | 20 | 21,961,514 | 0.313 (C) | 0.483 (C) | 2.21 × 10⁻⁰⁸ | 2.05 (1.6-2.62) |
| rs6137444 | 20 | 21,733,639 | 0.277 (C) | 0.404 (C) | 1.57 × 10⁻⁰⁵ | 1.76 (1.37-2.27) |
| rs10992241 | 9 | 91,971,333 | 0.289 (C) | 0.201 (C) | 8.68 × 10⁻⁰⁴ | 1.62 (1.22-2.15) |
| rs11725633 | 4 | 38,014,530 | 0.422 (T) | 0.479 (T) | 0.058 | 1.26 (0.99-1.6) |
| rs4771987 | 13 | 96,699,391 | 0.417 (G) | 0.363 (G) | 0.075 | 1.25 (0.98-1.6) |
| rs6804475 | 3 | 113,152,321 | 0.292 (A) | 0.340 (A) | 0.081 | 1.25 (0.97-1.62) |
| rs3786939 | 19 | 44,888,022 | 0.202 (A) | 0.162 (A) | 0.085 | 1.31 (0.96-1.79) |
| rs880013 | 9 | 1,425,496 | 0.431 (C) | 0.479 (C) | 0.123 | 1.21 (0.95-1.54) |
| rs500629 | 11 | 113,550,770 | 0.307 (G) | 0.276 (G) | 0.249 | 1.17 (0.9-1.52) |
| rs9300398 | 13 | 96,690,316 | 0.335 (T) | 0.303 (T) | 0.252 | 1.16 (0.9-1.5) |
| rs1009070 | 11 | 29,484,237 | 0.062 (G) | 0.076 (G) | 0.35 | 1.25 (0.78-2) |
| rs4896028 | 6 | 134,580,872 | 0.268 (G) | 0.244 (G) | 0.368 | 1.14 (0.86-1.5) |
| rs10495747 | 2 | 24,426,647 | 0.130 (C) | 0.115 (C) | 0.458 | 1.15 (0.8-1.65) |
| rs17817969 | 18 | 70,473,226 | 0.147 (T) | 0.132 (T) | 0.483 | 1.13 (0.8-1.6) |
| rs6986303 | 8 | 134,547,711 | 0.288 (A) | 0.269 (A) | 0.495 | 1.1 (0.84-1.43) |
| rs10001582 | 4 | 124,716,175 | 0.429 (A) | 0.409 (A) | 0.51 | 1.09 (0.85-1.38) |
| rs3786343 | 18 | 59,798,875 | 0.395 (T) | 0.376 (T) | 0.527 | 1.08 (0.85-1.38) |
| rs1464766 | 5 | 111,641,241 | 0.324 (A) | 0.342 (A) | 0.545 | 1.08 (0.84-1.39) |
| rs11975187 | 7 | 153,670,624 | 0.346 (G) | 0.363 (G) | 0.559 | 1.08 (0.84-1.38) |
| rs11584662 | 1 | 183,312,481 | 0.436 (T) | 0.453 (T) | 0.573 | 1.07 (0.84-1.36) |
| rs17818946 | 18 | 70,519,734 | 0.108 (G) | 0.098 (G) | 0.588 | 1.11 (0.75-1.65) |
| rs6550859 | 3 | 24,385,419 | 0.498 (A) | 0.483 (A) | 0.608 | 1.06 (0.84-1.35) |
| rs9856948 | 3 | 189,240,103 | 0.350 (C) | 0.340 (C) | 0.735 | 1.04 (0.81-1.34) |
| rs2551182 | 2 | 24,352,068 | 0.099 (T) | 0.096 (T) | 0.886 | 1.03 (0.69-1.54) |
| rs7319347 | 13 | 66,977,210 | 0.160 (C) | 0.162 (C) | 0.91 | 1.02 (0.74-1.41) |
| rs4658627 | 1 | 240,837,217 | 0.270 (A) | 0.274 (A) | 0.911 | 1.02 (0.78-1.33) |
| rs300639 | 9 | 32,776,553 | 0.023 (T) | 0.023 (T) | 1 | 1 (0.44-2.27) |

(^{a}Build 36)

In addition, rs10992241 on chromosome 9 showed significance. However, the putative risk allele (C) in the replication study differed from that in the GWAS (T), and thus rs10992241 was not considered to be truly associated. After combining the samples from the genome-wide and replication analyses, rs2180439 showed the highest significance (P=2.67×10⁻¹⁵, Table 4). Haplotype analysis did not significantly improve the association findings.

TABLE 4
Association between the chromosome 20 locus and AGA in the German sample

| | | | | MAF | | Genotypes | | | |
|---|---|---|---|---|---|---|---|---|---|
| SNP (position)^{a} | Sample | Cases^{b} | Controls^{c} | Cases^{b} | Controls^{c} | Cases^{b} | Controls^{c} | P | OR (95% CI)^{d} |
| rs6137444 | GWAS | 296 | 347 | 0.264 (C) | 0.383 (C) | 14/128/154 | 49/168/130 | 3.11 × 10⁻⁶ | 1.74 (1.37-2.21) |
| (21,733,639 bp) | Replication | 319 | 234 | 0.277 (C) | 0.404 (C) | 21/135/163 | 45/99/90 | 1.57 × 10⁻⁵ | 1.76 (1.37-2.27) |
| | Combined^{e} | 605 | 579 | 0.269 (C) | 0.391 (C) | 35/255/315 | 93/267/219 | 2.20 × 10⁻¹⁰ | |
| rs2180439 | GWAS | 296 | 347 | 0.292 (C) | 0.429 (C) | 21/131/144 | 66/166/115 | 3.85 × 10⁻⁷ | 1.82 (1.45-2.30) |
| (21,801,100 bp) | Replication | 319 | 234 | 0.303 (C) | 0.485 (C) | 23/147/149 | 62/103/69 | 1.37 × 10⁻⁹ | 2.17 (1.70-2.78) |
| | Combined^{e} | 605 | 579 | 0.293 (C) | 0.452 (C) | 43/268/294 | 127/269/183 | 2.67 × 10⁻¹⁵ | |
| rs1998076 | GWAS | 296 | 347 | 0.282 (A) | 0.427 (A) | 20/120/144 | 65/163/115 | 1.30 × 10⁻⁷ | 1.9 (1.50-2.41) |
| (21,828,045 bp) | Replication | 319 | 234 | 0.301 (A) | 0.479 (A) | 23/146/150 | 61/102/71 | 3.69 × 10⁻⁹ | 2.13 (1.66-2.73) |
| | Combined^{e} | 605 | 579 | 0.292 (A) | 0.448 (A) | 43/267/295 | 126/267/186 | 7.73 × 10⁻¹⁵ | |
| rs201571 | GWAS | 296 | 347 | 0.289 (C) | 0.411 (C) | 17/137/142 | 61/163/123 | 4.31 × 10⁻⁶ | 1.72 (1.36-2.17) |
| (21,961,514 bp) | Replication | 319 | 234 | 0.314 (C) | 0.483 (C) | 30/140/149 | 58/110/66 | 2.21 × 10⁻⁸ | 2.05 (1.60-2.62) |
| | Combined^{e} | 605 | 579 | 0.298 (C) | 0.44 (C) | 46/269/290 | 119/272/188 | 1.21 × 10⁻¹² | |
| rs6113491 | GWAS | 296 | 347 | 0.359 (C) | 0.483 (C) | 29/154/112 | 88/159/100 | 8.63 × 10⁻⁶ | 1.66 (1.33-2.08) |
| (22,005,415 bp) | Replication | 319 | 234 | 0.364 (C) | 0.447 (A) | 38/156/125 | 77/105/52 | 8.13 × 10⁻¹⁰ | 2.17 (1.70-2.77) |
| | Combined^{e} | 605 | 579 | 0.359 (C) | 0.488 (A) | 66/302/237 | 165/263/151 | 1.13 × 10⁻¹³ | |

^{a}NCBI build 36.
^{b}GWAS cases were the most extremely affected.
^{c}GWAS controls are population based, the most extreme AGA affected having been excluded; replication controls are males >60 years of age without AGA and representing the least affected 20%.
^{d}ORs were not calculated for the combined samples since the composition of the two control samples differed as regards the exclusion of AGA cases.
^{e}Twelve GWAS samples were excluded in the combined analysis due to a call rate <95% for the reduced SNP set.

TABLE 5
Haplotype association analysis in the combined German data set

| Haplotype | | Frequency | Frequency | | | | | | |
|---|---|---|---|---|---|---|---|---|---|
| number | Haplotype^{a} | cases | controls | P | rs6137444 | rs2180439 | rs1998076 | rs201571 | rs6113491 |
| H1 | | | | 6.50 × 10⁻¹⁴ | X | X | | | |
| H1 | C-C | 0.233 | 0.365 | 1.99 × 10⁻¹² | | | | | |
| H1 | T-C | 0.060 | 0.087 | 1.20 × 10⁻⁰² | | | | | |
| H1 | C-T | 0.036 | 0.026 | 1.81 × 10⁻⁰¹ | | | | | |
| H1 | T-T | 0.671 | 0.522 | 1.19 × 10⁻¹³ | | | | | |
| H2 | | | | 1.74 × 10⁻¹³ | X | | X | | |
| H2 | C-A | 0.232 | 0.361 | 4.79 × 10⁻¹² | | | | | |
| H2 | T-A | 0.060 | 0.087 | 1.21 × 10⁻⁰² | | | | | |
| H2 | C-G | 0.037 | 0.030 | 3.47 × 10⁻⁰¹ | | | | | |
| H2 | T-G | 0.671 | 0.522 | 1.20 × 10⁻¹³ | | | | | |
| H3 | | | | 1.58 × 10⁻¹³ | X | | | X | |
| H3 | C-C | 0.179 | 0.289 | 2.61 × 10⁻¹⁰ | | | | | |
| H3 | T-C | 0.119 | 0.152 | 2.17 × 10⁻⁰² | | | | | |
| H3 | C-T | 0.090 | 0.102 | 2.90 × 10⁻⁰¹ | | | | | |
| H3 | T--T | 0.612 | 0.457 | 4.23 × 10⁻¹⁴ | | | | | |
| H4 | | | | 1.65 × 10⁻¹⁴ | X | | | | X |
| H4 | C-C | 0.190 | 0.312 | 5.13 × 10⁻¹² | | | | | |
| H4 | T-C | 0.169 | 0.200 | 5.47 × 10⁻⁰² | | | | | |
| H4 | C-A | 0.079 | 0.079 | 9.85 × 10⁻⁰¹ | | | | | |
| H4 | T-A | 0.562 | 0.409 | 8.92 × 10⁻¹⁴ | | | | | |
| H5 | C-A | 0.292 | 0.450 | 1.95 × 10⁻¹⁵ | | X | X | | |
| H5 | T-G | 0.708 | 0.550 | 1.95 × 10⁻¹⁵ | | | | | |
| H6 | | | | 7.02 × 10⁻¹⁵ | | X | | X | |
| H6 | C-C | 0.236 | 0.374 | 2.32 × 10⁻¹³ | | | | | |
| H6 | T-C | 0.063 | 0.066 | 7.23 × 10⁻⁰¹ | | | | | |
| H6 | C-T | 0.057 | 0.078 | 4.54 × 10⁻⁰² | | | | | |
| H6 | T-T | 0.645 | 0.482 | 1.45 × 10⁻¹⁵ | | | | | |
| H7 | | | | 1.01 × 10⁻¹⁵ | | X | | | X |
| H7 | C-C | 0.246 | 0.397 | 2.75 × 10⁻¹⁵ | | | | | |
| H7 | T-C | 0.113 | 0.115 | 8.82 × 10⁻⁰¹ | | | | | |
| H7 | C-A | 0.047 | 0.055 | 4.00 × 10⁻⁰¹ | | | | | |
| H7 | T-A | 0.594 | 0.433 | 4.61 × 10⁻¹⁵ | | | | | |
| H8 | | | | 1.44 × 10⁻¹⁴ | | | X | X | |
| H8 | A-C | 0.235 | 0.372 | 3.09 × 10⁻¹³ | | | | | |
| H8 | G-C | 0.064 | 0.068 | 6.61 × 10⁻⁰¹ | | | | | |
| H8 | A-T | 0.057 | 0.076 | 6.52 × 10⁻⁰² | | | | | |
| H8 | G-T | 0.645 | 0.484 | 2.83 × 10⁻¹⁵ | | | | | |
| H9 | | | | 1.88 × 10⁻¹⁵ | | | X | | X |
| H9 | A-C | 0.245 | 0.395 | 3.71 × 10⁻¹⁵ | | | | | |
| H9 | G-C | 0.114 | 0.117 | 8.31 × 10⁻⁰¹ | | | | | |
| H9 | A-A | 0.047 | 0.053 | 5.14 × 10⁻⁰¹ | | | | | |
| H9 | G-A | 0.594 | 0.435 | 9.04 × 10⁻¹⁵ | | | | | |
| H10 | | | | 2.16 × 10⁻¹³ | | | | X | X |
| H10 | C-C | 0.296 | 0.440 | 3.28 × 10⁻¹³ | | | | | |
| H10 | T-C | 0.064 | 0.072 | 4.49 × 10⁻⁰¹ | | | | | |
| H10 | T-A | 0.640 | 0.488 | 8.39 × 10⁻¹⁴ | | | | | |
| H11 | | | | 1.13 × 10⁻¹³ | X | X | X | | |
| H11 | C-C-A | 0.232 | 0.363 | 3.51 × 10⁻¹² | | | | | |
| H11 | T-C-A | 0.060 | 0.087 | 1.15 × 10⁻⁰² | | | | | |
| H11 | C-T-G | 0.036 | 0.027 | 1.84 × 10⁻⁰¹ | | | | | |
| H11 | T-T-G | 0.672 | 0.524 | 1.98 × 10⁻¹³ | | | | | |
| H12 | | | | 1.35 × 10⁻¹³ | X | X | | X | |
| H12 | C-C-C | 0.180 | 0.292 | 1.30 × 10⁻¹⁰ | | | | | |
| H12 | T-C-C | 0.057 | 0.084 | 1.05 × 10⁻⁰² | | | | | |
| H12 | T-T-C | 0.063 | 0.066 | 7.27 × 10⁻⁰¹ | | | | | |
| H12 | C-C-T | 0.054 | 0.074 | 4.06 × 10⁻⁰² | | | | | |
| H12 | C-T-T | 0.036 | 0.026 | 1.75 × 10⁻⁰¹ | | | | | |
| H12 | T-T-T | 0.611 | 0.458 | 7.99 × 10⁻¹⁴ | | | | | |
| H13 | | | | 1.97 × 10⁻¹⁴ | X | X | | | X |
| H13 | C-C-C | 0.190 | 0.316 | 1.74 × 10⁻¹² | | | | | |
| H13 | T-C-C | 0.057 | 0.084 | 1.11 × 10⁻⁰² | | | | | |
| H13 | T-T-C | 0.113 | 0.114 | 9.55 × 10⁻⁰¹ | | | | | |
| H13 | C-C-A | 0.044 | 0.051 | 3.96 × 10⁻⁰¹ | | | | | |
| H13 | C-T-A | 0.036 | 0.024 | 1.13 × 10⁻⁰¹ | | | | | |
| H13 | T-T-A | 0.561 | 0.411 | 3.58 × 10⁻¹³ | | | | | |
| H14 | | | | 1.10 × 10⁻¹⁴ | | X | X | X | |
| H14 | C-A-C | 0.235 | 0.373 | 2.38 × 10⁻¹³ | | | | | |
| H14 | T-G-C | 0.063 | 0.067 | 7.08 × 10⁻⁰¹ | | | | | |
| H14 | C-A-T | 0.057 | 0.076 | 6.01 × 10⁻⁰² | | | | | |
| H14 | T-G-T | 0.645 | 0.484 | 2.35 × 10⁻¹⁵ | | | | | |
| H15 | | | | 1.33 × 10⁻¹⁵ | | X | X | | X |
| H15 | C-A-C | 0.245 | 0.397 | 2.67 × 10⁻¹⁵ | | | | | |
| H15 | T-G-C | 0.113 | 0.116 | 8.53 × 10⁻⁰¹ | | | | | |
| H15 | C-A-A | 0.047 | 0.053 | 4.95 × 10⁻⁰¹ | | | | | |
| H15 | T-G-A | 0.595 | 0.435 | 6.76 × 10⁻¹⁵ | | | | | |
| H16 | | | | 3.20 × 10⁻¹⁴ | | | X | X | X |
| H16 | A-C-C | 0.234 | 0.372 | 2.52 × 10⁻¹³ | | | | | |
| H16 | G-C-C | 0.062 | 0.069 | 5.38 × 10⁻⁰¹ | | | | | |
| H16 | A-T-C | 0.012 | 0.023 | 3.56 × 10⁻⁰² | | | | | |
| H16 | G-T-C | 0.052 | 0.048 | 6.96 × 10⁻⁰¹ | | | | | |
| H16 | A-T-A | 0.045 | 0.053 | 3.65 × 10⁻⁰¹ | | | | | |
| H16 | G-T-A | 0.595 | 0.435 | 6.42 × 10⁻¹⁵ | | | | | |
| H17 | | | | 2.12 × 10⁻¹³ | X | X | X | X | |
| H17 | C-C-A-C | 0.179 | 0.291 | 1.43 × 10⁻¹⁰ | | | | | |
| H17 | T-C-A-C | 0.057 | 0.084 | 9.97 × 10⁻⁰³ | | | | | |
| H17 | T-T-G-C | 0.063 | 0.067 | 7.12 × 10⁻⁰¹ | | | | | |
| H17 | C-C-A-T | 0.054 | 0.073 | 5.40 × 10⁻⁰² | | | | | |
| H17 | C-T-G-T | 0.036 | 0.026 | 1.78 × 10⁻⁰¹ | | | | | |
| H17 | T-T-G-T | 0.611 | 0.459 | 1.25 × 10⁻¹³ | | | | | |
| H18 | | | | 2.62 × 10⁻¹⁴ | X | X | X | | X |
| H18 | C-C-A-C | 0.189 | 0.315 | 1.80 × 10⁻¹² | | | | | |
| H18 | T-C-A-C | 0.057 | 0.084 | 1.06 × 10⁻⁰² | | | | | |
| H18 | T-T-G-C | 0.113 | 0.114 | 9.26 × 10⁻⁰¹ | | | | | |
| H18 | C-C-A-A | 0.044 | 0.050 | 4.93 × 10⁻⁰¹ | | | | | |
| H18 | C-T-G-A | 0.036 | 0.024 | 1.15 × 10⁻⁰¹ | | | | | |
| H18 | T-T-G-A | 0.562 | 0.413 | 5.09 × 10⁻¹³ | | | | | |
| H19 | | | | 2.42 × 10⁻¹⁴ | | X | X | X | X |
| H19 | C-A-C-C | 0.234 | 0.373 | 1.90 × 10⁻¹³ | | | | | |
| H19 | T-G-C-C | 0.062 | 0.067 | 5.76 × 10⁻⁰¹ | | | | | |
| H19 | C-A-T-C | 0.012 | 0.023 | 3.51 × 10⁻⁰² | | | | | |
| H19 | T-G-T-C | 0.052 | 0.049 | 7.09 × 10⁻⁰¹ | | | | | |
| H19 | C-A-T-A | 0.045 | 0.054 | 3.48 × 10⁻⁰¹ | | | | | |
| H19 | T-G-T-A | 0.596 | 0.434 | 4.97 × 10⁻¹⁵ | | | | | |
| H20 | | | | 2.24 × 10⁻¹³ | X | X | X | X | X |
| H20 | C-C-A-C-C | 0.178 | 0.291 | 9.96 × 10⁻¹¹ | | | | | |
| H20 | T-C-A-C-C | 0.057 | 0.084 | 9.51 × 10⁻⁰³ | | | | | |
| H20 | T-T-G-C-C | 0.062 | 0.067 | 5.78 × 10⁻⁰¹ | | | | | |
| H20 | C-C-A-T-C | 0.011 | 0.023 | 2.47 × 10⁻⁰² | | | | | |
| H20 | T-T-G-T-C | 0.052 | 0.047 | 5.97 × 10⁻⁰¹ | | | | | |
| H20 | C-C-A-T-A | 0.043 | 0.051 | 3.58 × 10⁻⁰¹ | | | | | |
| H20 | C-T-G-T-A | 0.036 | 0.024 | 1.05 × 10⁻⁰¹ | | | | | |
| H20 | T-T-G-T-A | 0.562 | 0.413 | 4.10 × 10⁻¹³ | | | | | |

(^{a}Order of alleles corresponds to the genomic order as reflected in the columns on the right.)

A subset of the affected sample were members of 352 nuclear families, and the results of a transmission disequilibrium test (TDT) were in accordance with the case-control findings which had shown association for the five chromosome 20 SNPs and the highest significance for rs2180439 (TDT P=7.22×10⁻⁷).

TABLE 6
Transmission disequilibrium test in 352 German nuclear families

| SNP | Chr. | Position [bp]^{a} | Transmitted | Untransmitted | P^{b} | OR (95% CI) |
|---|---|---|---|---|---|---|
| rs6137444 | 20 | 21,733,639 | 182 (C) | 255 (C) | 4.79 × 10⁻⁴ | 1.40 (1.16-1.69) |
| rs2180439 | 20 | 21,801,100 | 172 (C) | 277 (C) | 7.22 × 10⁻⁷ | 1.61 (1.33-1.95) |
| rs1998076 | 20 | 21,828,045 | 170 (A) | 270 (A) | 1.87 × 10⁻⁶ | 1.59 (1.31-1.92) |
| rs201571 | 20 | 21,961,514 | 168 (C) | 258 (C) | 1.30 × 10⁻⁵ | 1.54 (1.26-1.86) |
| rs6113491 | 20 | 22,005,415 | 193 (C) | 288 (C) | 1.48 × 10⁻⁵ | 1.49 (1.24-1.79) |

(^{a}Build 36; ^{b}Association P value)

We also performed an initial step to investigate the importance of this locus in non-German populations by analyzing a modestly sized Australian sample for the five chromosome 20 SNPs. Again, rs2180439 showed significant association.

TABLE 7
Replication analysis in Australian AGA sample

| | | 141 cases^{a} vs. 150 controls | | | | 60 cases^{b} vs. 150 controls | | | |
|---|---|---|---|---|---|---|---|---|---|
| | Minor | MAF | MAF | | | MAF | MAF | | |
| SNP | allele | cases | controls | P | OR (95% CI) | cases | controls | P | OR (95% CI) |
| rs6137444 | C | 0.298 | 0.343 | 0.247 | 1.23 (0.86-1.74) | 0.275 | 0.343 | 0.171 | 1.37 (0.86-2.19) |
| rs2180439 | C | 0.340 | 0.420 | 0.041 | 1.4 (1-1.96) | 0.317 | 0.420 | 0.045 | 1.56 (1-2.44) |
| rs1998076 | A | 0.344 | 0.417 | 0.064 | 1.36 (0.97-1.91) | 0.317 | 0.417 | 0.053 | 1.54 (0.99-2.41) |
| rs201571 | C | 0.309 | 0.417 | 0.0069 | 1.6 (1.14-2.25) | 0.292 | 0.417 | 0.018 | 1.73 (1.1-2.73) |
| rs6113491 | C | 0.401 | 0.477 | 0.066 | 1.36 (0.98-1.89) | 0.392 | 0.477 | 0.111 | 1.41 (0.92-2.17) |

(^{a}AGA grade ≧ IIa; ^{b}AGA grade ≧ IV)

### EXAMPLE 4

### Phenotype

As regards phenotype, German GWAS cases (extreme) and controls (containing affecteds) differed from the replication cases (less extreme) and controls (unaffecteds). The genotype based ORs of rs2180439 in the four possible case-control comparisons showed an increase from the least extreme (replication cases/GWAS controls, OR=3.72, 95% CI 2.18-6.34) to the most extreme (GWAS cases/replication controls, OR=6.16, 95% CI 3.48-10.92) for the homozygotes (FIG. 2). Correlation of effect size and phenotypic difference supports an association between rs2180439 and AGA. However, this relation was less clear for the heterozygotes. We used logistic regression (Cordell H J, Clayton D G (2002), Am J Hum Genet 70:124-141) and tested for deviation from a multiplicative model on the OR scale in the combined data set. The test showed a tendency toward a dominant model for rs2180439 (P=0.023)

TABLE 8
Test for deviation from a multiplicative
model with two degrees of freedom
versus one degree of freedom in the
combined German data set

| SNP | P |
|---|---|
| rs6137444 | 0.0608 |
| rs2180439 | 0.0225 |
| rs1998076 | 0.0203 |
| rs201571 | 0.0583 |
| rs6113491 | 0.0025 |

We also found evidence that rs2180439 and rs6113491 contribute independently to the risk of AGA, and that rs6113491 showed a dominant effect.

TABLE 9
Tests for additional effects of further SNPs at the
chromosome 20 locus in the combined German data set

| | LD measure | P | |
|---|---|---|---|
| | r² with | Test for | Test for |
| | rs2180439 | independence^{a} | dominance^{b} |
| rs6137444 | 0.6 | 0.865 | n.d. |
| rs1998076 | 0.99 | 0.2634 | n.d. |
| rs201571 | 0.5 | 0.0367 | 0.3677 |
| rs6113491 | 0.44 | 0.0033 | 0.0192 |

(^{a}3 degrees of freedom versus 2 degrees of freedom;
^{b}4 degrees of freedom versus 3 degrees of freedom)

To observe this effect, however, use of the combined sample was necessary since rs6113491 did not remain significant after stratifying the GWAS data for rs2180439 (FIG. 1).

### EXAMPLE 5

### Interaction

We tested chromosome 20p11 for interaction with the primary associated SNP at the AR locus (rs1041668). Introduction of rs1041668 improved the model-fit significantly. However, neither the interaction terms with rs2180439 (P=0.975) nor with rs6113491 (P=0.984) yielded significant improvement of the model-fit. Thus, our data provide no evidence for interaction effects between the two regions.

### EXAMPLE 6

### Gene Contributing to the Development of AGA

Two expressed sequence tags, BQ013595 and BE789145, map to the associated region on 20p11 (FIG. 1). Since a gene contributing to the development of AGA would be expected to be expressed in the human scalp, we quantified the expression of BQ013595 and BE789145 in various human tissues including skin, hair and scalp. In addition, we included the closest reference sequence gene, PAX1 (paired box 1). BE789145 was not detectable in skin, hair or scalp and BQ013595 showed very low expression in hair and scalp. PAX1 was found to be expressed at very low levels in hair and skin, but showed considerable expression in the scalp (FIG. 3). Although PAX1 is located outside of the associated LD block, the expression data might suggest that PAX1 confers the AGA relevant effect at this locus and that a regulatory variant within the associated LD block may modulate its expression.

### EXAMPLE 7

### Etiological Fraction

The AGA risk allele T of rs2180439 has a frequency of 0.57 in our population based control sample of 383 individuals (before exclusion of the AGA affected controls for the GWAS step). The etiological fraction for rs2180439 is estimated at 0.32, underlining the importance of this locus in the development of AGA. The frequency of the risk allele varies world-wide from 0.03 to 0.86 (FIG. 4). This may account in part for population specific differences in AGA prevalence, but it does not explain the lower prevalence reported in South-East Asian populations (Paik, J. H., et al. (2001) N.I. Br J Dermatol 145, 95-99; Norwood O T (1975), South Med J 68, 1359-1365). It is of note that the chromosome 20 locus did not show linkage to AGA in a recent study (Hillmer, A. M. et al. (2008), Am J Hum Genet 82, 737-743). This supports the value of the association approach in detecting susceptibility genes for complex diseases (Risch, N., Merikangas, K. (1996), Science 273, 1516-1517).

1. A method for testing for a predisposition or presence of androgenetic alopecia comprising testing a sample obtained from a prospective patient or from a person suspected of carrying a predisposition for androgenetic alopecia, the method comprising determining
(a) the presence and/or quantity of a nucleic acid molecule selected from the group consisting of:
(i) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292;
(ii) a nucleic acid molecule the complementary strand of which hybridises under stringent conditions to the nucleic acid molecule of (a)(i), wherein said nucleic acid molecule has at a position corresponding to position 201 of any one of SEQ ID NOs:1 to 77 an adenosine, corresponding to position 201 of any one of SEQ ID NOs:78 to 149 a guanosine, corresponding to position 201 of any one of SEQ ID NOs:150 to 219 a thymidine or corresponding to position 201 of any one of SEQ ID NOs:220 to 292 a cytidine;
(iii) a nucleic acid molecule identical to any one of the nucleic acid molecules of (a)(i) or (a)(ii) wherein each thymidine is replaced by uridine; and/or
(b) the presence and/or quantity of a nucleic acid molecule selected from the group consisting of:
(i) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292;
(ii) a nucleic acid molecule the complementary strand of which hybridises under stringent conditions to the nucleic acid molecule of (b)(i), wherein said nucleic acid molecule has at a position corresponding to position 201 of any one of SEQ ID NOs: SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264 an adenosine, corresponding to position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215 a cytidine, corresponding to position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 a guanosine or corresponding to position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292 a thymidine;
(iii) a nucleic acid molecule identical to any one of the nucleic acid molecules of (b)(i) or (b)(ii) wherein each thymidine is replaced by uridine; and/or
(c) the presence and/or quantity of a nucleic acid molecule which is complementary to the nucleic acid molecule of (a) or (b); or
(d) the presence of a risk allele within the nucleic acid molecule of (a) as compared to the non-risk allele, wherein said risk allele is characterised in that it is an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292; or
(e) the presence of a risk allele within the nucleic acid molecule of (c) as compared to the non-risk allele, wherein said risk allele is characterised in that it is an thymidine at position 201 of any one of SEQ ID NOs:1 to 77, a cytidine at position 201 of any one of SEQ ID NOs:78 to 149, a adenosine at position 201 of any one of SEQ ID NOs:150 to 219 or a guanosine at position 201 of any one of SEQ ID NOs:220 to 292; or
(f) the presence of a non-risk allele within the nucleic acid molecule of (b) as compared to the risk allele, wherein said non-risk allele is characterised in that it is an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; or
(g) the presence of a non-risk allele within the nucleic acid molecule of (c) as compared to the risk allele, wherein said non-risk allele is characterised in that it is an thymidine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a guanosine at position 201 of any one of SEQ ID NOs: 3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a cytidine at position 201 of any one of SEQ ID NOs: 1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a adenosine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292; or
(h) the presence of a risk allele of a genetic marker of androgenetic alopecia characterised in that the genetic marker is located in a chromosomal region of human chromosome 20p11 and wherein the genetic marker is selected from the group consisting of a single nucleotide polymorphism (SNP), variable number of tandem repeat (VNTR), microsatellites or short tandem repeats (STR)
and wherein the presence of said genetic marker, or said risk allele is indicative of androgenetic alopecia or a predisposition for androgenetic alopecia and wherein the presence of said non-risk allele is indicative of the absence of androgenetic alopecia or a predisposition for androgenetic alopecia.

2. The method of claim 1, wherein said determining of the presence of said genetic marker, said nucleic acid molecule or the presence of said risk or non-risk allele comprises PCR based techniques, RFLP- or AFLP-based techniques, DNA sequencing-based techniques, hybridization-based techniques, single-strand conformation polymorphism analysis (SSCA), denaturating gradient gel electrophoresis (DGGE), mismatch cleavage detection, heteroduplex analysis, primer extension-based techniques, and 5′-nuclease assay-based techniques.

3. The method of claim 1, further comprising detecting an alteration in the expression of a nucleic acid molecule encoding a polypeptide having PAX1 function, wherein said nucleic acid molecule comprises:
(a) a nucleic acid molecule encoding a polypeptide having the amino acid sequence of SEQ ID NO:293;
(b) a nucleic acid molecule having the DNA sequence of SEQ ID NO:294;
(c) a nucleic acid molecule having the sequence of SEQ ID NO:294, wherein each thymidine is replaced by uridine;
wherein the alteration of expression as compared to the amount of nucleic acid molecule expression in the control sample is indicative of androgenetic alopecia or a predisposition for androgenetic alopecia.

4. The method according to claim 1, wherein the sample is blood, serum, plasma, fetal tissue, saliva, urine, mucosal tissue, mucus, vaginal tissue, fetal tissue obtained from the vagina, skin, hair, hair follicle or another human tissue.

5. A genetic marker for use in the diagnosis of androgenetic alopecia characterised in that the genetic marker is located in a chromosomal region of human chromosome 20p11 and wherein the genetic marker is selected from the group consisting of a single nucleotide polymorphism (SNP), variable number of tandem repeat (VNTR), microsatellites or short tandem repeats (STR).

6. The method of claim 1 or the genetic marker of claim 5, wherein the chromosomal region of human chromosome 20p11 consists of the region having the sequence of SEQ ID NO:1 at the 5′ end and the sequence of SEQ ID NO:292 at the 3′ end.

7. The method of claim 1 or the genetic marker of claim 5, wherein the chromosomal region of human chromosome 20p11 comprises at least one of SEQ ID NOs:1 to 292.

8. The method of claim 1 or the genetic marker of claim 5, wherein the genetic marker is a SNP selected from the group consisting of the SNPs comprised in any one of SEQ ID NOs:1 to 292.

9. A nucleic acid molecule selected from the group consisting of:
(a) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:1 to 77, a guanosine at position 201 of any one of SEQ ID NOs:78 to 149, a thymidine at position 201 of any one of SEQ ID NOs:150 to 219 or a cytidine at position 201 of any one of SEQ ID NOs:220 to 292;
(b) a nucleic acid molecule the complementary strand of which hybridises under stringent conditions to the nucleic acid molecule of (a), wherein said nucleic acid molecule has at a position corresponding to position 201 of any one of SEQ ID NOs:1 to 77 an adenosine, corresponding to position 201 of any one of SEQ ID NOs:78 to 149 a guanosine, corresponding to position 201 of any one of SEQ ID NOs:150 to 219 a thymidine or corresponding to position 201 of any one of SEQ ID NOs:220 to 292 a cytidine;
(c) a nucleic acid molecule identical to any one of the nucleic acid molecules of (a) or (b) wherein each thymidine is replaced by uridine.

10. A nucleic acid molecule selected from the group consisting of:
(a) a nucleic acid molecule consisting of at least 17 consecutive nucleotides of any of SEQ ID NOs:1 to 292; wherein the at least 17 consecutive nucleotides contain an adenosine at position 201 of any one of SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264, a cytidine at position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215, a guanosine at position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 or a thymidine at position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292;
(b) a nucleic acid molecule the complementary strand of which hybridises under stringent conditions to the nucleic acid molecule of (a), wherein said nucleic acid molecule has at a position corresponding to position 201 of any one of SEQ ID NOs: SEQ ID NOs:78, 79, 83, 86, 87, 90, 91, 93, 94, 96, 98 to 109, 111, 112, 116 to 123, 126, 128 to 142, 144 to 149, 151, 155, 158 to 160, 162, 165, 176, 177, 182, 184, 204, 207, 212, 217, 221, 226, 228, 230, 232, 233, 235, 239, 242, 243, 251, 253, 254, 259, 261 or 264 an adenosine, corresponding to position 201 of any one of SEQ ID NOs:3, 7, 25, 34, 35, 38, 39, 48, 50, 51, 54, 57, 67, 76, 80, 89, 95, 110, 114, 115, 125, 150, 152, 154, 156, 157, 164, 166, 168 to 175, 179 to 181, 183, 185, 188 to 191, 193, 195 to 203, 205, 206, 208 to 211, 213 or 215 a cytidine, corresponding to position 201 of any one of SEQ ID NOs:1, 2, 4, 5, 8 to 11, 13, 14, 16, 18, 20 to 23, 26 to 28, 30 to 33, 36, 37, 41 to 47, 49, 52, 53, 55, 56, 60 to 66, 68 to 75, 77, 153, 161, 163, 167, 178, 186, 187, 192, 194, 214, 216, 218 to 220, 222 to 224, 236, 241, 245, 252, 266, 273 to 275, 277, 279 or 284 a guanosine or corresponding to position 201 of any one of SEQ ID NOs:6, 12, 15, 17, 19, 24, 29, 40, 58, 59, 81, 82, 84, 85, 88, 92, 97, 113, 142, 127, 143, 225, 227, 229, 231, 234, 237, 238, 240, 244, 246 to 250, 255, 256 to 258, 260, 262, 263, 265, 267 to 272, 276, 278, 280 to 283, 285 to 292 a thymidine;
(c) a nucleic acid molecule identical to any one of the nucleic acid molecules of (a) or (b) wherein each thymidine is replaced by uridine.

11. The method of claim 1 or the nucleic acid molecule of claim 9, wherein the nucleic acid molecule is genomic DNA.

12. A nucleic acid molecule which is complementary to the nucleic acid molecule of claim 9 or 10.

13. A vector comprising the nucleic acid molecule of any one of claim 9 or 10.

14. A host cell genetically engineered with the nucleic acid molecule of claim 9 or 10.

15. A diagnostic composition for detection of androgenetic alopecia or a predisposition for androgenetic alopecia comprising the nucleic acid molecule of claim 9 or 10.

16. A Kit comprising the nucleic acid molecule of claim 9 or 10.
